(19) United States
(12) Patent Application Publication
(10) Pub. No.: US 2019/0382655 A1
(43) Pub. Date: Dec. 19, 2019

(54) LUMINESCENT NANOCRYSTAL COMPLEX

(71) Applicant: DIC Corporation, Tokyo (JP)

(72) Inventors: Yasuhiro Kuwana, Kitaadachi-gun (JP); Koichi Endou, Kitaadachi-gun (JP); Kazuaki Hatsusaka, Kitaadachi-gun (JP); Yoshio Aoki, Kitaadachi-gun (JP); Hidehiko Yamaguchi, Kitaadachi-gun (JP)

(73) Assignee: DIC Corporation, Tokyo (JP)

(21) Appl. No.: 16/464,442

(22) PCT Filed: Dec. 7, 2017
(86) PCT No.: PCT/JP2017/043958

§ 371 Date: May 28, 2019

(30) Foreign Application Priority Data
Dec. 12, 2016 (JP) 2016-240338

(51) Int. Cl. C09K 11/70; C07C 321/10; C07C 323/07; C07C 33/46; C07C 57/58; C07C 63/66; C07D 213/30; C07D 333/16; C07F 9/53; C08F 2/44; C09K 11/56; C07C 211/29

(52) U.S. Cl. CPC C09K 11/70; C07C 321/10; C07C 323/07; C07C 33/46; C07C 57/58; C07C 63/66; B82Y 40/00; C07D 333/16; C07F 9/53; C08F 2/44; C09K 11/56; C07C 211/29; C07D 213/30

(57) ABSTRACT

“Object” A problem to be solved by the present invention is to provide a luminescent nanocrystal complex that tends to disperse orderly in a polymer matrix and is superior in dispersibility in structurally mesogenic crosslinkable polymer matrices. “Solution” The present invention is a luminescent nanocrystal complex that contains luminescent nanocrystals and a surface-modifying compound that modifies the surface of the luminescent nanocrystals. The surface-modifying compound has a mesogenic backbone and a group that binds to the surface of the luminescent nanocrystals.

### TECHNICAL FIELD

The present invention relates to a luminescent nanocrystal complex.

### BACKGROUND ART

Luminescent nanocrystals, which are small particles each formed by hundreds to tens of thousands of atoms and approximately nanometers to hundred nanometers in size and examples of which include quantum dots and quantum rods, emit bright fluorescence with a small half width and different wavelengths depending on the particle diameter by virtue of their quantum size effect and many-electron effect. Quantum dots and other luminescent nanocrystals, moreover, enables prolonged observation of fluorescence owing to their brightness higher than achieved with organic fluorochromes or fluorescent proteins and resistance to fading caused by excitation light. For these reasons, quantum dots and other luminescent nanocrystals have been in focus as new materials in various technical fields, such as fluorescent probes for biological labeling, lighting, displays, and batteries.

Surface atoms of quantum dots, quantum rods, or other luminescent nanocrystals are generally known to be highly reactive and often cause aggregation between the particles because of their potential to become coordination sites. In general, therefore, quantum dots are passivated through the protection of their surface atoms with an organic group (capping). Such an organic group that protects surface atoms of quantum dots is referred to as, for example, a capping agent or ligand and has been the subject of a wide variety of research and development.

For example, NPL 1 discloses a ligand that has a polyethylene glycol backbone with a thiol group at one end and a sugar capable of binding to a particular protein, an example of the sugar being N-acetylgalactosamine, at the other end. The authors synthesized quantum dots by modifying the surface of CdTe particles with this ligand via the thiol group.

NPL 2 discloses that the authors prepared nanoparticles by modifying glutathione-coated CdSeTe/CdS quantum dots with anti-HER2 antibodies via carboxyl or amino groups derived from the glutathione residue and injected the nanoparticles into model mice transplanted with human breast cancer KPL-4 cells with overexpressed HER2 receptors for light-field and near-infrared fluorescence imaging. This means, the standard approach to using quantum dots as a fluorescent probe for biological labeling is to modify the surface of the quantum dots with an antibody or a ligand for a receptor.

In PTL 1, it is described that Cd/ZnSeS core-shell quantum dots were coordinated with an organic ligand tri-n-octylphosphine (TOP), and then the TOP was replaced with a pyridine ligand.

In PTL 2, hexadecylamine (HDA)-capped CdSe nanoparticles were actually synthesized. Besides capping agents that are common Lewis base compounds, which are capable of donor-type coordination to the surface of quantum dots, the inventors mention mercapto-functionalized amine or mercaptocarboxylic acid, styrene-functionalized amine, and phosphine or phosphine oxide ligands.

PTL 3 discloses a ligand that is a benzene ring substituted with a carboxylic acid group at one end and three alkyl ether chains at the other end, with the alkyl ether chains having a vinyl group bound thereto. The authors synthesized InP/ZnS core-shell nanoparticles whose surface was modified with this ligand via the carboxylic group. PTL 3 also discloses crosslinking the vinyl groups together using a Hoveyda-Grubbs catalyst and that the nanoparticles can be incorporated into silicone-based materials.

In PTL 4, moreover, it is described that perhydropolysilazane-coated quantum-dot nanoparticles were synthesized to be compared with InP/ZnS quantum dots coated with a terminal amino-containing resin or substituted with an amino-containing thiol ligand. According to PTL 4, the inventor demonstrates therein that the perhydropolysilazane-coated quantum-dot nanoparticles achieve higher luminescence intensity.

### CITATION LIST

### Patent Literature

PTL 1: Japanese Unexamined Patent Application Publication (Translation of PCT Application) No. 2010-532409
PTL 2: Japanese Unexamined Patent Application Publication (Translation of PCT Application) No. 2007-537886
PTL 3: Japanese Unexamined Patent Application Publication (Translation of PCT Application) No. 2012-507588
PTL 4: Japanese Unexamined Patent Application Publication (Translation of PCT Application) No. 2015-127362

### Non Patent Literature

NPL 1: K. Niikura, et al. ChemBioChem, vol. 8, 379 (2007)
NPL 2: Int. J. Mol. Sci. 2008, 9 (10), 2044-2061

### SUMMARY OF INVENTION

### Technical Problem

Quantum dots or other luminescent nanocrystals surface-modified with a ligand are usually not used alone. They are usually mixed with other substances, such as solvent and a polymeric resin, before use. In all of PTL 1 to 4 above, however, quantum dots or other luminescent nanocrystals are capped with ligands in order to prevent the aggregation of the particles themselves or to protect the particles from their surrounding chemical or electrical environment. The ligands described in PTL 1 to 4 above, moreover, are disadvantageous in that quantum dots or other luminescent nanocrystals modified therewith, modified with a compound having a structure with a non-mesogenic backbone, tend to go into disorder when dispersed in a polymer matrix, and that quantum dots or other luminescent nanocrystals modified therewith are not very dispersible in polymer matrices, in particular structurally mesogenic crosslinkable polymer matrices, because of low compatibility between the polymer matrix and the quantum dots or other luminescent nanocrystals.

As for anisotropic luminescent nanocrystals, such as quantum rods, the quantum rods need to be arranged in a particular direction to emit polarized light. With the ligands described in PTL 1 to 4 above, however, efficient extraction of light from quantum rods has yet to be achieved because of difficulty aligning the quantum rods orderly.

On the other hand, the ligands described in NPL 1 and 2, for modifying the surface of quantum dots, have the function not only of protecting the particles from aggregation therebetween and from their surrounding chemical environment, but also of having a moiety that incorporates a specific bond with a protein. However, these ligands are disadvantageous in that quantum dots, for example, surface-modified with a component of biological origin like a protein are very difficult to handle because such biological components can fulfill their function under the very limited conditions (solvent, pH, temperature, and ionic strength) of in vivo environments.

A problem to be solved by the present invention is therefore to provide a luminescent nanocrystal complex that tends to disperse orderly in a polymer matrix and is superior in dispersibility in structurally mesogenic crosslinkable polymer matrices by virtue of modification of the surface of luminescent nanocrystals, such as quantum dots or quantum rods, with a structurally mesogenic compound.

Another problem to be solved by the present invention is to provide a luminescent nanocrystal composition that makes it easy to handle surface-modified luminescent nanocrystals and at the same time tends to disperse orderly over a broad temperature range.

### Solution to Problem

After extensive research to solve the above problems, the inventors found that the problems can be solved by applying a liquid crystal layer that contains a particular liquid crystal compound to a liquid crystal display device that uses quantum dots or other luminescent nanocrystals as a color filter. Based on these findings, the inventors completed the present invention. That is, the present invention relates to a luminescent nanocrystal complex. The complex includes luminescent nanocrystals and a surface-modifying compound that modifies the surface of the luminescent nanocrystals, and the surface-modifying compound has a mesogenic group and a group that binds to the surface of the luminescent nanocrystals.

### Advantageous Effects of Invention

The luminescent nanocrystal complex according to the present invention offers improved efficiency in light emission by virtue of uniform dispersion of luminescent nanocrystals achieved through the modification of the surface of the luminescent nanocrystals with a molecule having a mesogenic group. The complex also offers improved durability of the phosphor.

The luminescent nanocrystal complex according to the present invention improves in alignment and achieves a higher degree of polarization if the luminescent nanocrystals are a quantum-rod phosphor.

The luminescent nanocrystal complex according to the present invention offers reduced concentration quenching by virtue of controlled aggregation that owes to a uniform apparent shape and a large volume of the luminescent nanocrystal complex provided by surface modification with a compound having a rigid mesogenic group.

### DESCRIPTION OF EMBODIMENTS

As stated above, the present invention is a luminescent nanocrystal complex that includes luminescent nanocrystals and a surface-modifying compound that modifies the surface of the luminescent nanocrystals, and the surface-modifying compound has a mesogenic backbone and a group that binds to the surface of the luminescent nanocrystals.

In the present invention, uniform dispersion of the luminescent nanocrystal complex improves its efficiency in light emission. The durability of the phosphor is also improved. By virtue of having a uniform apparent shape and a large volume and containing a ligand having a structurally rigid mesogenic backbone as an essential ingredient, the luminescent nanocrystal complex according to the present invention is not very variable in excluded volume. The particles of the luminescent nanocrystal complex can therefore be present with an adequate distance therebetween. As a result, the inventors believe, the nanocrystals are unlikely to aggregate, and concentration quenching does not occur easily.

The luminescent nanocrystals according to the present invention improve in alignment and achieve a higher degree of polarization if applied to a quantum-rod phosphor.

A luminescent nanocrystal complex according to the present invention has luminescent nanocrystals and a surface-modifying compound (ligand) that modifies the surface of the luminescent nanocrystals. The term “nanocrystal” herein refers to a particle, preferably having at least one dimension of 100 nm or less. The shape of the nanocrystals may be any geometric shape and may be symmetric or asymmetric. Specific examples of shapes of the nanocrystals include elongated, rod-like, round (spherical), ellipsoidal, pyramidal, disk-shaped, branched, mesh-like, and any irregular shapes. In certain embodiments, the nanocrystals are preferably quantum dots or quantum rods.

The luminescent nanocrystals preferably have a core that contains at least a first semiconductor material and a shell that covers the core and contains a second semiconductor material that is the same as or different than in the core.

The luminescent nanocrystals may therefore be composed of a core containing at least a first semiconductor material and a shell containing a second semiconductor material, with the first and second semiconductor materials being the same or different. The core and/or shell may both contain an extra, third semiconductor material besides the first and/or second semiconductor materials. The covering the core as used here only requires that at least part of the core be covered.

Moreover, the luminescent nanocrystals preferably have a core that contains at least one first semiconductor material, a first shell that covers the core and contains a second semiconductor material that is the same as or different than in the core, and optionally a second shell that contains a third semiconductor material that is the same as or different than in the first shell.

The luminescent nanocrystals according to the present invention therefore, preferably, have at least one of the three structures of the form in which the nanocrystals have a core that contains a first semiconductor material and a shell that covers the core and contains a second semiconductor material that is the same as in the core, or the mode in which the nanocrystals are made of one or two or more semiconductor materials (=the structure in which a core is the only component (also referred to as the core structure)); the form in which the nanocrystals have a core that contains a first semiconductor material and a shell that contains a second semiconductor material different than in the core or a similar form, or the core/shell structure; and the form in which the nanocrystals have a core that contains a first semiconductor material, a first shell that covers the core and contains a second semiconductor material different than in the core, and a second shell that contains a third semiconductor material different than in the first shell, or the core/shell/shell structure.

As stated above, the luminescent nanocrystals according to the present invention preferably include the three forms of the core structure, the core/shell structure, and the core/shell/shell structure. In this case, the core may be a mixed crystal that contains two or more semiconductor materials (e.g., CdSe+CdS, CIS+ZnS, InP+ZnS, or InP+ZnO). The shell, too, may be a mixed crystal that contains two or more semiconductor materials.

The luminescent nanocrystals according to the present invention may be in contact with, besides the surface-modifying compound according to the present invention, a molecule compatible with the luminescent nanocrystals.

The compatible molecule is a non-polymer or polymer that has a functional group compatible with the luminescent nanocrystals. The compatible functional group can be of any type but preferably is a group that contains one element selected from the group consisting of nitrogen, oxygen, sulfur, and phosphor. Examples include organic sulfur, organic phosphoric acid, pyrrolidone, pyridine, amino, amide, isocyanate, carbonyl, and hydroxy groups.

Semiconductor material(s) according to the present invention is preferably one or two or more selected from the group consisting of group II-VI semiconductors, group III-V semiconductors, group I-III-VI semiconductors, group IV semiconductors, and group I-II-IV-VI semiconductors. For the first, second, and third semiconductor materials according to the present invention, preferred examples are the same as listed for the semiconductor material(s).

Specifically, semiconductor material(s) according to the present invention is at least one or more selected from the group consisting of CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnSe, CdHgS, CdHgSe, CdHgSe, HgZnS, HgZnSe, CdHgZnTe, CdZnSeS, CdZnSeTe, CdZnSTe, CdHgSeS, CdHgSeTe, CdHgSTe, HgZnSeS, HgZnSeTe, and HgZnSTe; GaN, GaP, GaAs, GaSb, AlN, Alp, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and InAlPSb; SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe; Si, Ge, SiC, SiGe, AgInSe2, CuGaSe2, CuInS2, CuGaS2, CuInSe2, AgInS2, AgGaSe2, AgGaS2, C, Si, and Ge. One of these compound semiconductors may be used alone, or two or more may be mixed. It is more preferred that at least one or more be selected from the group consisting of CdS, CdSe, CdTe, ZnS, ZnSe, ZnTe, ZnO, HgS, HgSe, HgTe, InP, InAs, InSb, GaP, GaAs, GaSb, AgInS₂, AgInSe₂, AgInTe₂, AgGaS₂, AgGaSe₂, AgGaTe₂, CuInS₂, CuInSe₂, CuInTe₂, CuGaS₂, CuGaSe₂, CuGaTe₂, Si, C, Ge, and Cu₂ZnSnS₄. One of these compound semiconductors may be used alone, or two or more may be mixed.

The luminescent nanocrystals according to the present invention preferably include at least one type of nanocrystals selected from the group consisting of red light-emitting nanocrystals, which emit red light, green light-emitting nanocrystals, which emit green light, blue light-emitting nanocrystals, which emit blue light, and yellow light-emitting nanocrystals, which emit yellow light. In general, the color of the light emitted by luminescent nanocrystals depends on the particle diameter, according to the solution of the Schrodinger wave equation of a potential well model, but also depends on the energy gap the luminescent nanocrystals have. The color of the emitted light is therefore selected by adjusting the luminescent nanocrystals used and their particle diameter.

In the present invention, the upper limit for the peak wavelength of the fluorescence spectrum of red light-emitting nanocrystals, which emit red light, is preferably 665 nm, 663 nm, 660 nm, 658 nm, 655 nm, 653 nm, 651 nm, 650 nm, 647 nm, 645 nm, 643 nm, 640 nm, 637 nm, 635 nm, 632 nm, or 630 nm. The lower limit for the same peak wavelength is preferably 628 nm, 625 nm, 623 nm, 620 nm, 615 nm, 610 nm, 607 nm, or 605 nm.

In the present invention, the upper limit for the peak wavelength of the fluorescence spectrum of green light-emitting nanocrystals, which emit green light, is preferably 560 nm, 557 nm, 555 nm, 550 nm, 547 nm, 545 nm, 543 nm, 540 nm, 537 nm, 535 nm, 532 nm, or 530 nm. The lower limit for the same peak wavelength is preferably 528 nm, 525 nm, 523 nm, 520 nm, 515 nm, 510 nm, 507 nm, 505 nm, 503 nm, or 500 nm.

In the present invention, the upper limit for the peak wavelength of the fluorescence spectrum of blue light-emitting nanocrystals, which emit blue light, is preferably 480 nm, 477 nm, 475 nm, 470 nm, 467 nm, 465 nm, 463 nm, 460 nm, 457 nm, 455 nm, 452 nm, or 450 nm. The lower limit for the same peak wavelength is preferably 450 nm, 445 nm, 440 nm, 435 nm, 430 nm, 428 nm, 425 nm, 422 nm, or 420 nm.

In the present invention, the semiconductor material(s) used in red light-emitting nanocrystals, which emit red light, desirably has a peak emission wavelength that falls within the range of 635 nm±30 nm. Likewise, the semiconductor material(s) used in green light-emitting nanocrystals, which emit green light, desirably has a peak emission wavelength that falls within the range of 530 nm±30 nm, and the semiconductor material(s) used in blue light-emitting nanocrystals, which emit blue light, desirably has a peak emission wavelength that falls within the range of 450 nm±30 nm.

The lower limit for the fluorescence quantum yield of the luminescent nanocrystals according to the present invention is preferably 40% or more, 30% or more, 20% or more, or 10% or more, in order of preference.

The upper limit for the half width of the fluorescence spectrum of the luminescent nanocrystals according to the present invention is preferably 60 nm or less, 55 nm or less, 50 nm or less, or 45 nm or less, in order of preference.

The upper limit for the particle diameter (primary particles) of red light-emitting nanocrystals according to the present invention is preferably 50 nm or less, 40 nm or less, 30 nm or less, or 20 nm or less, in order of preference.

The upper and lower limits for the peak wavelength of red light-emitting nanocrystals according to the present invention are 665 nm and 605 nm, respectively. The compound(s) and its particle diameter are selected to match this peak wavelength. Likewise, the upper and lower limits for the peak wavelength of green light-emitting nanocrystals are 560 nm and 500 nm, respectively, and those for the peak wavelength of blue light-emitting nanocrystals are 420 nm and 480 nm, respectively. For each type, the compound(s) and its particle diameter are selected to match this peak wavelength.

A liquid crystal display element according to the present invention includes at least one pixel. The colors constituting the pixel are given by three pixels close to one another, and each pixel contains a different type of nanocrystals that emit light in red (e.g., luminescent nanocrystals of CdSe, rod-shaped luminescent nanocrystals of CdSe, core-shell rod-shaped luminescent nanocrystals with a CdS shell portion and a CdSe inner, core portion, core-shell rod-shaped luminescent nanocrystals with a CdS shell portion and a ZnSe inner, core portion, core-shell luminescent nanocrystals with a CdS shell portion and a CdSe inner, core portion, core-shell luminescent nanocrystals with a CdS shell portion and a ZnSe inner, core portion, luminescent mixed nanocrystals of CdSe and ZnS, rod-shaped luminescent mixed nanocrystals of CdSe and ZnS, luminescent nanocrystals of InP, luminescent nanocrystals of InP, rod-shaped luminescent nanocrystals of InP, luminescent mixed nanocrystals of CdSe and CdS, rod-shaped luminescent mixed nanocrystals of CdSe and CdS, luminescent mixed nanocrystals of ZnSe and CdS, or rod-shaped luminescent mixed nanocrystals of ZnSe and CdS), green (e.g., luminescent nanocrystals of CdSe, rod-shaped luminescent nanocrystals of CdSe, luminescent mixed nanocrystals of CdSe and ZnS, or rod-shaped luminescent mixed nanocrystals of CdSe and ZnS), or blue (luminescent nanocrystals of ZnSe, rod-shaped luminescent nanocrystals of ZnSe, luminescent nanocrystals of ZnS, rod-shaped luminescent nanocrystals of ZnS, core-shell luminescent nanocrystals with a ZnSe shell portion and a ZnS inner, core portion, core-shell rod-shaped luminescent nanocrystals with a ZnSe shell portion and a ZnS—CdS inner, core portion, or rod-shaped luminescent nanocrystals of CdS). Other colors (e.g., yellow light-emitting nanocrystals may also be used.

If luminescent nanocrystals according to the present invention are so-called quantum rods, the length (average length) along the major axis of the quantum rods is preferably between 15 and 120 nm, preferably between 20 and 80 nm, more preferably between 25 and 70 nm.

If 20 nm or longer along their major axis, the inventors believe, the quantum rods have an anisotropy that effectively gives them the capability of emitting polarized light. If 120 nm or shorter along their major axis, the quantum rods do not impair the surface-modifying compound's capability of ordered dispersion.

The length (average length) along the minor axis of the quantum rods is preferably between 1 and 11 nm, more preferably between 2 and 8 nm, even more preferably between 3 and 7 nm.

As for the shape of quantum rods according to the present invention, they only need to be long objects that extend in a particular direction. Examples include cylindrical, prismatic, pyramidal, and conical shapes.

The aspect ratio of quantum rods according to the present invention (average length along the major axis of the quantum rods/average length along the minor axis of the quantum rods) is preferably between 3 and 30, more preferably between 4 and 20, even more preferably between 5 and 10.

The material(s) forming the quantum rods is not critical. The materials listed above for luminescent nanocrystals can be suitably used.

The average particle diameter (primary particles) of luminescent nanocrystals herein can be measured by TEM observation. In general, the average particle diameter of nanocrystals is measured by methods such as light scattering, sedimentation particle size analysis, in which solvent is used, and the measurement of the average particle diameter through direct observation of particles under an electronic microscope. Luminescent nanocrystals are prone to damage, for example from water, so it is appropriate in the present invention to use a method in which any multiple crystals are directly observed under a transmission electron microscope (TEM) or scanning electron microscope (SEM), the diameter of each crystal particle is calculated from the major-to-minor axis ratio as measured on two-dimensional projections, and the diameters are averaged. In the present invention, therefore, this method is used to calculate average particle diameters. The primary particles of luminescent nanocrystals are the single crystals nanometers to tens of nanometers in size or similar crystallites that form the nanocrystals, and the size and shape of the primary particles of luminescent nanocrystals appear to depend on the chemical makeup and structure of the primary particles and the method and parameters for the production of the primary particles.

It should be noted that during the TEM observation in the measuring method for the major and minor axes of quantum rods herein, is the longest line segment that goes across the quantum rod, and the minor axis is the shortest line segment that is perpendicular to the major axis and goes across the quantum rod.

A surface-modifying compound according to the present invention contains a group that binds to the surface of the luminescent nanocrystals and a mesogenic group in its molecule.

Since the luminescent nanocrystals have highly reactive surface atoms, they are protected with the surface-modifying compound. In addition to this, structural order of the mesogenic group itself is induced to help the nanocrystals disperse in another substance orderly.

The surface-modifying compound has one or more groups that bind to the surface of the luminescent nanocrystals per molecule. The surface-modifying compound preferably has one or more and ten or less groups that bind to the surface of the luminescent nanocrystals per molecule, more preferably one or more and eight or less, preferably one or more and six or less, preferably one or more and five or less, preferably one or more and three or less.

In the surface-modifying compound according to the present invention, it is preferred for the compound to bind to the surface of the luminescent nanocrystals that the group(s) that binds to the surface of the luminescent nanocrystals be Lewis base(s). It is more preferred that the group(s) contain one or two or more types of atoms selected from the group consisting of sulfur, nitrogen, oxygen, and phosphor, for example. Lewis base ligands are preferred because they easily coordinate to the surface of a metal

On the luminescent nanocrystals, the highly reactive surface atoms serve as coordination sites. Thus, atoms having a lone pair of electrons are preferred for the group(s) that binds to the surface of the luminescent nanocrystals. The group(s) that binds to the surface of the luminescent nanocrystals can bind to the surface of the luminescent nanocrystals wherever it is in the molecule of the surface-modifying compound, but it is preferred that the group(s) be at an end or in the middle of the surface-modifying compound, more preferably at an end of the surface-modifying compound, in light of the range of options for the surface-modifying compound. There may be one or two or more groups that bind to the surface of the luminescent nanocrystals per molecule of the surface-modifying compound.

In the surface-modifying compound according to the present invention, the group(s) in the surface-modifying compound that binds to the surface of the luminescent nanocrystals is preferably any one or more of hydroxy, thiol, carboxylic acid, amine, sulfonic acid, phosphine, phosphine oxide, and thioether. Such groups bind to metal atoms more strongly in the order of groups containing a sulfur atom, groups containing a phosphor atom, groups containing a nitrogen atom, and groups containing an oxygen atom. The order of binding strength is therefore (thiophene and thiol)>(phosphine and phosphine oxide)>(aliphatic and aromatic amines)>(hydroxy group and carboxylic acid).

This ensures that the surface-modifying compound renders the surface of the luminescent nanocrystals electrically stable and thereby protects their highly reactive surface atoms, and that the nanocrystals are in a stable form of binding with the surface-modifying compound.

The surface-modifying compound according to the present invention has a mesogenic group beside the group(s) that binds to the surface of the luminescent nanocrystals, and this helps the nanocrystals disperse orderly. “A mesogenic group” herein refers to a group capable of inducing the behavior of a liquid crystal phase, but the surface-modifying compound itself, containing a mesogenic group, does not need to exhibit a liquid crystal phase. In other words, “a mesogenic group” is a group that tends to induce structural order, and typically is a group that contains a firm moiety, such as an aromatic ring or other cyclic group. Additionally, the term “liquid crystal phase” as used here refers to a phase that has both the fluidity of liquids and anisotropy of crystals, and examples include a nematic liquid crystal, a smectic liquid crystal, and a cholesteric liquid crystal.

The mesogenic group in the surface-modifying compound according to the present invention and the molecule of the surface-modifying compound can be in any shape. Examples include rod-like, disk-like, banana-like, letter L, and letter T shapes and inclusion host structures, such as that of cyclodextrins, calixarenes, and cucurbiturils, but more preferably, they have shapes in which they can induce the behavior of a liquid crystal phase.

If having an inclusion host structure, the mesogenic group in the surface-modifying compound according to the present invention can hold a guest molecule right for its cavity size. This means, for example, the mesogenic group allows the surface-modifying compound to take in luminescent nanocrystals as a guest molecule. Moreover, given that the luminescent nanocrystals are highly reactive by virtue of having coordination sites, it is also possible to use the luminescent nanocrystals as a host molecule and bind them to a surface-modifying compound having an inclusion host structure as a guest molecule, depending on the size relationship between the surface-modifying compound and the luminescent nanocrystals.

For instance, examples of cyclodextrins that can used in the surface-modifying compound according to the present invention include α-CD (six glucose units), β-CD (seven), and γ-CD (eight) in accordance with the number of constituting glucose units, and they vary in the size of their hydrophobic cavity. If a cyclodextrin is used as the surface-modifying compound according to the present invention, it is preferred to choose one that has a cavity of a size that fits the particle diameter of the luminescent nanocrystals as a guest material. Even if the luminescent nanocrystals have a large particle diameter, cyclodextrins appear to bind to the luminescent nanocrystals with their trapezoidal-support opening by virtue of the binding sites they have for binding to the luminescent nanocrystals (hydroxy groups).

Moreover, the calixarene, which is a generic term for cyclic oligomers formed by phenols bridged together by methylene groups at positions 2 and 6, is represented by “C[n]A,” which means n phenol rings are cyclically linked together. Examples of calixarenes that can be used as the surface-modifying compound in the present invention include C[8]A and C[5]A. Calixarenes formed by four to ten cyclically linked phenol rings are preferred, and calixarenes formed by five to eight cyclically linked phenol rings are preferred.

The phenol rings that form the ring of a calixarene used in the present invention, moreover, may be unsubstituted or may have various substituents introduced thereto. For example, substituents may be introduced to the phenol rings of a calixarene to further improve the dispersion stability of the calixarene when it forms a complex with the luminescent nanocrystals. The use of a calixarene derivative that has a functional group that can serve as a ligand, such as a thiol group (—SH), introduced to an end of the phenolic rings, for example, helps form a complex even better in dispersion stability with the luminescent nanocrystals. Even if the luminescent nanocrystals have a large particle diameter, furthermore, calixarenes appear to bind to the luminescent nanocrystals with their opening by virtue of the binding sites they have for binding to the luminescent nanocrystals (hydroxy groups).

Examples of cucurbituril compounds or derivatives that can be used as the surface-modifying compound in the present invention, moreover, include cucurbit[6]uril, decamethylcucurbit[5]uril, and cucurbit[8]uril and the cucurbituril compounds or derivatives described in Japanese Unexamined Patent Application Publication No. 2001-12287. The luminescent nanocrystals as a guest molecule may be incorporated in a surface-modifying compound having an inclusion host structure, or the luminescent nanocrystals may be used as a host molecule and bound to such a surface-modifying compound as a guest molecule, depending on the size relationship between the surface-modifying compound and the luminescent nanocrystals.

These surface-modifying compounds having an inclusion host structure have a mesogenic group and group(s) that binds to the surface of the luminescent nanocrystals.

Seemingly, other inclusion host compounds, such as cryptands, cyclophanes, azacyclophanes, cyclotriveratrylenes, and their derivatives, can also be used as surface-modifying compounds like those described above.

A luminescent nanocrystal complex according to the present invention can be produced by, for example, mixing a surface-modifying compound and luminescent nanocrystals in at least one solvent and then sonicating or microwaving the solvent to remove the solvent. This gives a complex of the surface-modifying compound and the luminescent nanocrystals bound together. To the solvent, any of the surface-modifying compound and the luminescent nanocrystals can be added first, but it is preferred to add the luminescent nanocrystals to a solvent in which the surface-modifying compound, as a protecting agent, has been dispersed.

The solvent used in forming the above complex can be at least one selected from the group consisting of water; alcohols, such as methanol, ethanol, and propanol; ethylene glycols, such as monoethylene glycol, diethylene glycol, and polyethylene glycol; and ethers, such as diethyl ether, tetrahydrofuran, and diethylene glycol monomethyl ether.

It would be preferred that the mesogenic group in the surface-modifying compound according to the present invention be potent in inducing the behavior of a liquid crystal phase because it would give the surface-modifying compound a higher degree of order. The development of a liquid crystal phase involves several factors, but typically is closely linked to the mesogenic group, which is a rigid moiety, such as an aromatic ring or other cyclic group. A mesogenic group therefore refers to a group that includes a rigid moiety, for example one or more cyclic groups.

It is to be understood that “a cyclic group” herein refers to an atomic group whose constituent atoms are cyclically bound together, and cyclic groups include, for example, carbocycles, heterocycles, saturated or unsaturated ring structures, monocycles, bicyclic structures, polycyclic structures, aromatics, and non-aromatics. A cyclic group may incorporate at least one heteroatom and may further be substituted with at least one substituent (reactive functional group or organic group (alkyl, aryl, etc.).

The lower limit for the number of cyclic groups in a mesogenic group according to the present invention is preferably 1 or more, preferably 2 or more, preferably 2 or more, preferably 3 or more, preferably 4 or more. The upper limit for the number of cyclic groups is preferably 15 or less, preferably 10 or less, preferably 8 or less, preferably 7 or less, preferably 6 or less, preferably 5 or less, preferably 4 or less.

Two or more and fifteen or less cyclic rings provide greater interactions with cyclic compounds.

If the surface-modifying compound according to the present invention has a rod-like, letter L, letter T, or cruciform shape, preferred examples of surface-modifying compounds may include those represented by general formula (i) below.

That is, the surface-modifying compound according to the present invention is preferably general formula (i).

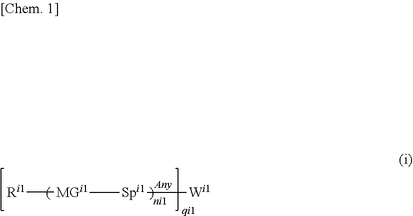

“In general formula (i) above,

MGⁱ¹ represents a mesogenic group,

SPⁱ¹ represents a single bond or spacer group,

Rⁱ¹ represents a hydrogen atom, halogen atom, cyano group, or C1-18 linear or branched alkyl group, where one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —CC—, —NH—, —PH—, or —POH—, and the hydrogen atom, halogen atom, cyano group, or one or more hydrogen atoms in the alkyl group may be substituted with general formula (i-1),

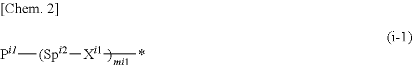

. . . represents . . . (in general formula (i-1) above, Pⁱ¹ represents a reactive functional group,

Spⁱ² represents a single bond or C1-18 alkylene group, where the hydrogen atoms in the alkylene group may be substituted with one or more halogen atoms or CNs, and one CH₂ group present in the alkylene group, or each of nonadjacent two or more independently, may be substituted with —O—, —COO—, —OCO—, or —OCO—O—,

Xⁱ¹ represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P-Spⁱ² and Spⁱ²-X, however, include no —O—O—, —O—NH—, —S—S—, or —O—S— group.), mi1 represents 0 or 1, and * represents a bond.)

Wⁱ¹ represents a monovalent to tetravalent functional group, specifically —SH, —PH₂, —PH—, —POH₂, —POH—, —NH₂, —NH—, —OH, —COOH, a group represented by general formula (W-1) to (W-14), or a single bond.

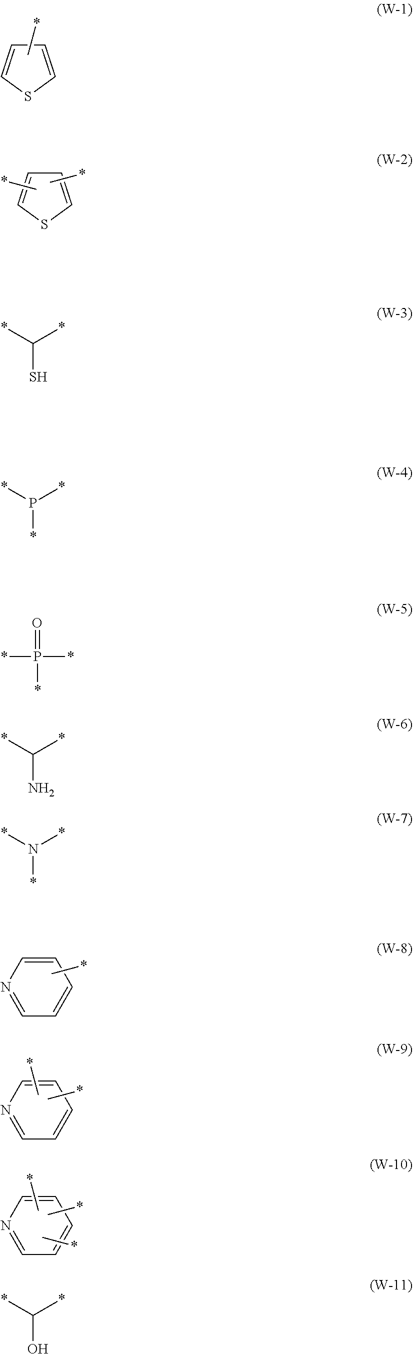

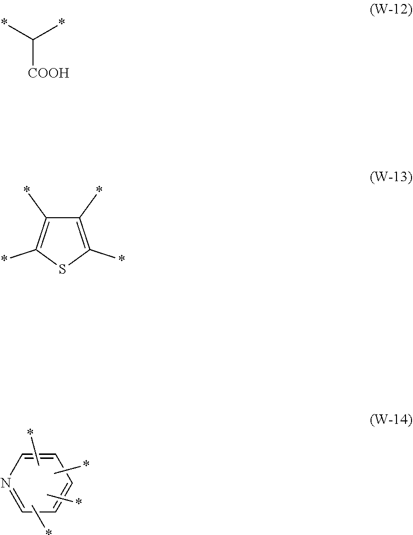

qi1 represents an integer of 1 to 4, and if qi1 is 2 or more and there are multiple Rⁱ¹s, MGⁱ¹s, or SPⁱ¹s, they may be the same or different,

ni1 represents an integer of 0 to 8, and if ni1 is 2 or more and there are multiple MGⁱ¹s or SPⁱ¹s, they may be the same or different,

if Wⁱ¹ is a single bond, qi1 is 2, and

if -Any-Wⁱ¹ is a divalent to tetravalent functional group, the corresponding qi1 represents an integer of 2 to 4, and * represents a bond.)”

A luminescent nanocrystal complex according to the present invention includes luminescent nanocrystals and a surface-modifying compound (or ligand) that modifies the surface of the luminescent nanocrystals, and the ligand has a mesogenic group that has a structure as described above and a binding site for binding to the luminescent nanocrystals. The resulting uniform dispersion of the luminescent nanocrystals improves their efficiency in light emission. The durability of the phosphor is also improved.

If a quantum-rod phosphor is used as the luminescent nanocrystals, moreover, the complex improves in alignment and achieves a higher degree of polarization of the light it emits.

In general formula (i) above, Wⁱ¹, which is a monovalent to tetravalent functional group, represents —SH, —PH₂, —PH—, —POH₂, —POH—, —NH₂, —NH—, —OH, —COOH, a group represented by general formulae (W-1) to (W-14) above, or a single bond.

In general formula (i) above, Wⁱ¹ is preferably a group that binds to the surface of the luminescent nanocrystals, preferably —SH, —PH₂, —PH—, —POH₂, —POH—, —NH₂, —NH—, —OH, —COOH, or a group represented by general formulae (W-1) to (W-12), more preferably —PH—, —POH—, —NH—, —COOH, or the group represented by formula (W-1), (W-3), (W-5), (W-6), (W-8), (W-11), or (W-12). Any-Wⁱ¹ in the formula means multivalency.

In general formula (i) above, if Wⁱ¹ is —SH, —PH₂, —POH₂, —NH₂, —OH, —COOH, or general formula (W-1) or (W-8) (if Wⁱ¹ is a monovalent organic group), qi1=1. This is a form in which the surface-modifying compound has a group that binds to the surface of the luminescent nanocrystals at least at its end.

In general formula (i) above, if Wⁱ¹ is —PH—, —POH—, —NH—, or general formula (W-2), (W-3), (W-6), (W-9), (W-11), or (W-12) (if Wⁱ¹ is a divalent organic group), qi1=2. This is a form in which the surface-modifying compound has at least one group that binds to the surface of the luminescent nanocrystals except at its end. In this form, therefore, the surface-modifying compound tends to be L-shaped with Wⁱ¹ at its center.

In general formula (i) above, if Wⁱ¹ is general formula (W-4), (W-5), (W-7), or (W-10) (if Wⁱ¹ is a trivalent organic group), qi1=3. This is a form in which the surface-modifying compound has at least one group that binds to the surface of the luminescent nanocrystals except at its end. In this form, the surface-modifying compound tends to be T-shaped with Wⁱ¹ at its center.

In general formula (i) above, if Wⁱ¹ is general formula (W-13) or (W-14) (if Wⁱ¹ is a tetravalent organic group, qi1=4. This is a form in which the surface-modifying compound has at least one group that binds to the surface of the luminescent nanocrystals except at its end. In this form, the surface-modifying compound tends to be cruciform with Wⁱ¹ at its center.

In general formula (i) above, if Wⁱ¹ is a single bond, qi1 is 2, and the structure of general formula (A-5), given later herein, is preferred.

In this case, there are multiple Rⁱ¹s, MGⁱ¹s, or SPⁱ¹s, and, as stated above, Rⁱ¹s, MGⁱ¹s, or SPⁱ¹s may be the same or different. Thus, they are expressed as R^{i1′}, MG^{i1′}, or SP^{i1′} in the foregoing.

In general formula (i) above, ni1 represents an integer of 0 to 8, and if ni1 is 2 or more and there are multiple MGⁱ¹s or SPⁱ¹s, they may be the same or different. The lower limit for ni1 is preferably 1, more preferably 2, even more preferably 3. The upper limit for ni1 is preferably 8, more preferably 7, even more preferably 6.

In general formula (i) above, qi1 represents an integer of 1 to 4. qi1 is preferably an integer of 1 to 3, more preferably an integer of 1 or 2.

In general formula (i) above, a preferred Rⁱ¹ is a hydrogen atom, halogen atom, cyano group, or C1-18 linear or branched alkyl group (The alkyl group may be linear or branched, and one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—.), or a group represented by general formula (i-1). A more preferred Rⁱ¹ is a hydrogen atom, halogen atom, cyano group, or C1-10 linear or branched alkyl group (The alkyl group may be linear or branched, and one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—.) or a group represented by general formula (i-1).

Examples of alkyl groups herein include methyl, ethyl, propyl, butyl, pentyl, hexyl, heptyl, octyl, nonyl, decyl, isopropyl, and isobutyl groups. Examples of alkylene groups herein include methylene, ethylene, propylene, butylene, hexylene, and octylene groups.

In general formula (i-1) above, Pⁱ¹, as a reactive functional group, preferably represents a substituent selected from the polymerizable groups represented by formulae (P-1) to (P-20) below, more preferably a group represented by formulae (P-1) to (P-19). * represents a bond.

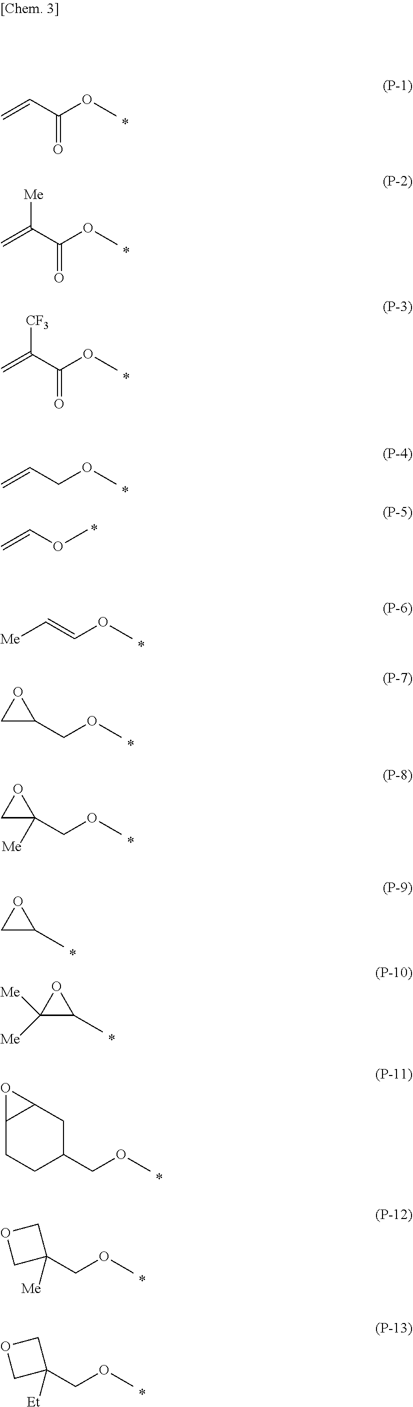

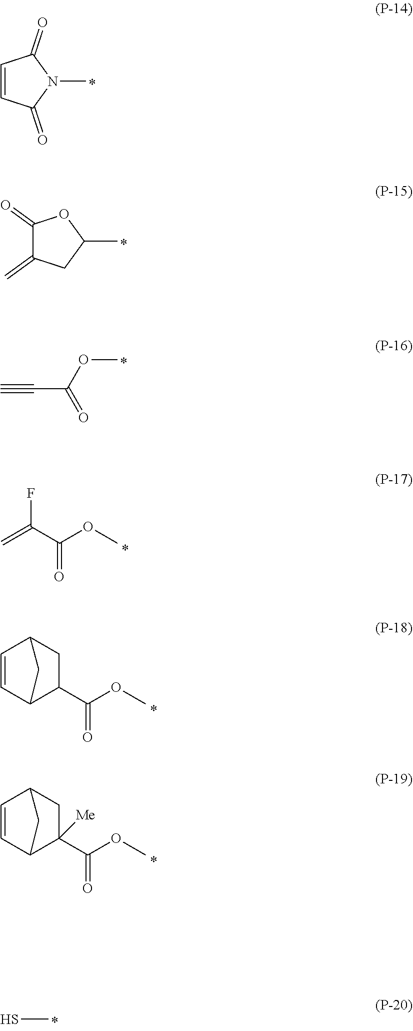

Especially because of the relationship to other compounds (e.g., a binder resin), it is preferred that Pⁱ¹ be formula (P-1), (P-2), (P-4), (P-5), (P-7), (P-9), (P-11), (P-12), (P-13), or (P-15), in particular formula (P-1), (P-2), (P-4), (P-5), (P-7), (P-12), or (P-13).

In general formula (i-1) above, a preferred Xⁱ¹ is —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —CF═CF—, —C≡C—, or a single bond (P-Sp₃ and Sp₃-X, however, include no —O—O—, —O—NH—, —S—S—, or —O—S— group.). A more preferred Xⁱ¹ is —O—, —S—, —OCH₂—, —CH₂O—, —CO—, —COO—, —OCO—, —SCH₂—, —CH₂S—, —CF₂O—, —OCF₂—, —CF₂S—, —SCF₂—, —COO—CH₂CH₂—, —OCO—CH₂CH₂—, —CH₂CH₂—COO—, —CH₂CH₂—OCO—, —CF═CF—, —C≡C—, or a single bond. (Pⁱ¹-Spⁱ² and Spⁱ²-Xⁱ¹, however, include no —O—O—, —O—NH—, —S—S—, or —O—S— group.)

In general formula (i-1) above, a preferred Spⁱ² is preferably a single bond or C1-18 alkylene group (the hydrogen atoms in the alkylene group may be substituted with one or more halogen atoms, and one CH₂ group present in this group, or each of nonadjacent two or more independently, may be substituted with —O—, —COO—, —OCO—, or —OCO—O—). A more preferred Spⁱ² is a single bond or C2-12 alkylene group (the hydrogen atoms in the alkylene group may be substituted with one or more halogen atoms, and one CH₂ group present in this group, or each of nonadjacent two or more independently, may be substituted with —O—, —COO—, —OCO—, or —OCO—O—).

In general formula (i-1) above, mi1 is preferably 0 or 1, in particular 1.

In general formula (i) above, SPⁱ¹ is preferably a spacer group that is a divalent organic group.

A divalent organic group refers to a group whose chemical structure is formed by the conversion of an organic compound into a divalent group, or an atomic group that results from removing two hydrogen atoms from an organic compound.

In general formula (i) above, SPⁱ¹ preferably represents is preferably a single bond or C1-18 alkylene group (The hydrogen atoms in the alkylene group may be substituted with one or more halogen atoms or CNs, and one CH₂ group present in this group, or each of nonadjacent two or more independently, may be substituted with —O—, —COO—, —OCO—, or —OCO—O—.) and more preferably represents is preferably a single bond or C1-10 alkylene group (The hydrogen atoms in the alkylene group may be substituted with one or more halogen atoms or CNs, and one CH₂ group present in this group, or each of nonadjacent two or more independently, may be substituted with —O—, —COO—, —OCO—, or —OCO—O—.).

In general formula (i) above, MGⁱ¹ is a mesogenic group, preferably a divalent organic group incorporating a cyclic group.

A divalent organic group incorporating a cyclic group refers to a divalent-group form of an organic compound that incorporates a cyclic group, the cyclic group being an atomic group whose constituent atoms are cyclically bound together, or an atomic group resulting from removing two hydrogen atoms from an organic compound that incorporates a cyclic group.

In general formula (i) above, MGⁱ¹ is more preferably represented by general formula (i-2) below

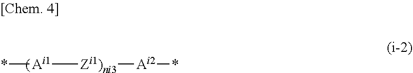

(In general formula (i-5) above, Aⁱ¹ and Aⁱ² each independently represent one ring structure selected from the group consisting of unsubstituted or substituted 1,4-phenylene, 1,4-cyclohexylene, 1,4-cyclohexenyl, tetrahydropyran-2,5-diyl, 1,3-dioxan-2,5-diyl, tetrahydrothiopyran-2,5-diyl, thiophen-2,5-diyl, 1,4-bicyclo(2,2,2)octylene, decahydronaphthalen-2,6-diyl, pyridin-2,5-diyl, pyrimidin-2,5-diyl, pyrazin-2,5-diyl, thiophen-2,5-diyl-, 1,2,3,4-tetrahydronaphthalen-2,6-diyl, 2,6-naphthylene, phenanthren-2,7-diyl, 9,10-dihydrophenanthren-2,7-diyl, 1,2,3,4,4a,9,10a-octahydrophenanthren-2,7-diyl, 1,4-naphthylene, benzo[1,2-b:4,5-b′]dithiophen-2,6-diyl, benzo[1,2-b:4,5-b′]diselenophen-2,6-diyl, [1]benzothieno[3,2-b]thiophen-2,7-diyl, [1]benzoselenopheno[3,2-b]selenophen-2,7-diyl, and fluoren-2,7-diyl groups,

the substitution of one or more or two or more hydrogen atoms in the ring structures may be substituted with a fluorine or chlorine atom, a CF₃, OCF₃, CN, nitro, amino, carboxyl, hydroxy, aldehyde, mercapto, carbamoyl, sulfo, thienyl, pyridyl, C1-8 alkyl, C1-8 alkoxy, C1-8 alkanoyl, C1-8 alkanoyloxy, C2-8 alkenyl, C2-8 alkenyloxy, C1-8 alkenoyl, or C1-8 alkenoyloxy group, or a substituent represented by general formula (i-1) above, where the C1-8 alkyl, C1-8 alkoxy, C1-8 alkanoyl, C1-8 alkanoyloxy, C2-8 alkenyl, C2-8 alkenyloxy, C1-8 alkenoyl, and C1-8 alkenoyloxy groups and substituent represented by general formula (i-1) may be further substituted with a fluorine or chlorine atom or a CF₃, OCF₃, CN, nitro, amino, carboxyl, hydroxy, aldehyde, mercapto, carbamoyl, sulfo, thienyl, or pyridyl group,

Zⁱ¹ represents —COO—, —OCO—, —CO—S—, —S—CO—, —O—CO—O—, —CH₂CH₂—, —OCH₂—, —CH₂O—, —OCF₂—, —OCF₂—, —CF₂S—, —SCF₂—, —CH═CH—, —CF═CF—, —C≡C—, —CH═CHCOO—, —OCOCH═CH—, —CH₂CH₂COO—, —CH₂CH₂OCO—, —COOCH₂CH₂—, —OCOCH₂CH₂—, —CONH—, —NHCO—, —N═N—, —CH═N—N═CH—, a halogenated or non-halogenated C2-10 alkyl group, or a single bond,

ni3 represents an integer of 1 to 4, and if ni3 is 2 or more and there are multiple Aⁱ¹s and Zⁱ¹s, they may be the same or different, and * represents a bond.)

In general formula (i-2) above, each of Aⁱ¹ and Aⁱ² is preferably independently one ring structure selected from the group consisting of 1,4-phenylene, 1,4-cyclohexylene, 1,4-cyclohexenyl, 1,3-dioxan-2,5-diyl, 1,4-bicyclo(2,2,2)octylene, decahydronaphthalen-2,6-diyl, pyridin-2,5-diyl, pyrimidin-2,5-diyl, pyrazin-2,5-diyl, thiophen-2,5-diyl-, 1,2,3,4-tetrahydronaphthalen-2,6-diyl, 2,6-naphthylene, phenanthren-2,7-diyl, 9,10-dihydrophenanthren-2,7-diyl, 1,2,3,4,4a,9,10a-octahydrophenanthren-2,7-diyl, 1,4-naphthylene, benzo[1,2-b:4,5-b′]dithiophen-2,6-diyl, benzo[1,2-b:4,5-b′]diselenophen-2,6-diyl, [1]benzothieno[3,2-b]thiophen-2,7-diyl, and [1]benzoselenopheno[3,2-b]selenophen-2,7-diyl groups unsubstituted or substituted with the substituent(s) (Sub) specified below. One or two or more hydrogen atoms in the ring structures may be substituted, for example with the substituent(s) (Sub) specified below.

The substituent(s) (Sub) is at least one selected from the group consisting of fluorine and chlorine atoms, CF₃, OCF₃, CN, nitro, amino, phosphine, phosphonic acid, carboxyl, hydroxy, aldehyde, mercapto, carbamoyl, sulfo, thienyl, pyridyl, C1-8 alkyl, C1-8 alkoxy, C1-8 alkanoyl, C1-8 alkanoyloxy, C2-8 alkenyl, C2-8 alkenyloxy, C1-8 alkenoyl, and C1-8 alkenoyloxy groups, and a substituent represented by general formula (i-1) above, with the proviso that the C1-8 alkyl, C1-8 alkoxy, C1-8 alkanoyl, C1-8 alkanoyloxy, C2-8 alkenyl, C2-8 alkenyloxy, C1-8 alkenoyl, and C1-8 alkenoyloxy groups and substituent represented by general formula (i-1) may be substituted with a fluorine or chlorine atom or a CF₃, OCF₃, CN, nitro, amino, phosphine, phosphonic acid, carboxyl, hydroxy, aldehyde, mercapto, carbamoyl, sulfo, thienyl, or pyridyl group.

Aⁱ¹ and Aⁱ² may be the same or different, and if ni3 is 2 or more and there are multiple Aⁱ¹s, they may be the same or different.

In general formula (i-2) above, Zⁱ¹ is preferably —COO—, —OCO—, —CH₂CH₂—, —OCH₂—, —CH₂O—, —OCF₂—, —OCF₂—, —CF₂S—, —SCF₂—, —CH═CH—, —CF═CF—, —C≡C—, —CH₂CH₂COO—, —CH₂CH₂OCO—, —COOCH₂CH₂—, —OCOCH₂CH₂—, a halogenated or non-halogenated C2-10 alkylene group, or a single bond.

If ni3 is 2 or more and there are multiple Zⁱ¹s, they may be the same or different.

In general formula (i-2) above, ni3 preferably represents an integer of 1 to 3.

In general formula (i) according to the present invention, a preferred specific form of MGⁱ¹ (s) is at least one selected from the group consisting of general formulae (N-1) to (N-21), (M-1) to (M-18), (K-1) to (K-6), (L-1) to (L-13), (RM-1) to (RM-25), and (U-1) to (U-50) below.

In this preferred specific form of MGⁱ¹(s), * represents a bond.

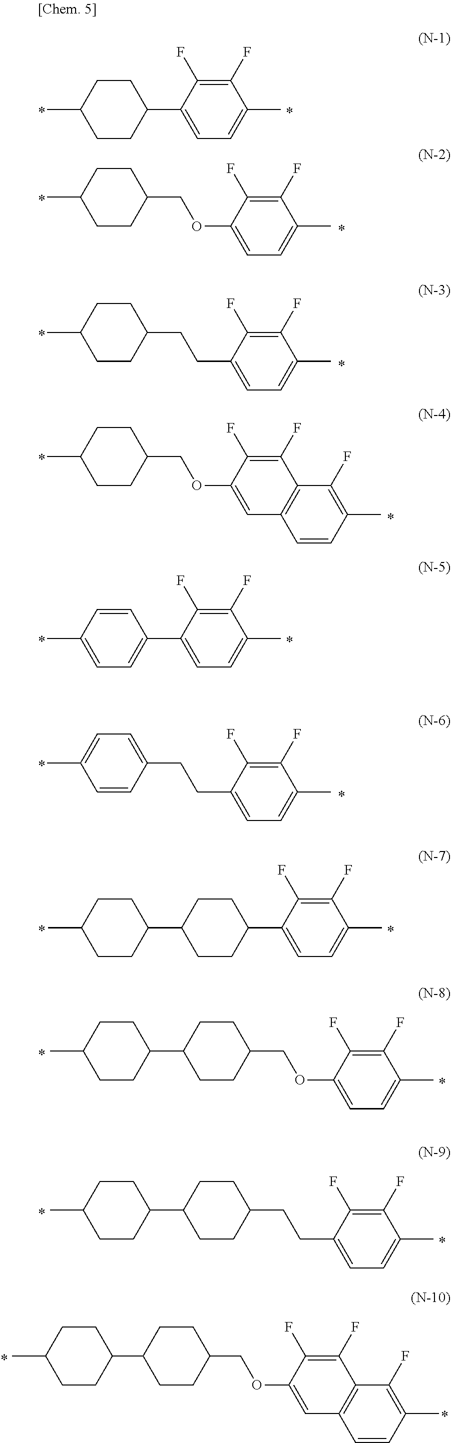

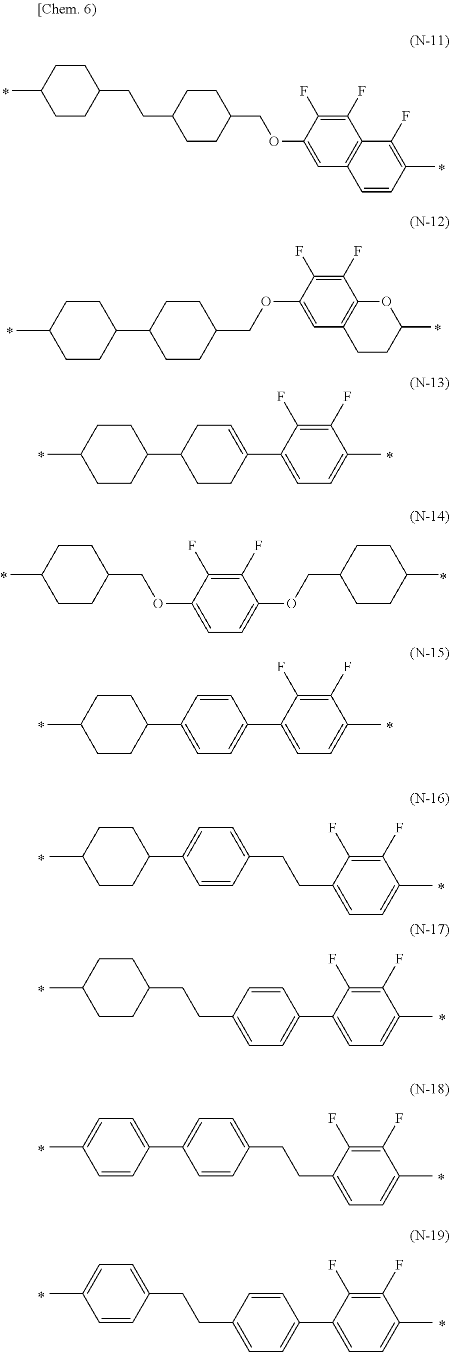

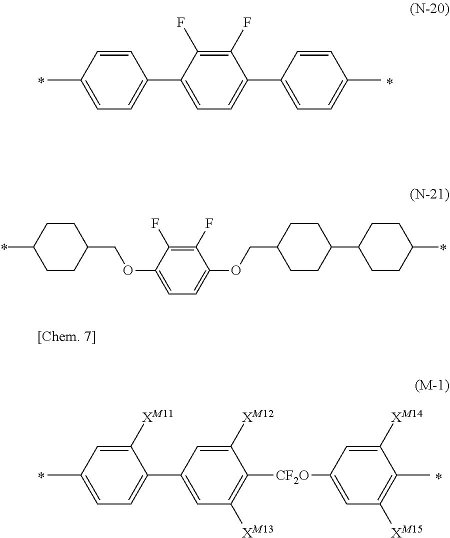

(where X^{M11} to X^{M15} each independently represent a hydrogen or fluorine atom.)

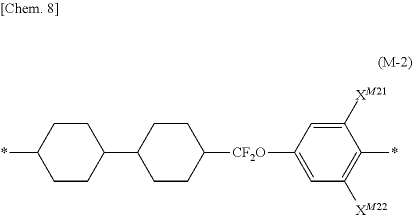

(where X^{M21} and X^{M22} each independently represent a hydrogen or fluorine atom.)

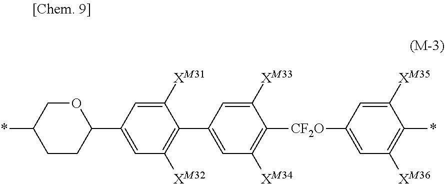

(where X^{M31} to X^{M36} each independently represent a hydrogen or fluorine atom.)

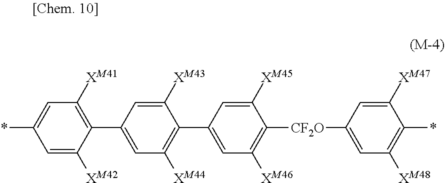

(where X^{M41} to X^{M48} each independently represent a fluorine or hydrogen atom.)

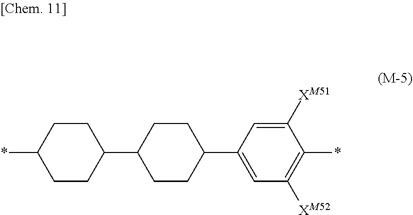

(where X^{M51} and X^{M52} each independently represent a hydrogen or fluorine atom.)

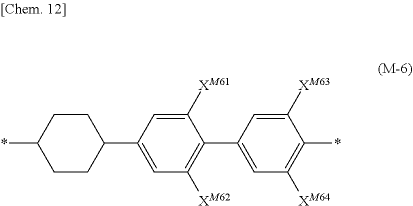

(where X^{M61} to X^{M64} each independently represent a fluorine or hydrogen atom.)

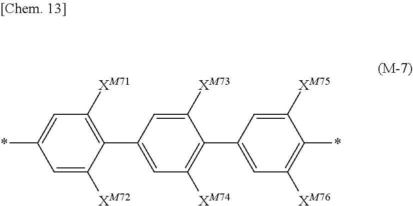

(where X^{M71} to X^{M76} each independently represent a fluorine or hydrogen atom, and R^{M71} represents a C1-5 alkyl, C2-5 alkenyl, or C1-4 alkoxy group.)

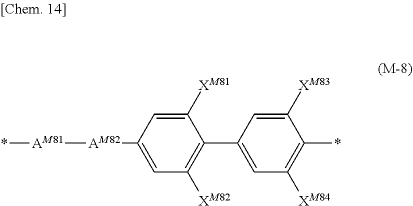

(where X^{M81} to X^{M84} each independently represent a fluorine or hydrogen atom, Y represents a fluorine atom, chlorine atom, or —OCF₃, and AM8¹ and A^{M82} each independently represent a 1,4-cyclohexylene group, 1,4-phenylene group, or

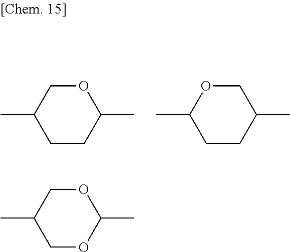

with the proviso that hydrogen atom(s) on the 1,4-phenylene group may be substituted with a fluorine atom.)

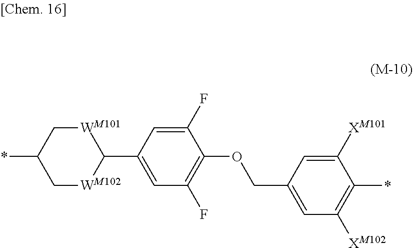

(where X^{M101} and X^{M102} each independently represent a fluorine or hydrogen atom, and W^{M101} and W^{M102} each independently represent —CH₂— or —O—.)

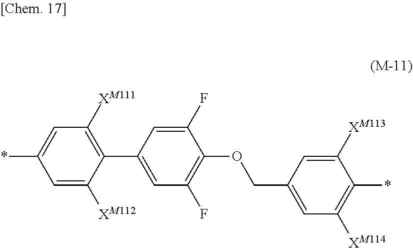

(where X^{M111} to X^{M114} each independently represent a fluorine or hydrogen atom.)

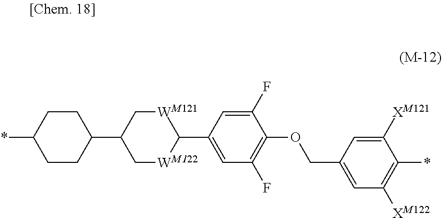

(where X^{M121} and X^{M122} each independently represent a fluorine or hydrogen atom, and W^{M121} and W^{M122} each independently represent —CH₂— or —O—.)

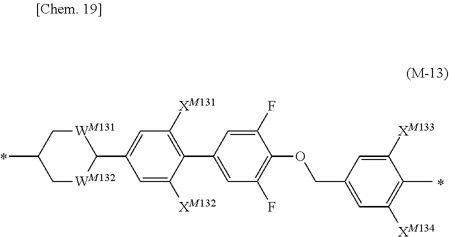

(where X^{M131} to X^{M134} each independently represent a fluorine or hydrogen atom, and W^{M131} and W^{M132} each independently represent —CH₂— or —O—.)

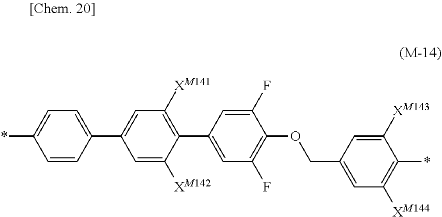

(where X^{M141} to X^{M144} each independently represent a fluorine or hydrogen atom.)

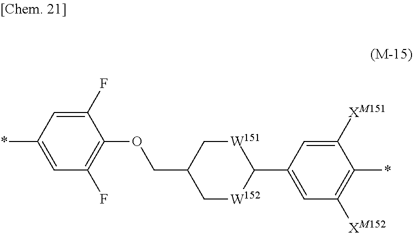

(where X^{M151} and X^{M152} each independently represent a fluorine or hydrogen atom, and W^{M151} and W^{M152} each independently represent —CH₂— or —O—.)

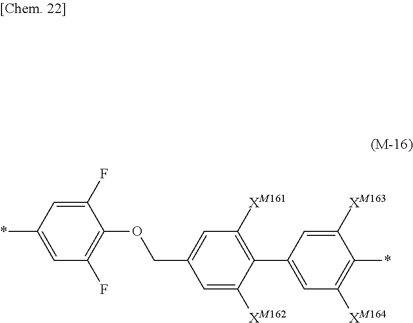

(where X^{M161} to X^{M164} each independently represent a fluorine or hydrogen atom.)

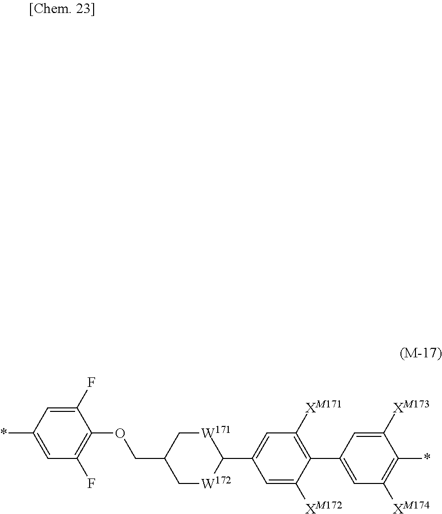

(where X^{M171} to X^{M174} each independently represent a fluorine or hydrogen atom, and W^{M171} and WM^{M172} each independently represent —CH₂— or —O—.)

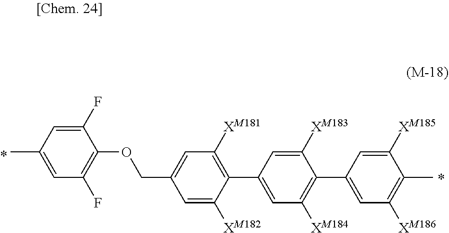

(where X^{M181} to X^{M186} each independently represent a fluorine or hydrogen atom.)

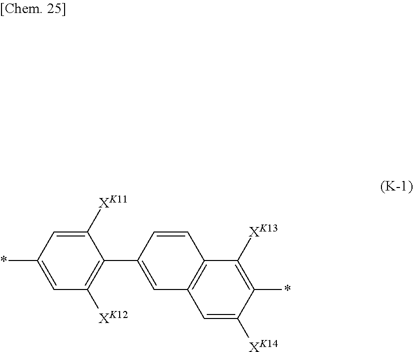

(where X^{K11} to X^{K14} each independently represent a hydrogen or fluorine atom.)

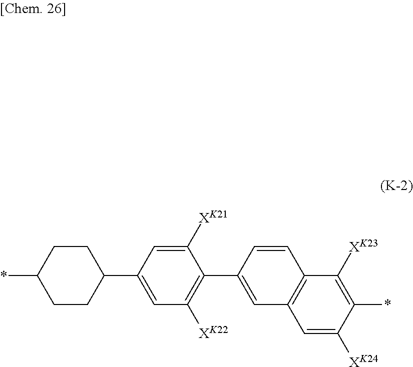

(where X^{K21} to X^{K24} each independently represent a hydrogen or fluorine atom.)

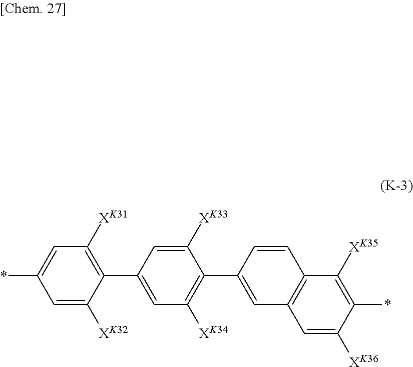

(where X^{K31} to X^{K36} each independently represent a hydrogen or fluorine atom.)

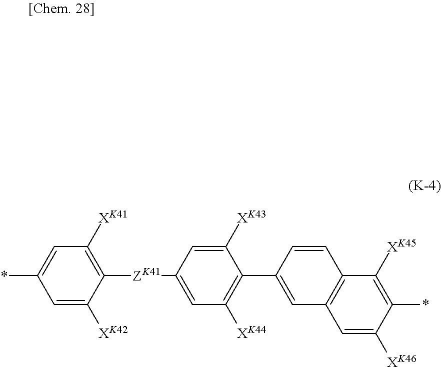

(where X^{K41} to X^{K46} each independently represent a hydrogen or fluorine atom, Z^{K41} represents —OCH₂—, —CH₂O—, —OCF₂—, or —CF₂O—.)

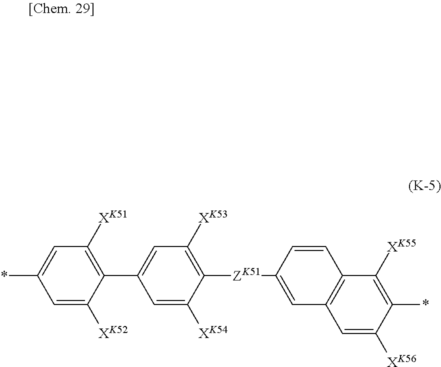

(where X^{K51} to X^{K56} each independently represent a hydrogen or fluorine atom, and Z^{K51} represents —OCH₂—, —CH₂O—, —OCF₂—, or —CF₂O—.)

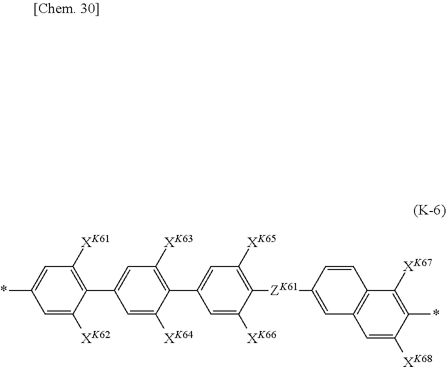

(where X^{K61} to X^{K68} each independently represent a hydrogen or fluorine atom, and Z^{K61} represents —OCH₂—, —CH₂O—, —OCF₂—, or —CF₂O—.)

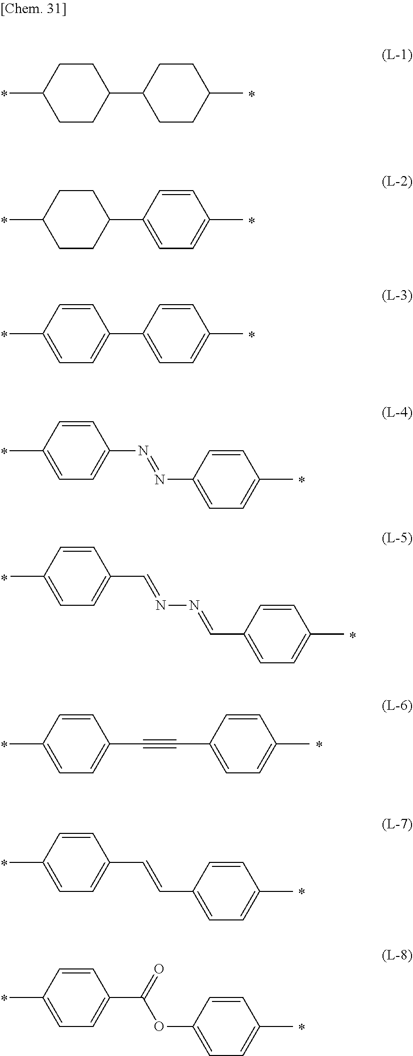

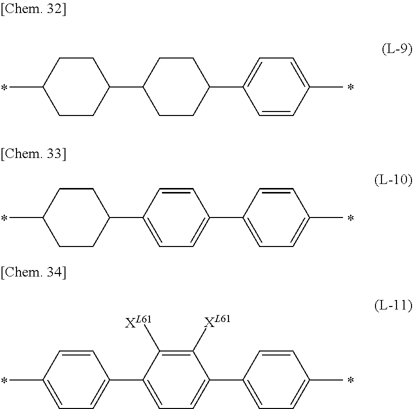

(where X^{L61} and X^{L62} each independently represent a hydrogen or fluorine atom.)

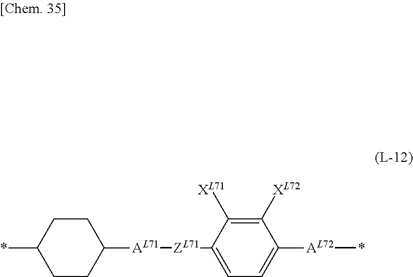

(where A^{L71} and A^{L72} each independently represent the same meaning as A^{M81} in general formula (M-8), with the proviso that the hydrogen atoms on A^{L71} and A^{L72} may each independently be substituted with a fluorine atom, Z^{L71} represents the same meaning as Z^{K41} in general formula (K-4), and X^{L71} and X^{L72} each independently represent a fluorine or hydrogen atom.)

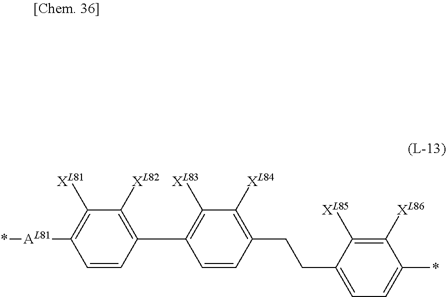

(where A^{L81} represents the same meaning as A^{M81} in general formula (M-8) or a single bond, with the proviso that the hydrogen atoms on A^{L81} may each independently be substituted with a fluorine atom, and X^{L81} to X^{L86} each independently represent a fluorine or hydrogen atom.)

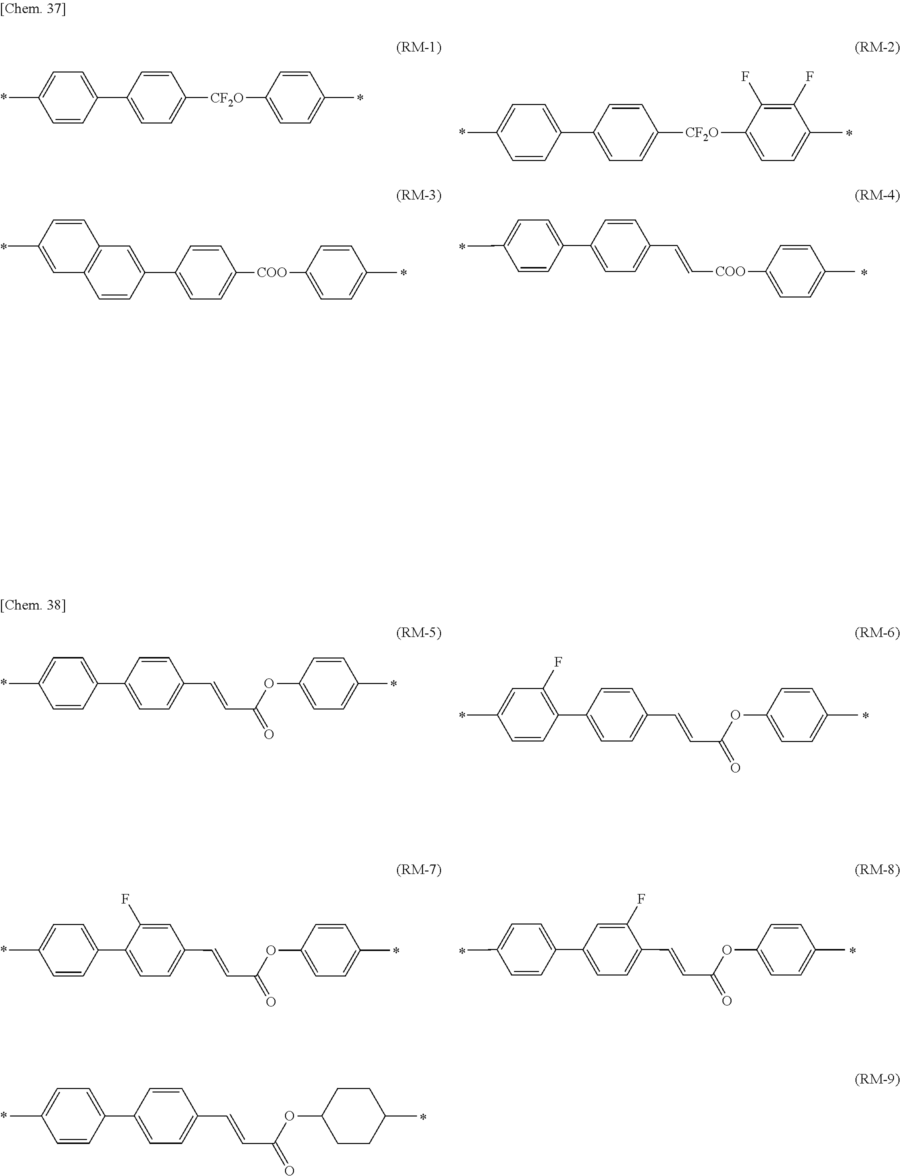

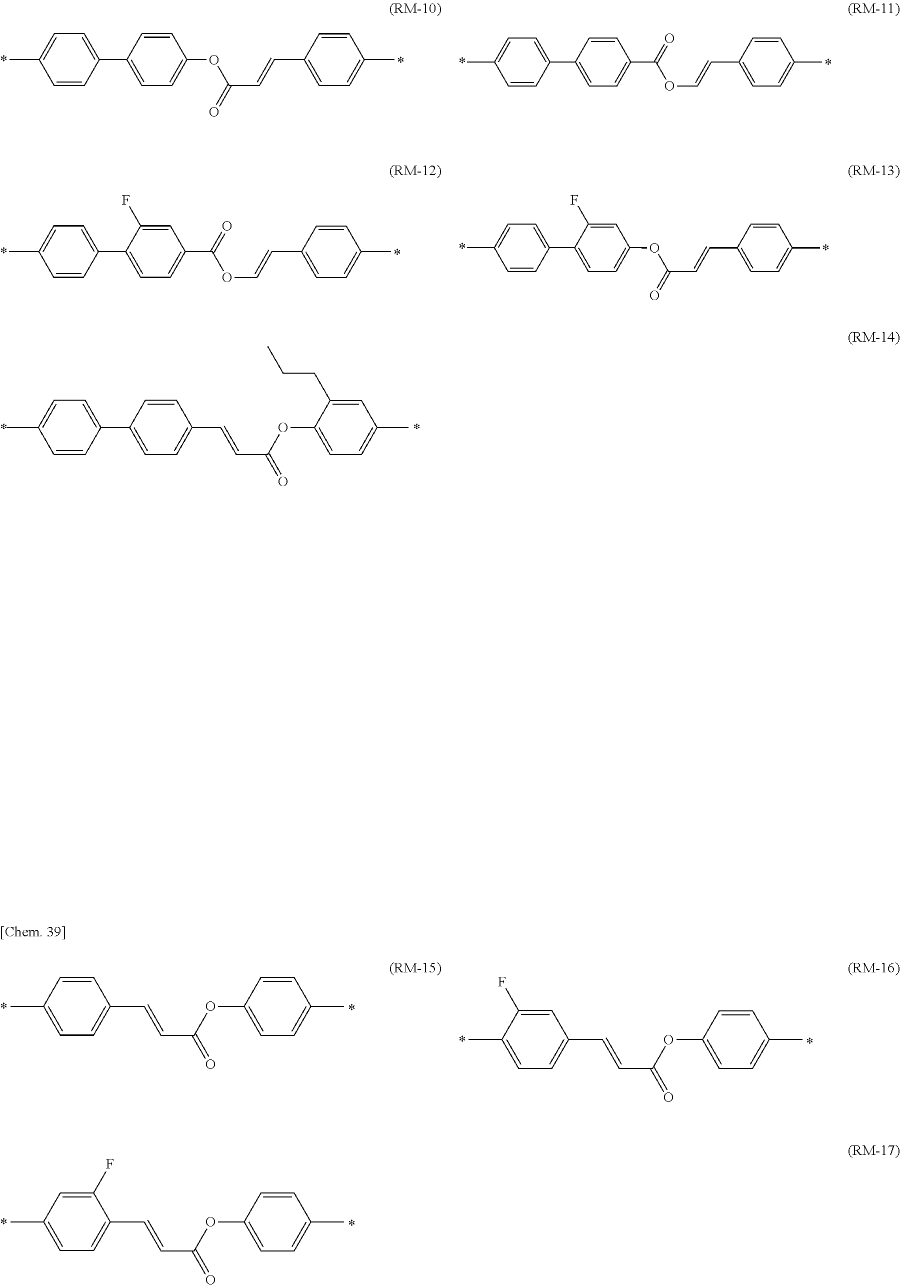

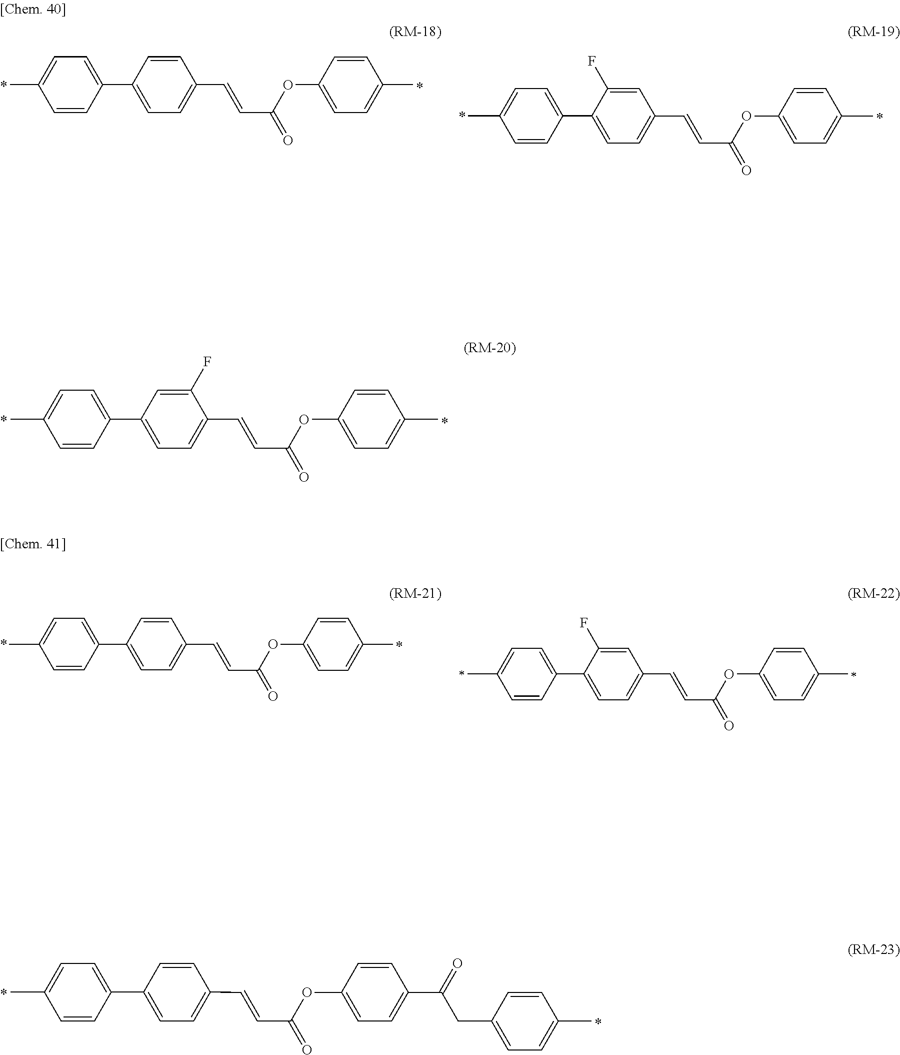

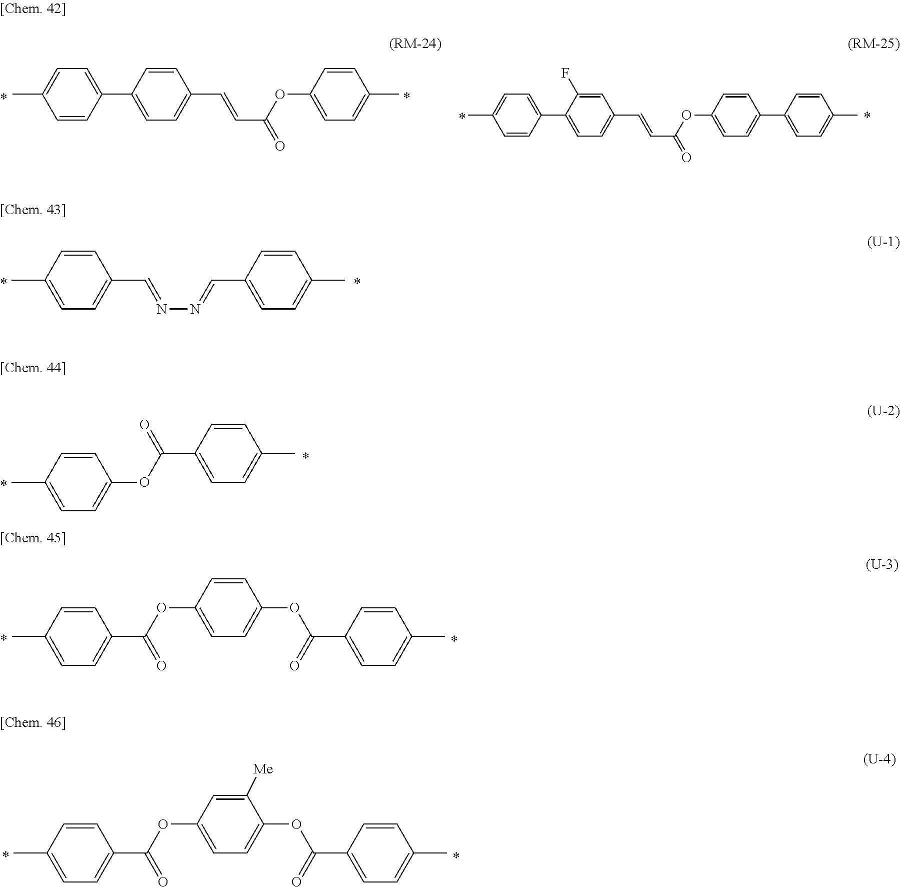

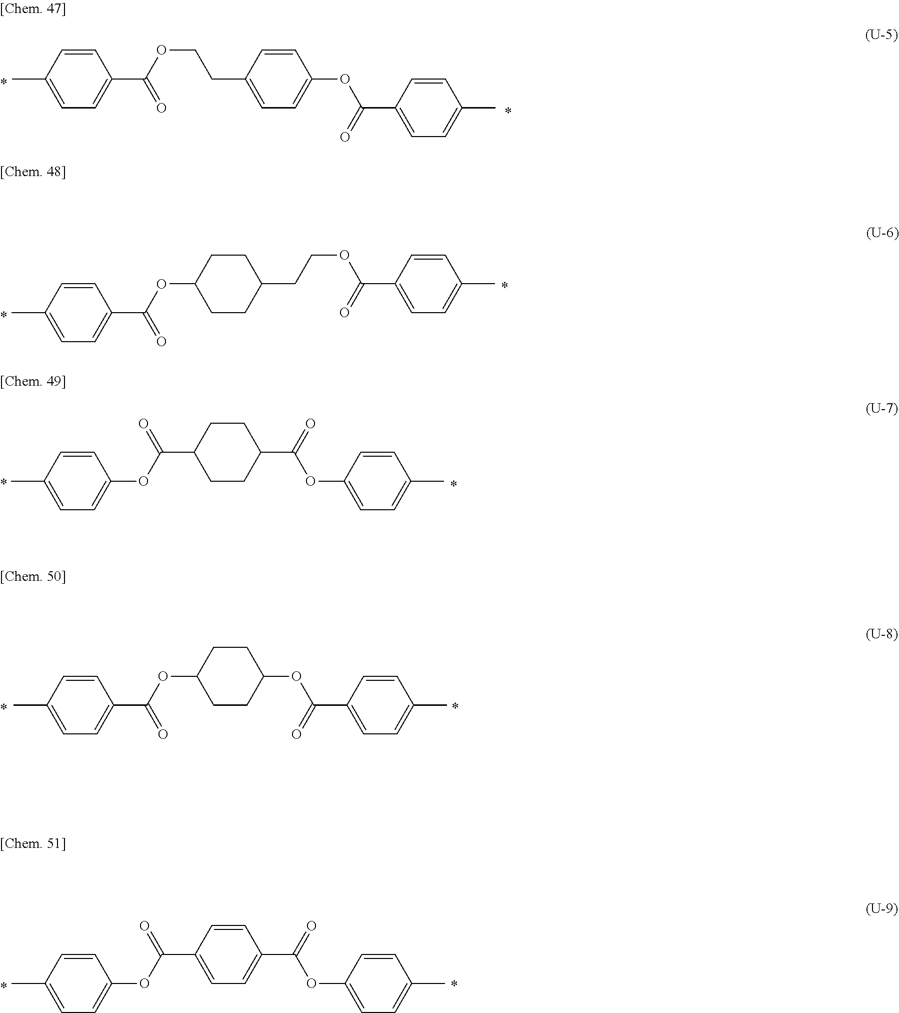

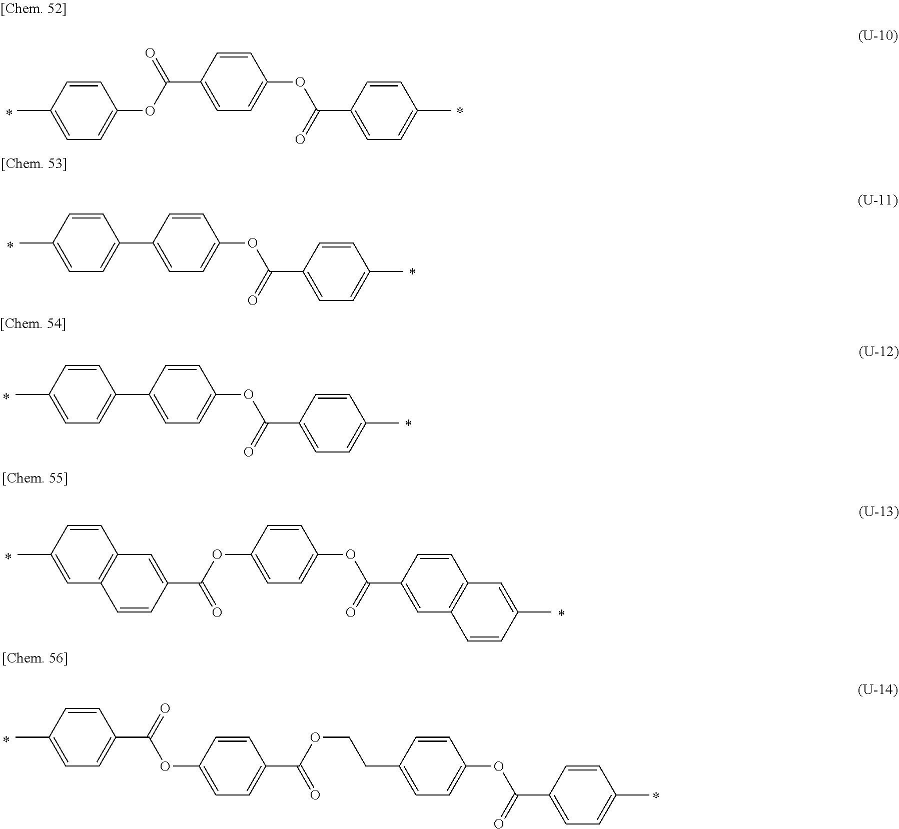

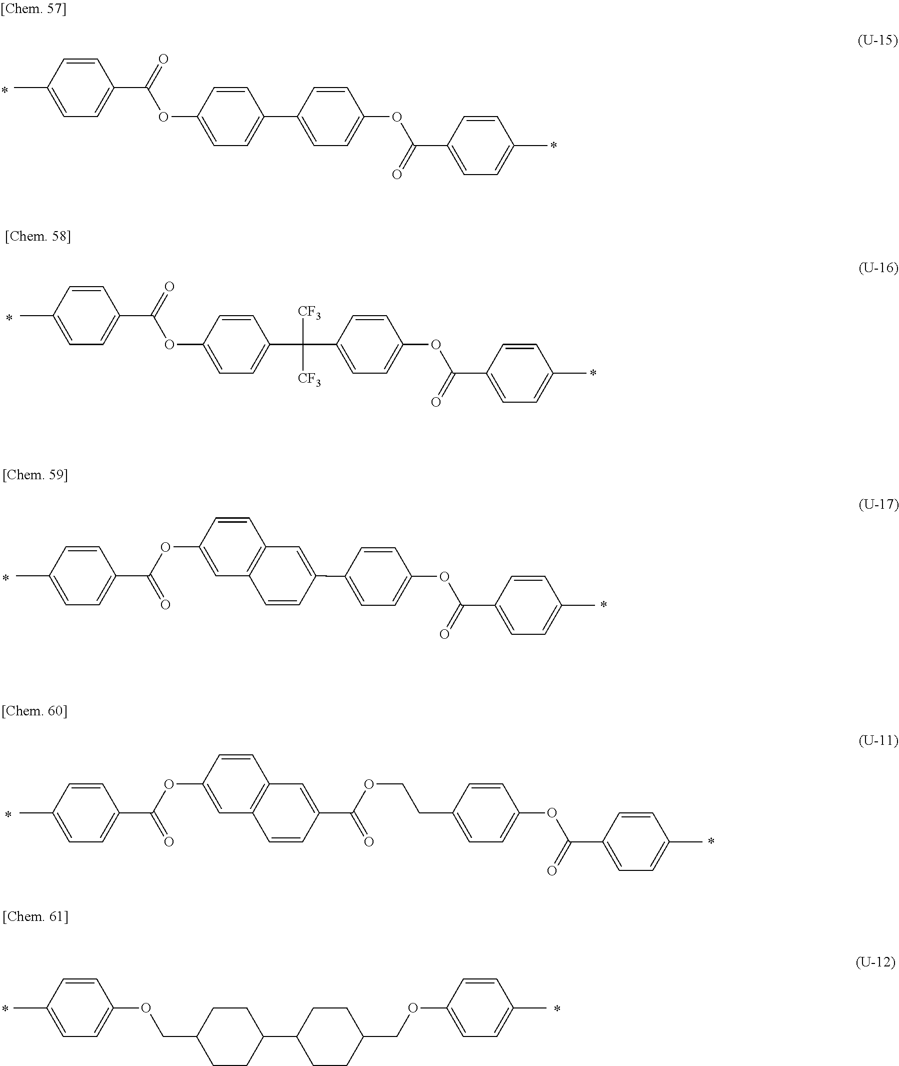

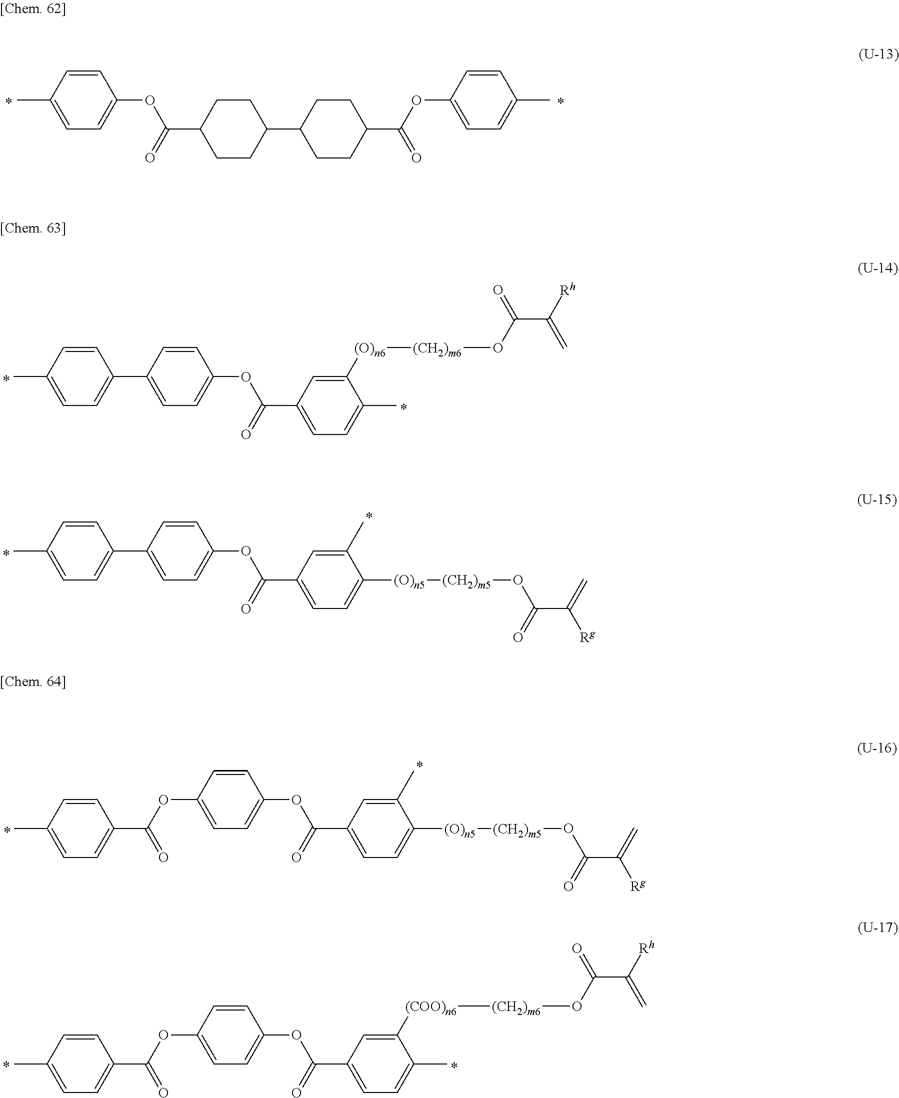

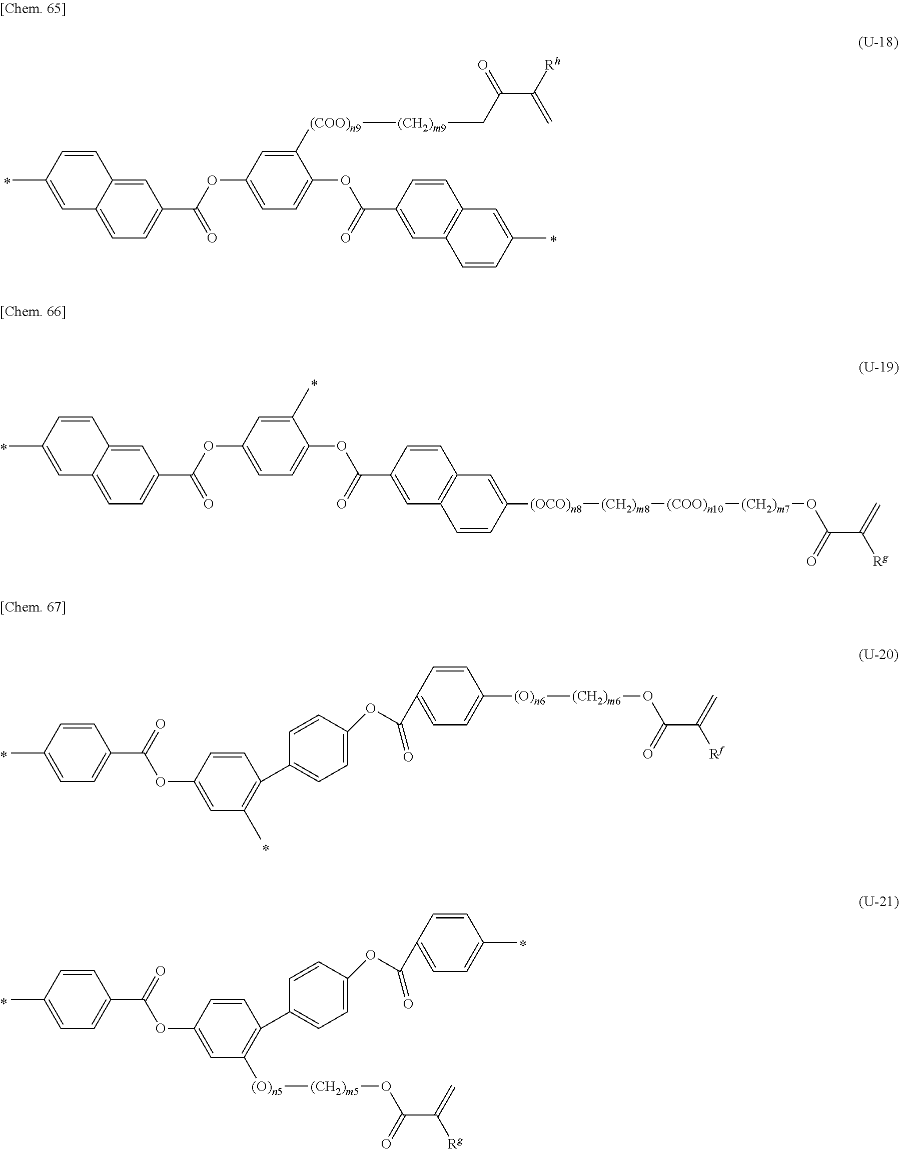

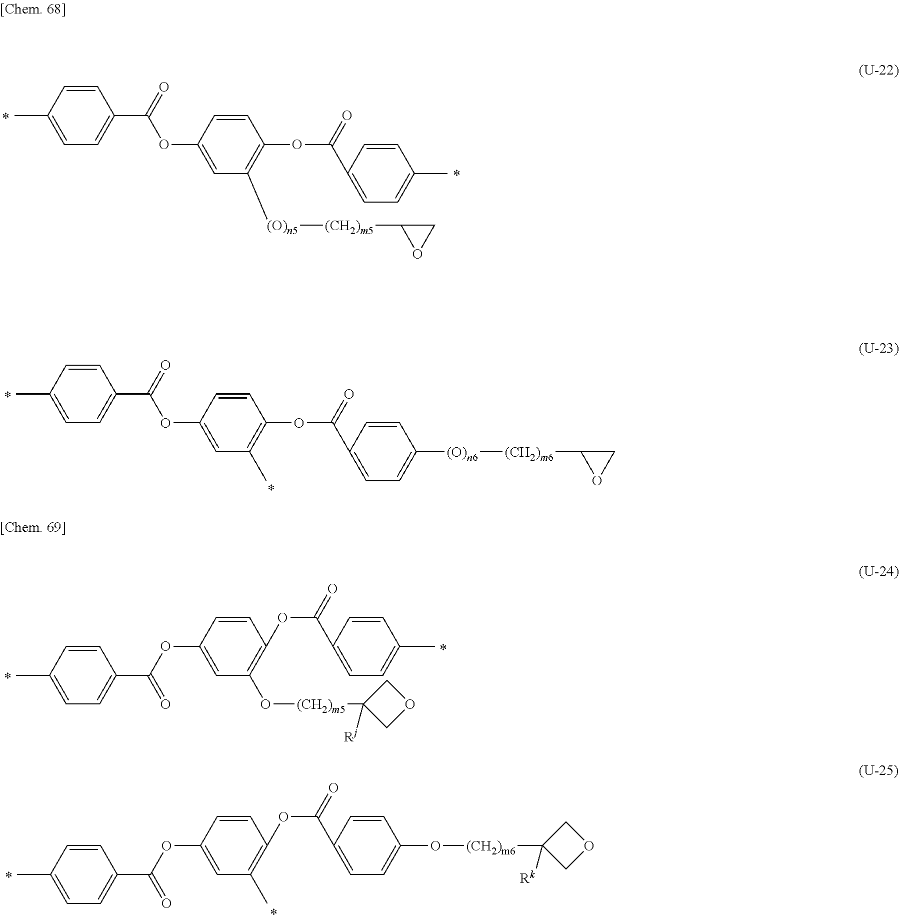

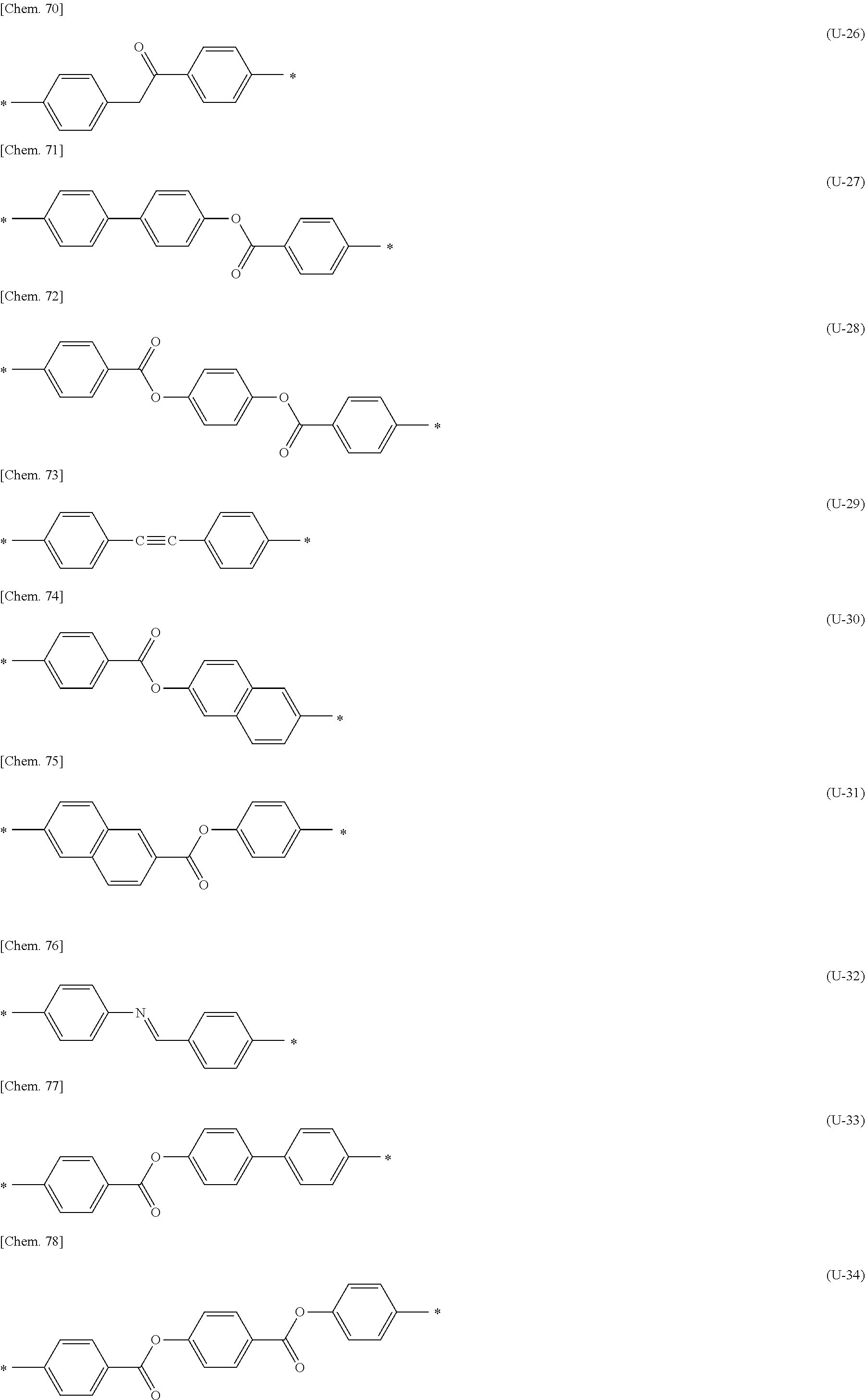

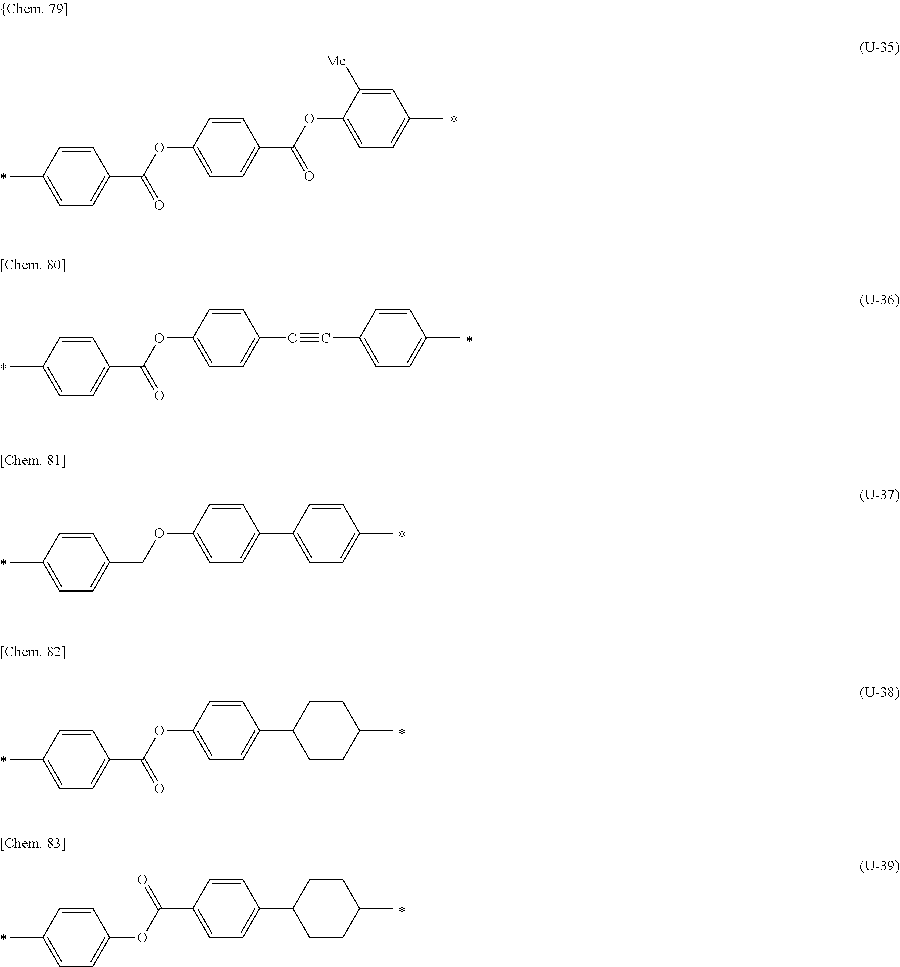

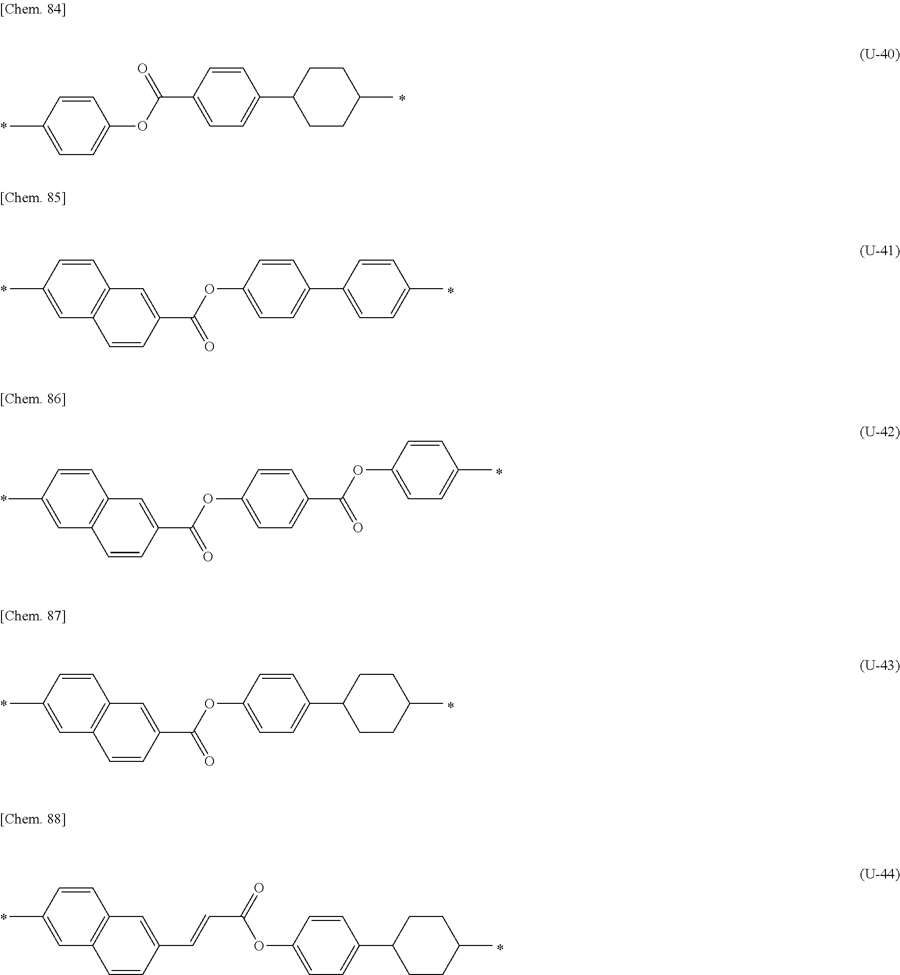

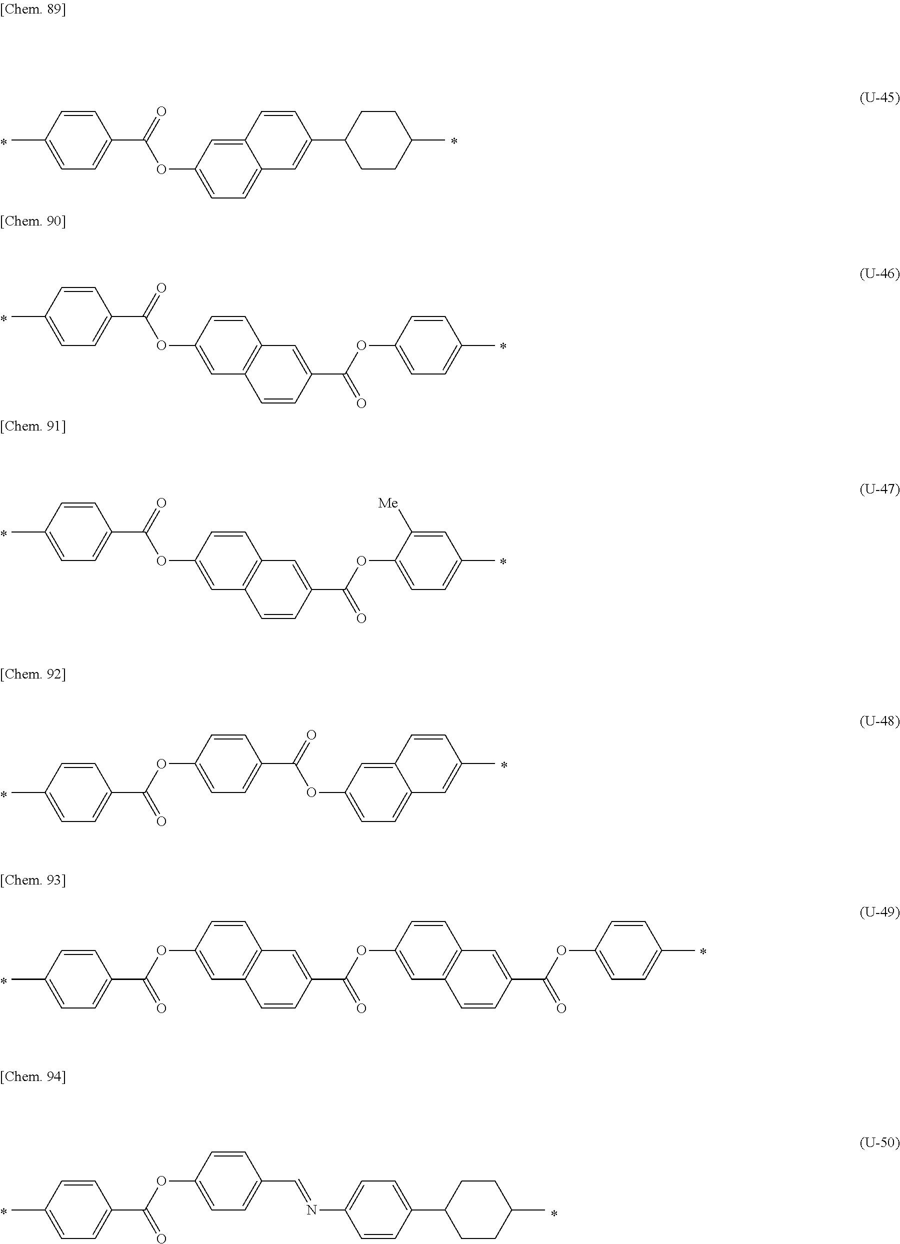

In general formula (i) according to the present invention, SPⁱ¹ may be a divalent organic group optionally with hydrogen atom(s) therein substituted with general formula (i-3) below.

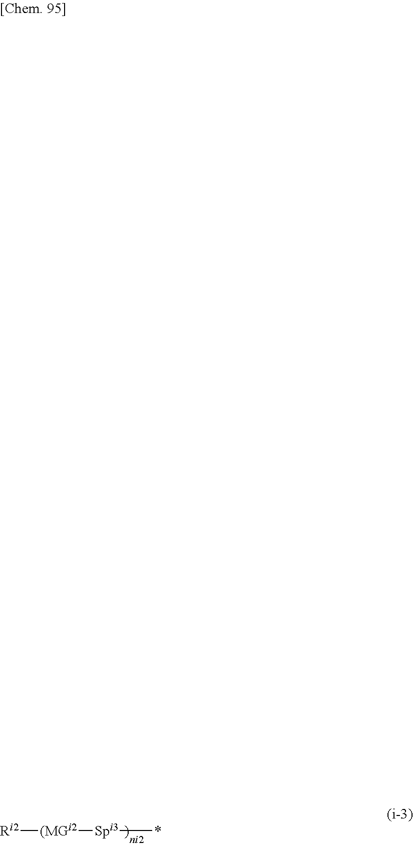

(In general formula (i-3) above, MGⁱ² represents a mesogenic group,

SPⁱ³ represents a single bond or spacer group,

Rⁱ² represents a hydrogen atom, halogen atom, cyano group, or C1-18 linear or branched alkyl group, where one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—, and the hydrogen atom, halogen atom, cyano group, or one or more hydrogen atoms in the alkyl group may be substituted with general formula (i-4)

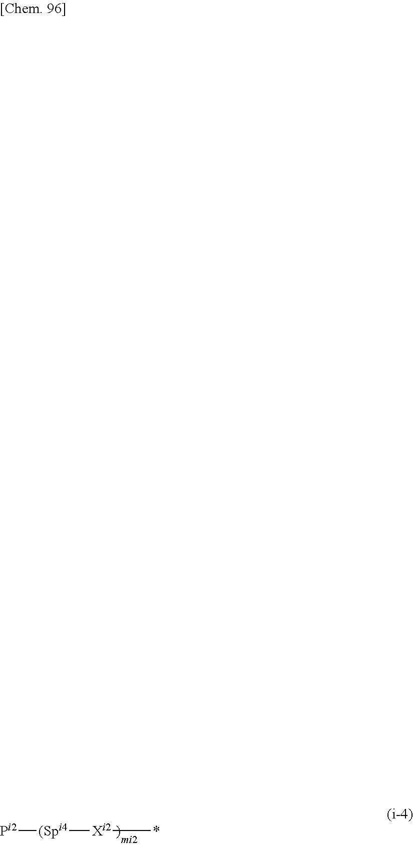

(In general formula (i-4) above, Pⁱ² represents a reactive functional group,

Spⁱ⁴ represents a single bond or C1-18 alkylene group, where the hydrogen atoms in the alkylene group may be substituted with one or more halogen atoms or CNs, and one CH₂ group present in the alkylene group, or each of nonadjacent two or more independently, may be substituted with —O—, —COO—, —OCO—, or —OCO—O—,

Xⁱ² represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P-Spⁱ⁴ and Spⁱ⁴-X, however, include no —O—O—, —O—NH—, —S—S—, or —O—S— group.), ni2 represents an integer of 0 to 8, mi2 represents 0 or 1, * represents a bond, and if ni2 is 2 or more and there are multiple MGⁱ²s or SPⁱ³s, they may be the same or different.),

MGⁱ² in general formula (i-3) above represents a mesogenic group, and preferred forms of MGⁱ²(s) are the same as those for MGⁱ¹ in general formula (i) and are not described. If SPⁱ¹ in general formula (i) is substituted with general formula (i-3) above, MGⁱ¹ and MGⁱ² may be the same or different.

SPⁱ³ in general formula (i-3) above represents a single bond or spacer group, and preferred forms of SPⁱ³ are the same as those for SPⁱ¹ in general formula (i) and are not described. If SPⁱ¹ in general formula (i) is substituted with general formula (i-3) above, SPⁱ¹ and SPⁱ³ may be the same or different.

Preferred forms of Rⁱ² in general formula (i-3) above are the same as those for Rⁱ¹ in general formula (i) and are not described. If SPⁱ¹ in general formula (i) is substituted with general formula (i-3) above, Rⁱ¹ and Rⁱ² may be the same or different.

Preferred forms of ni2 in general formula (i-3) above are the same as those for ni1 in general formula (i) and are not described.

Preferred forms of Pⁱ² in general formula (i-4) above are the same as those for Pⁱ¹ in general formula (i-1) and are not described.

Preferred forms of SPⁱ⁴ in general formula (i-4) above are the same as those for Spⁱ² in general formula (i-1) and are not described.

Preferred forms of Xⁱ² in general formula (i-4) above are the same as those for Xⁱ¹ in general formula (i-1) and are not described.

In general formula (i) according to the present invention, MGⁱ¹ may be a divalent organic group incorporating a cyclic group optionally with any hydrogen atom(s) in the cyclic group substituted with general formula (i-3) above.

If MGⁱ¹ in general formula (i) is substituted with general formula (i-3) above, MGⁱ¹ and MGⁱ² may be the same or different, if MGⁱ¹ in general formula (i) is substituted with general formula (i-3) above, SPⁱ¹ and SPⁱ³ may be the same or different, and if MGⁱ¹ in general formula (i) is substituted with general formula (i-3) above, Rⁱ¹ and Rⁱ² may be the same or different. Preferred forms of general formulae (i-3) and (i-4) in the case in which MGⁱ¹ in general formula (i) is substituted with general formula (i-3) above are the same as in the case in which Spⁱ¹ in general formula (i) is substituted with general formula (i-3) above.

General formula (i) according to the present invention is preferably one or two or more selected from the group consisting of the compounds represented by general formulae (A-1) to (A-10) and (B-1) to (B-7) below, preferably one or two or more selected from the group consisting of the compounds represented by general formulae (A-1-1) to (A-10-1) and of general formulae (B-1-1) to (B-7-1).

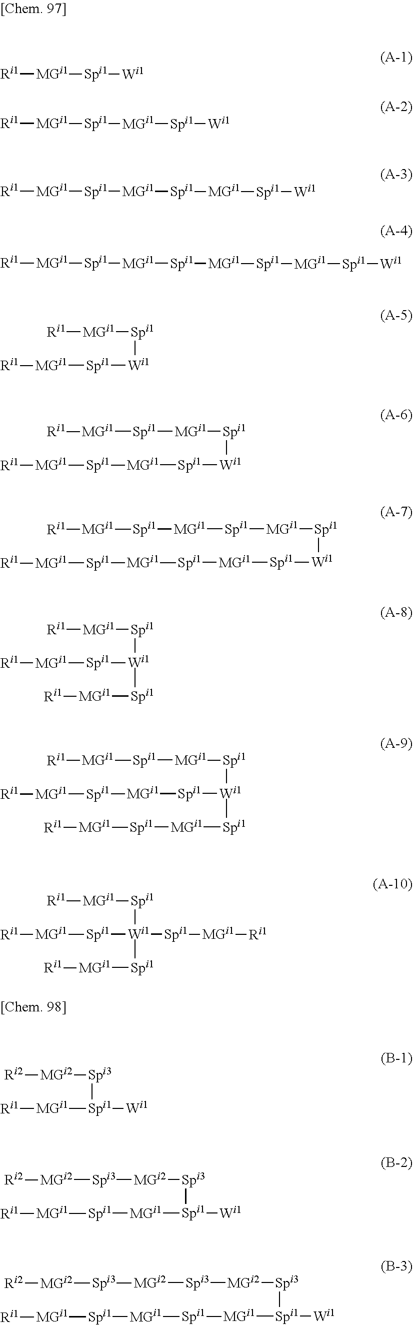

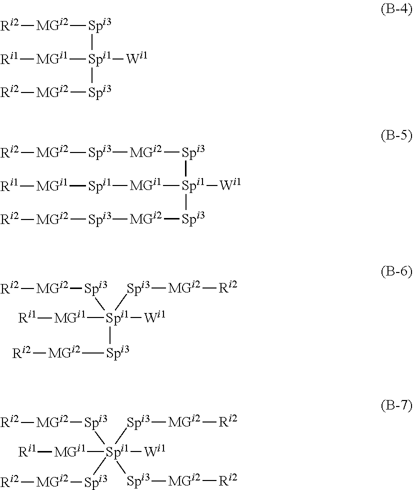

Rⁱ¹, Rⁱ², MGⁱ¹, MGⁱ², Wⁱ¹, Spⁱ¹, and Spⁱ² in the compounds represented by general formulae (A-1) to (A-10) and (B-1) to (B-7) above have the same meaning as in general formulae (i), (i-1), (i-3), etc., above.

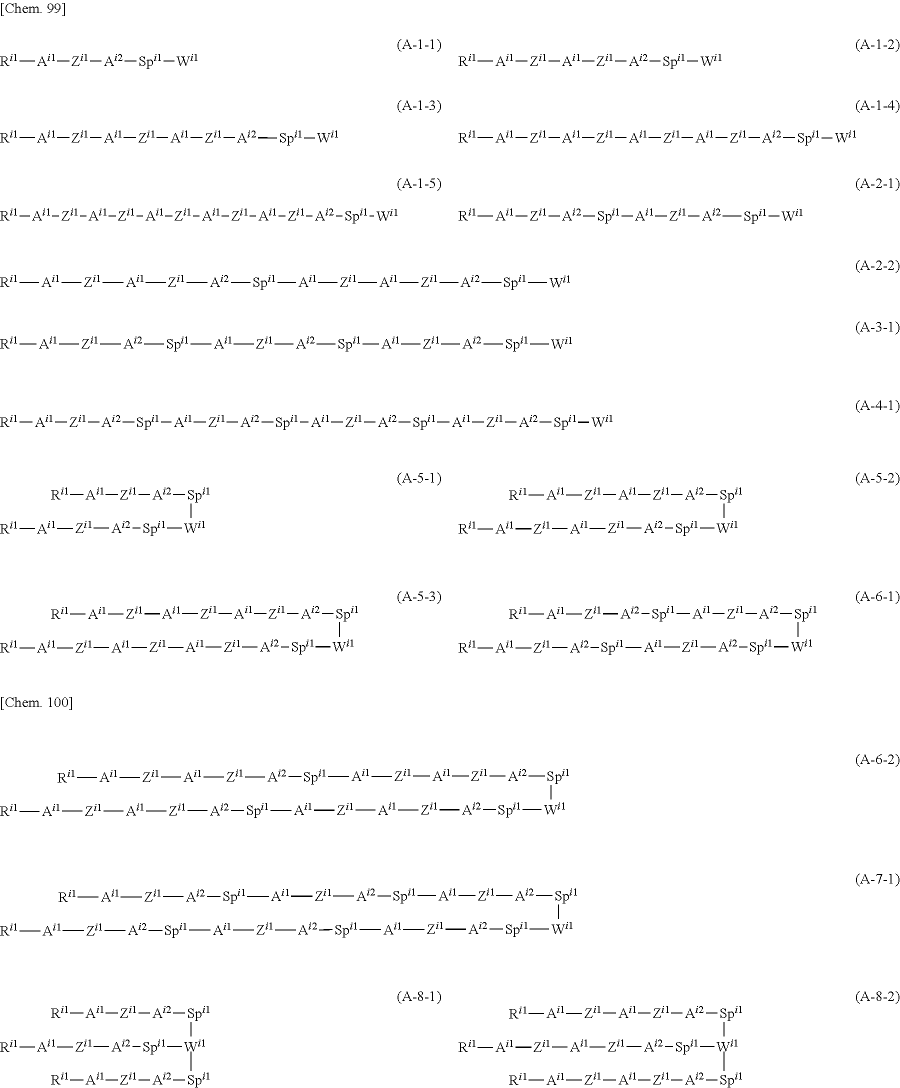

Rⁱ¹, Rⁱ², Aⁱ¹, Aⁱ², Zⁱ¹, Wⁱ¹, Spⁱ¹, and Spⁱ² in the compounds represented by general formulae (A-1-1) to (A-8-2) above have the same meaning as in general formulae (i), (i-1), (i-3), (i-5), etc., above.

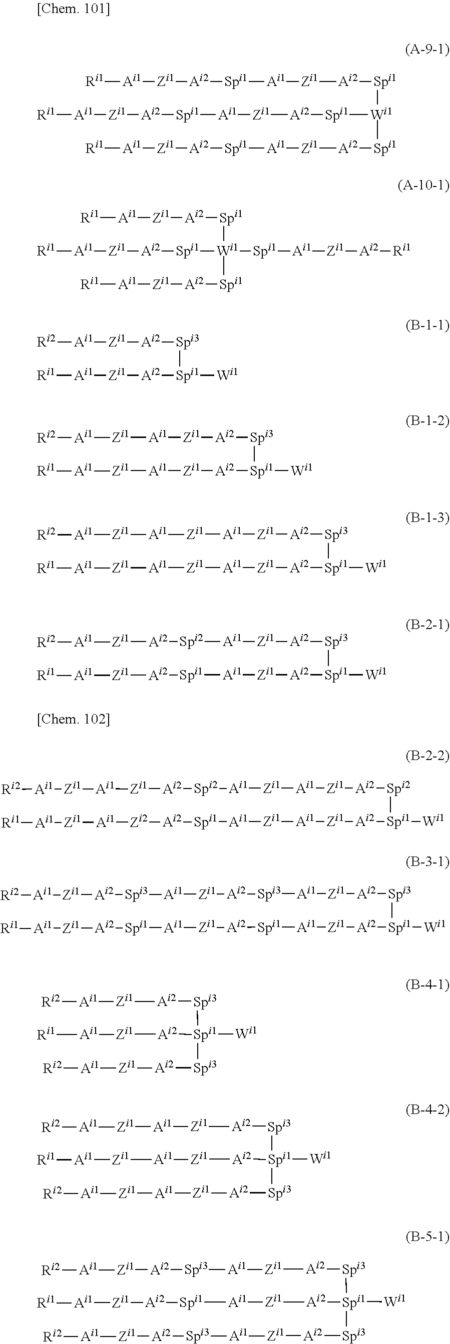

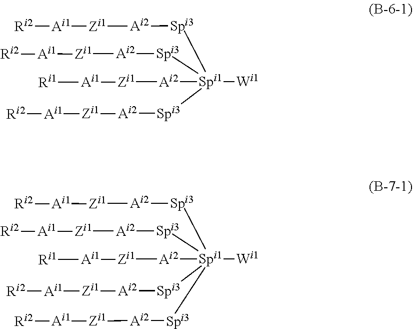

Rⁱ¹, Rⁱ², Aⁱ¹, Aⁱ², Zⁱ¹, Wⁱ¹, Spⁱ¹, and Spⁱ² in the compounds represented by general formulae (A-9-1) and (A-10-1) above and in general formulae (B-1-1) to (B-7-1) above have the same meaning as in general formulae (i), (i-1), (i-3), (i-5) etc., above.

Specifically, particularly preferred surface-modifying compounds according to the present invention are the compounds listed in formulae (1-1) to (1-124) below.

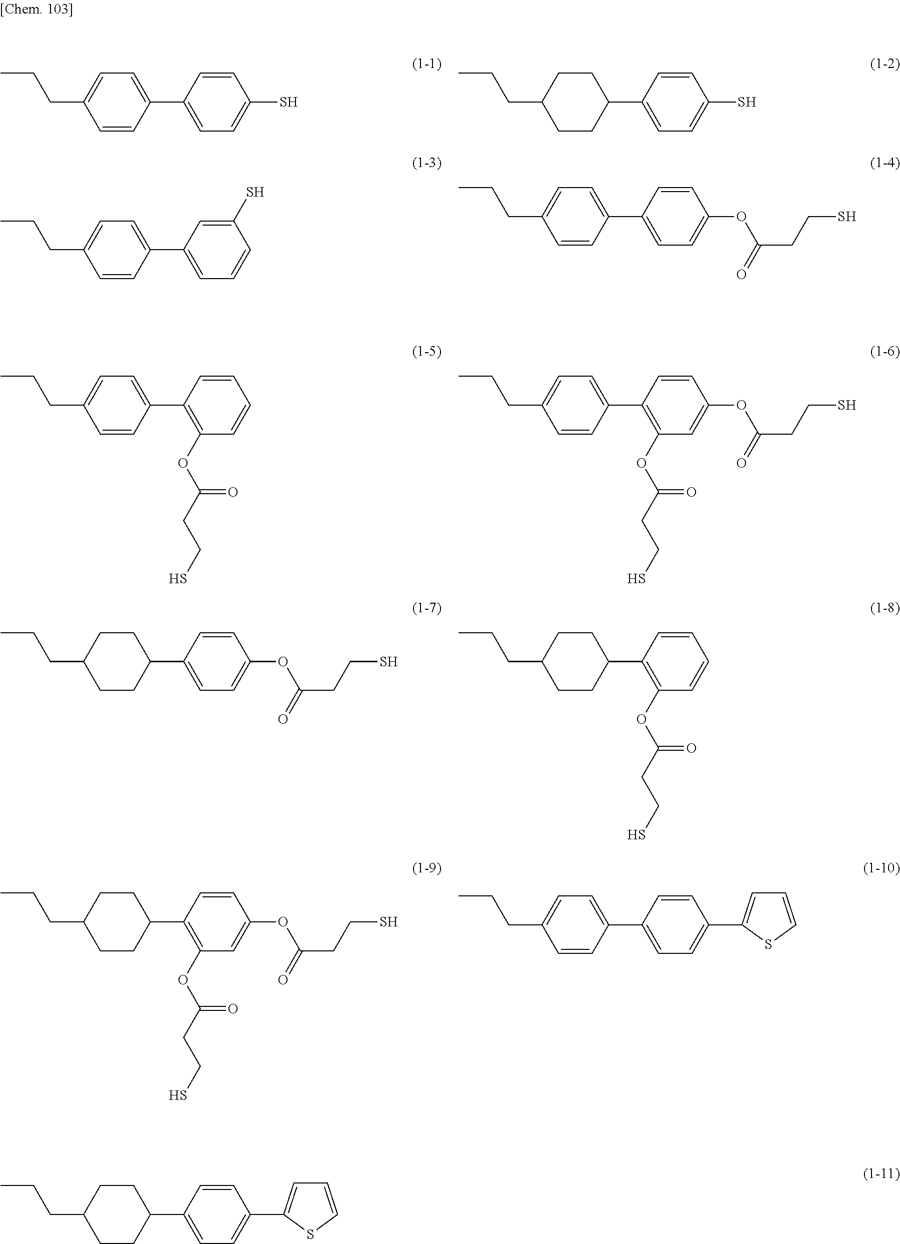

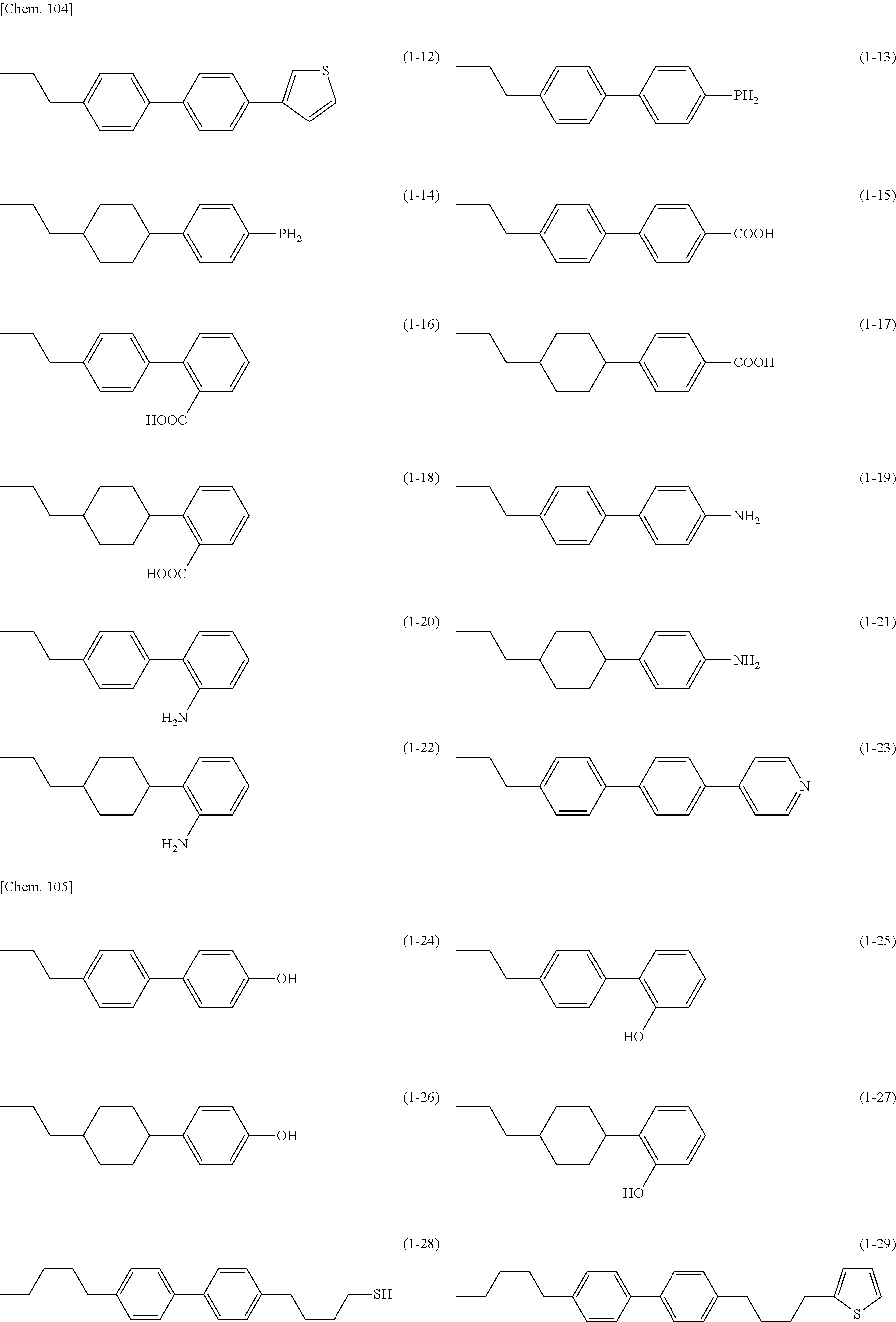

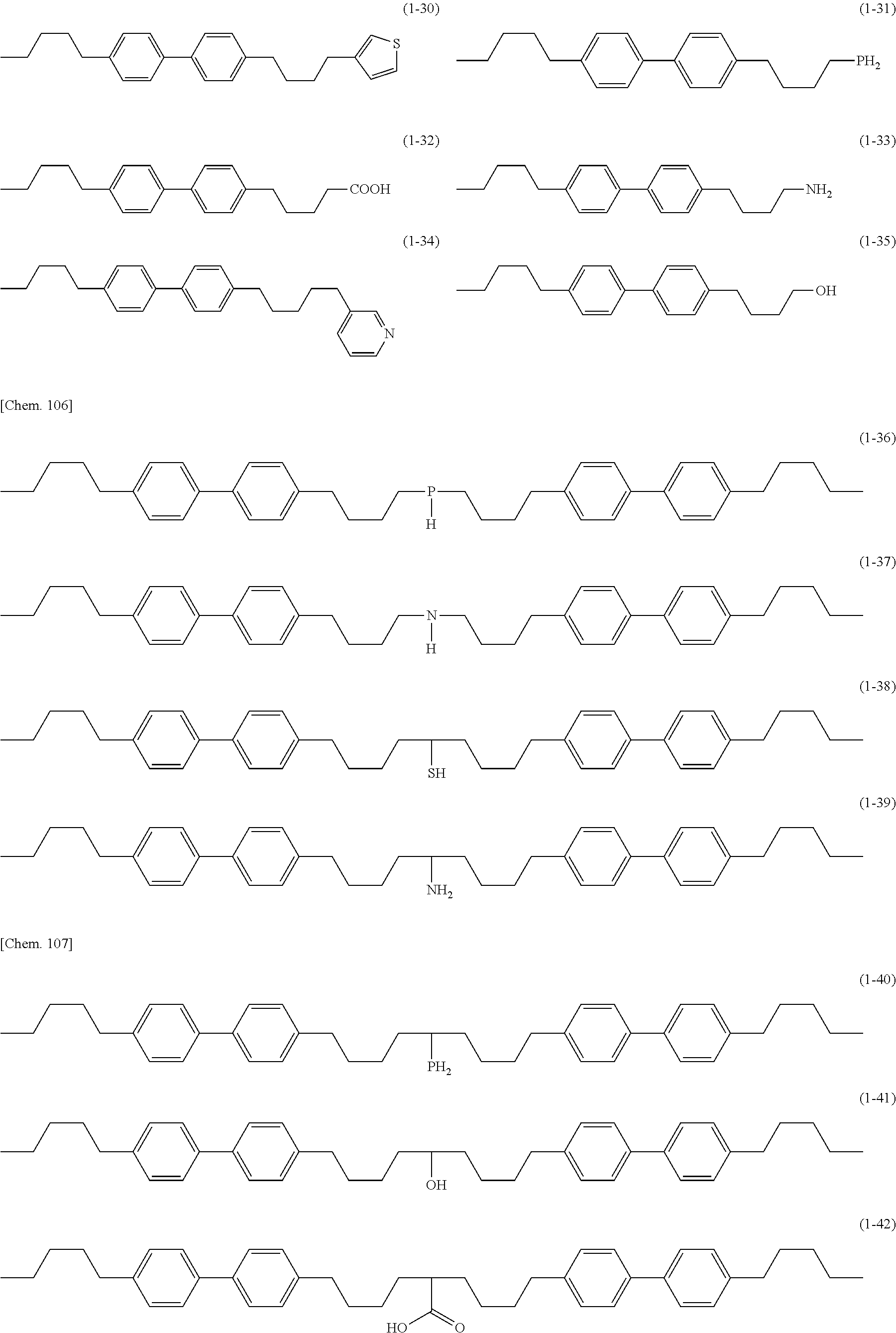

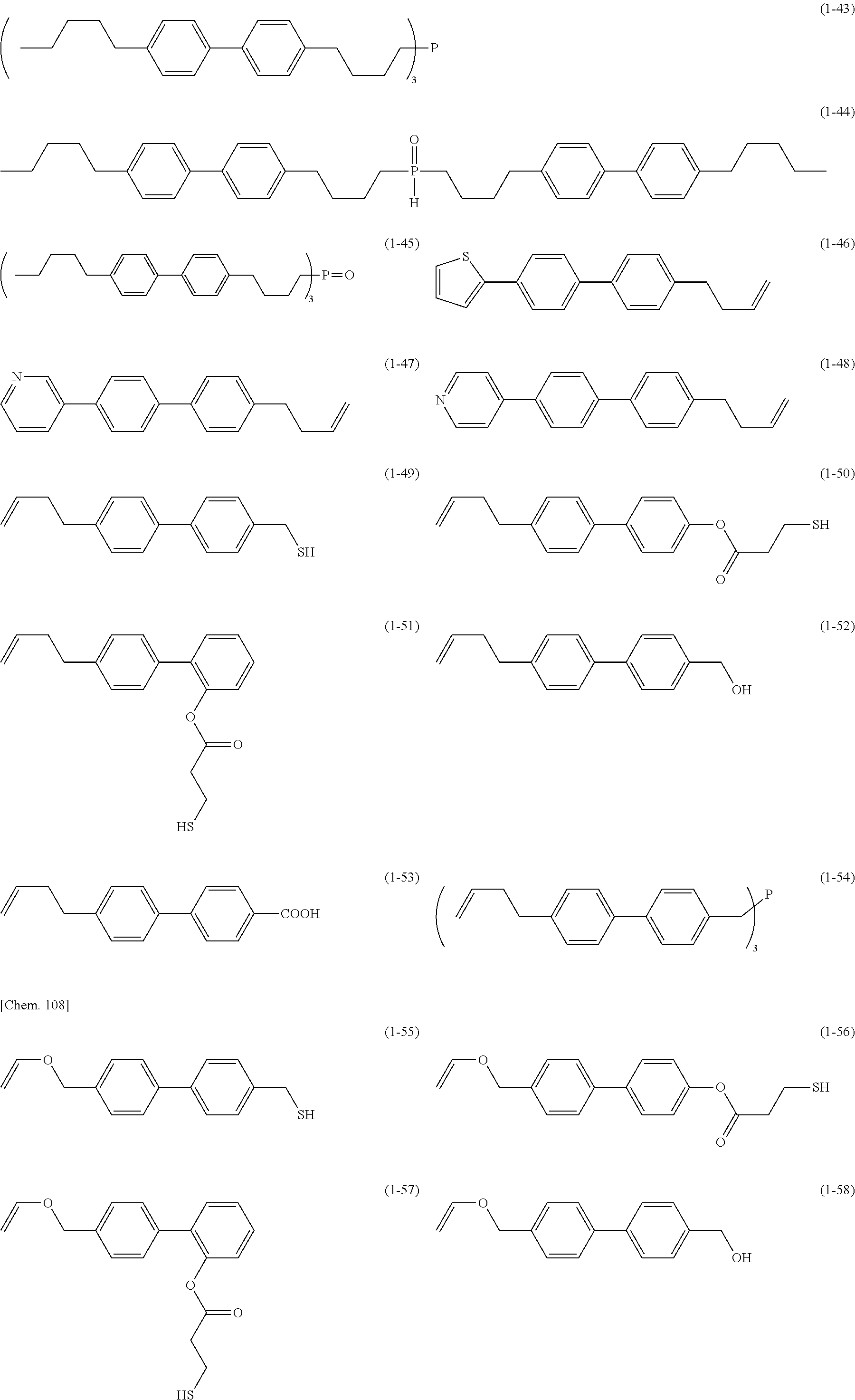

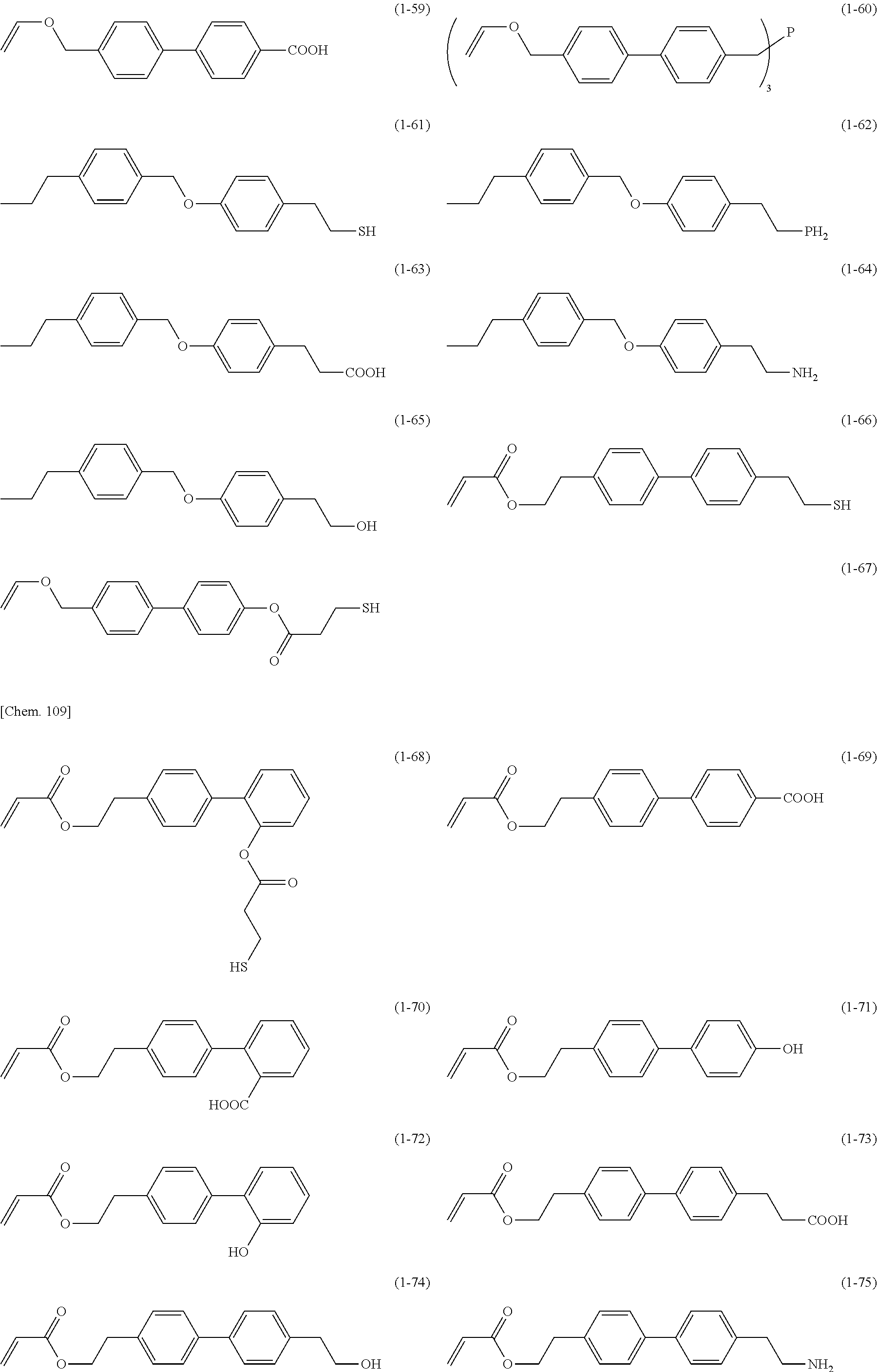

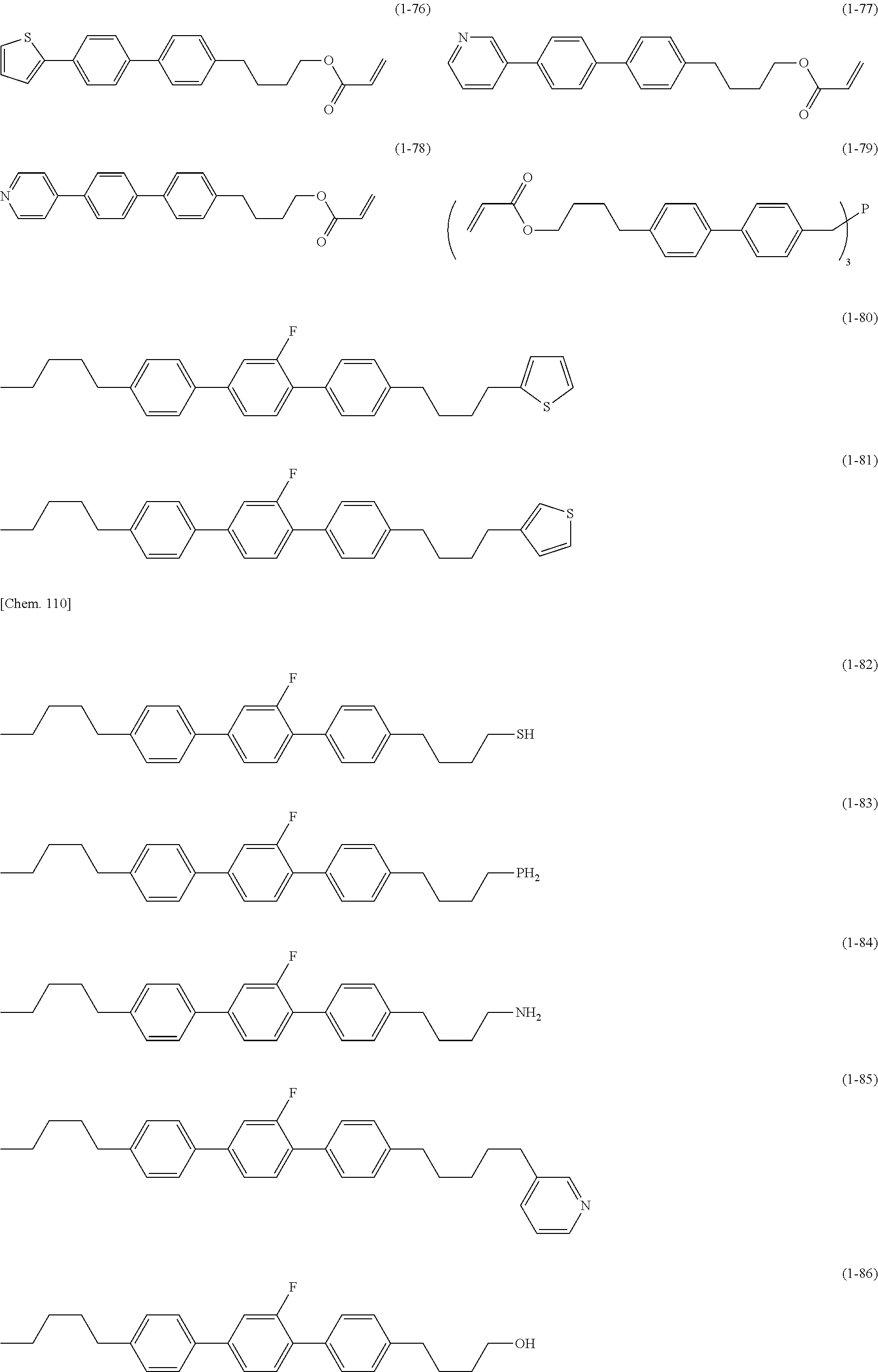

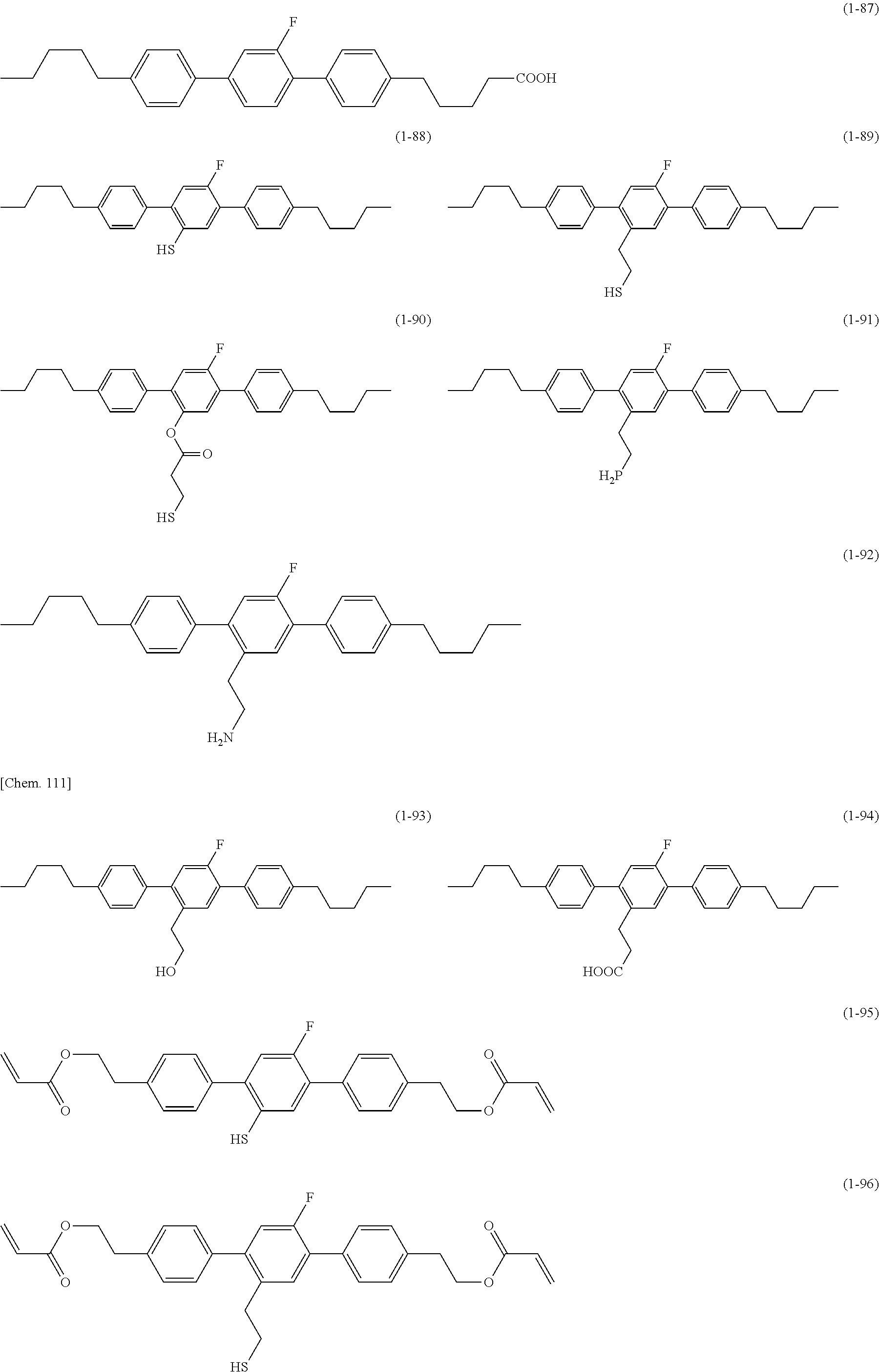

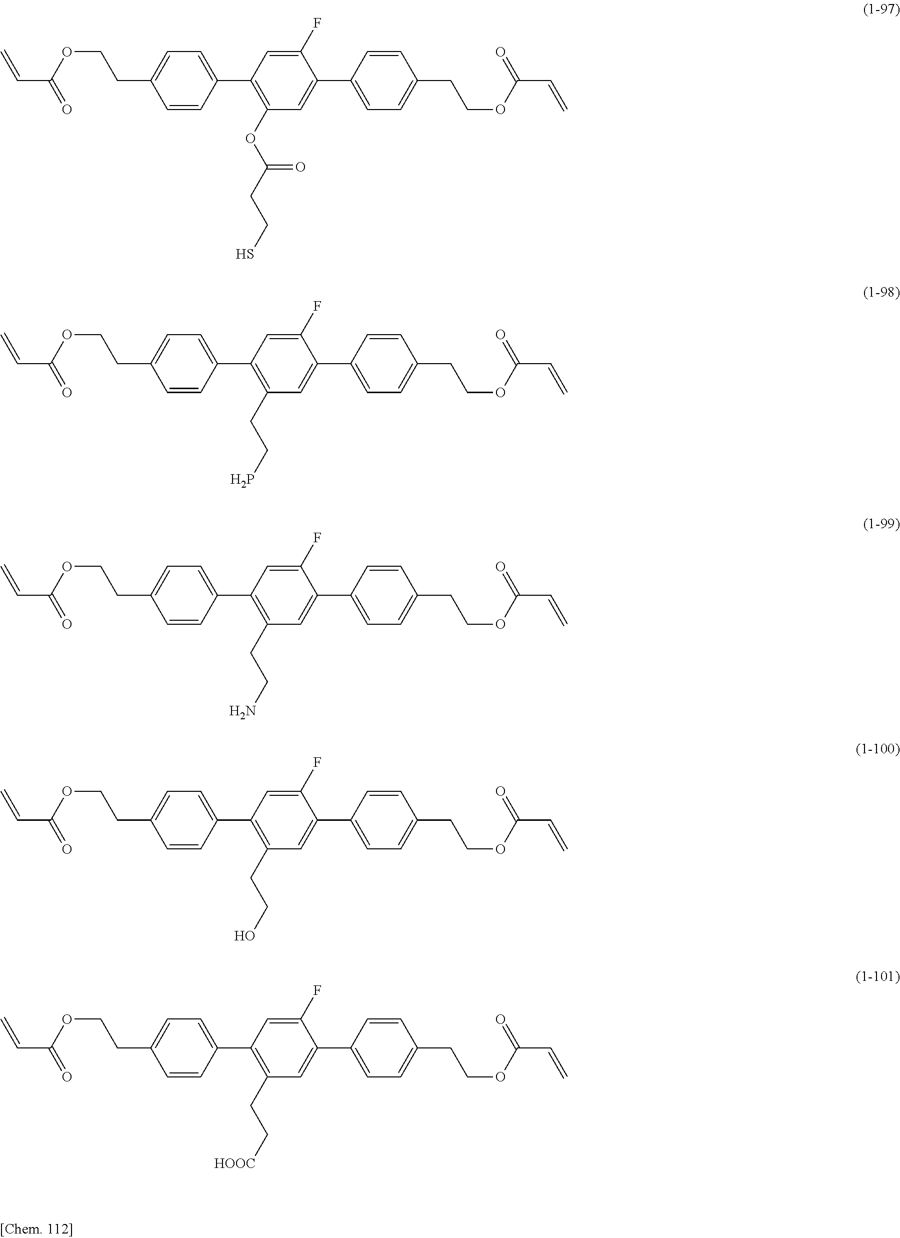

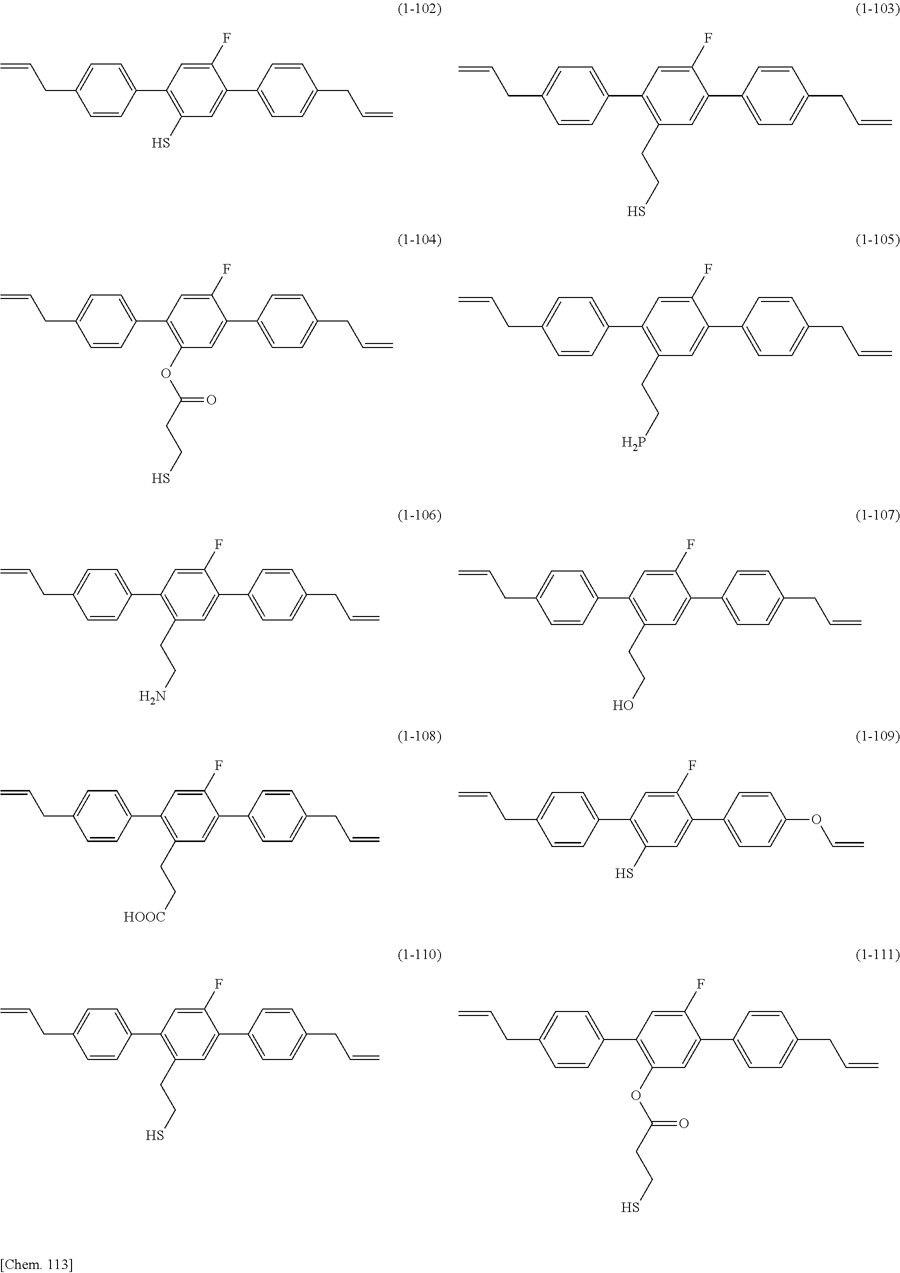

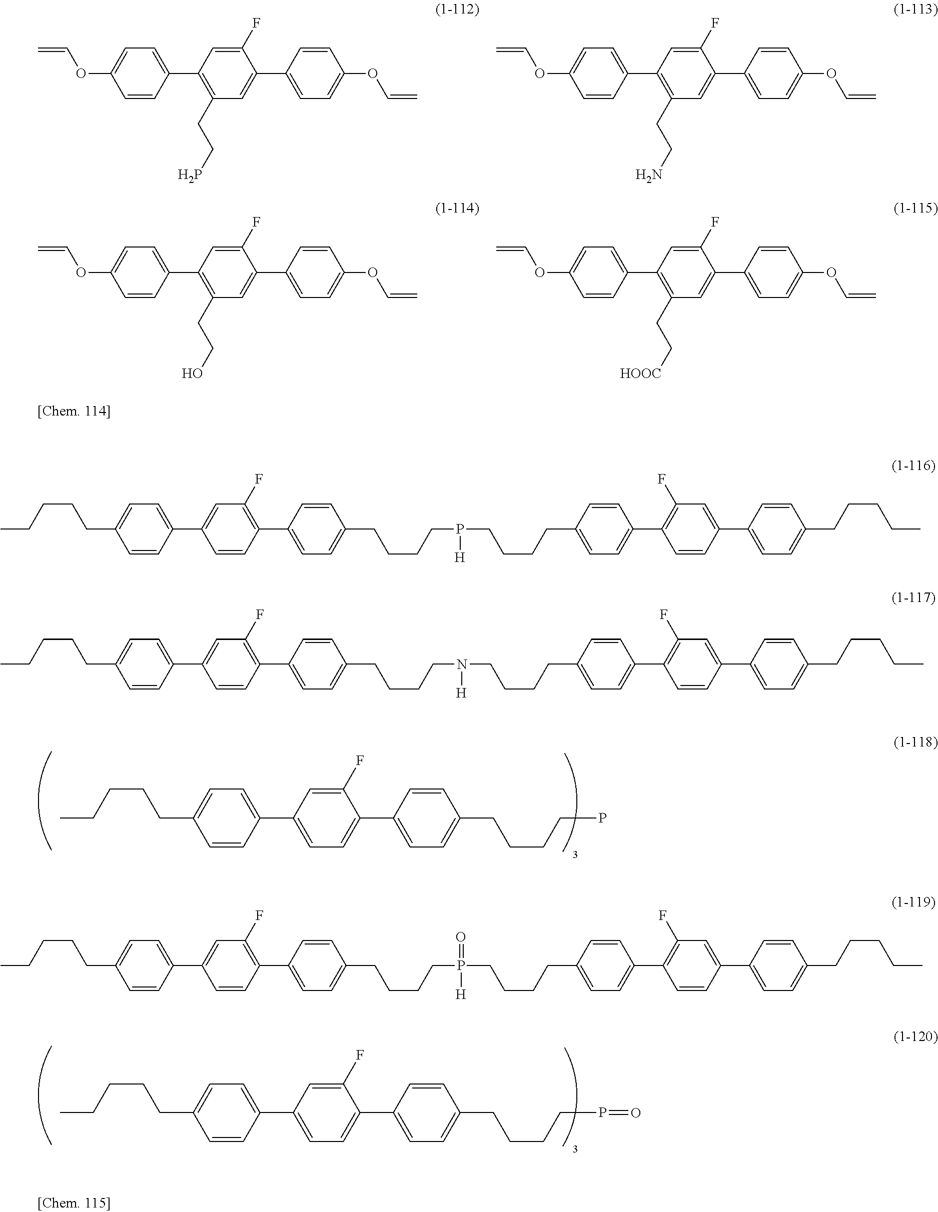

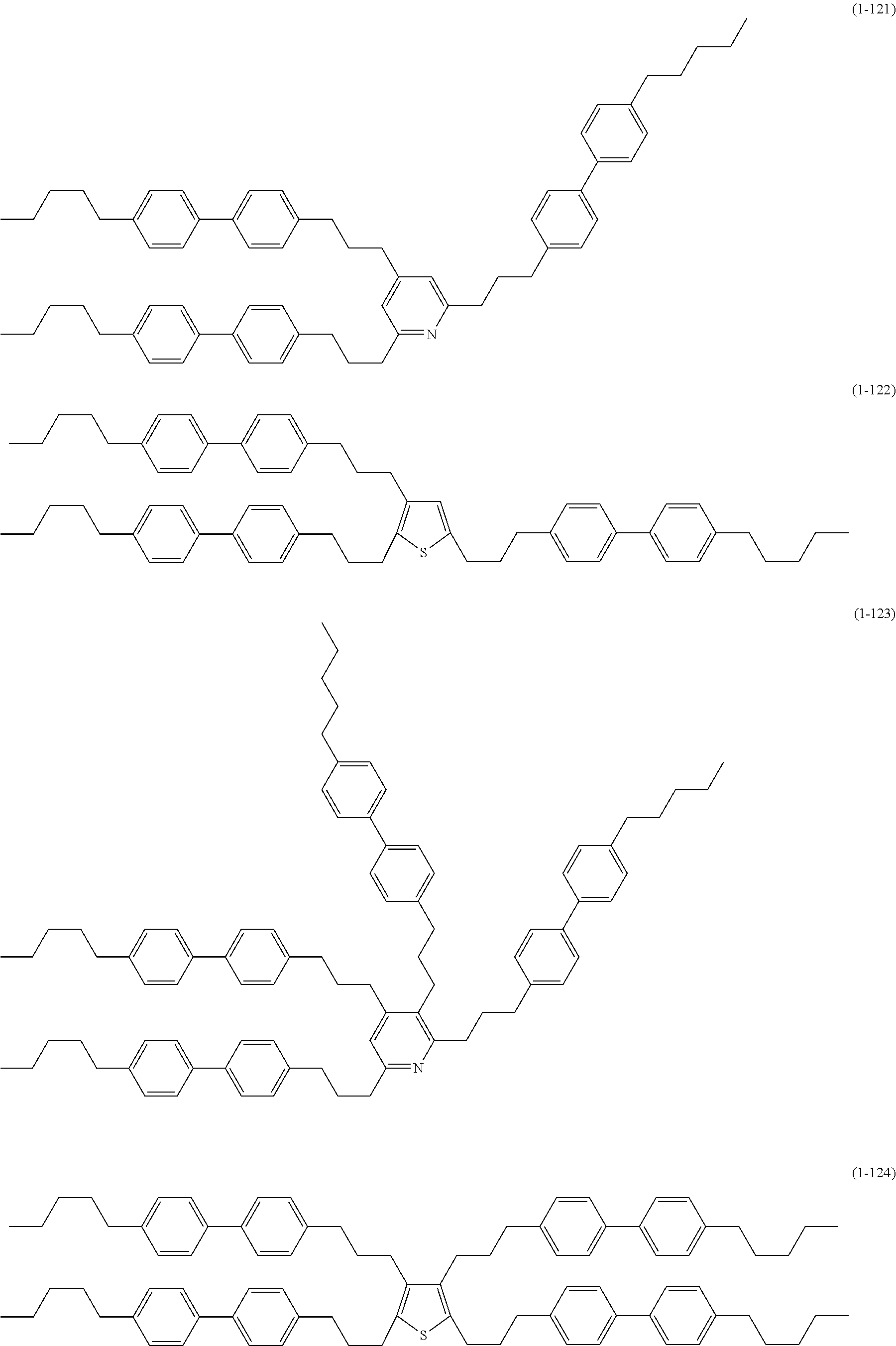

A second of the present invention is the compounds represented by general formula (i) above.

The compounds represented by general formula (i) above are ligands that modify the surface of luminescent nanocrystals, such as quantum dots or quantum rods. These compounds not only provide the function of protecting the luminescent nanocrystals from aggregation therebetween and from their surrounding chemical environment, but also help disperse the nanocrystals orderly over a broad temperature range.

If so-called quantum dots are used as luminescent nanocrystals according to the present invention, they may be synthesized by a method in which aggregated micelles are used, a known hot-soap method, or a known production method described in, for example, Japanese Unexamined Patent Application Publication (Translation of PCT Application) No. 2007-537886, 2010-532409, 2012-507588, or 2011-530187, or commercially available quantum dots may be used. If so-called quantum rods are used as luminescent nanocrystals according to the present invention, they may be synthesized by the known production method described in Nature Vol, 404, 59-61, or commercially available quantum rods may be used.

Moreover, they may be synthesized as in Method for Synthesizing the Synthesis of CdSe Quantum Rods (1) or Method for Synthesizing CdSe Quantum Rods (2) below.

“Method for Synthesizing the Synthesis of CdSe Quantum Rods (1)”

A flask was charged with 0.048 g (0.375 mmol) of CdO, 11.33 g (28.4 mmol) of TOPO, and 0.67 g (2.4 mmol) of TDPA (tetradecylphosphonic acid), and the inside of the flask was evacuated. The mixture was then heated to 60° C. to melt TOPO, and the solution was stirred with a stirrer. Subsequently, the solution temperature was raised to 300° C. and held for 2 hours so that CdO would be decomposed sufficiently. The solution temperature was then lowered to 270° C., and 0.485 ml of 1 M TOP-Se (solution of Se in TOP) was injected thereto quickly. Immediately after that, the solution temperature was lowered to 250° C. and held for 30 minutes to allow crystals to grow into quantum rods. To this reaction solution, methanol was added to cause the particles to aggregate, and the particles were precipitated using a centrifuge. The supernatant was discarded. This operation was repeated twice, and finally the particles were dispersed in toluene. The aspect ratio of the resulting particles was between 4 and 5.

“Method for Synthesizing CdSe Quantum Rods (2)”

A stock solution was prepared by charging a flask with 0.82 g of Cd(CH₃)2, 1.6 g of 20 wt % TBP-Se (20 wt % solution of Se in tributylphosphine), and 14.08 g of TBP (Cd/Se=1.4/1 (molar ratio)), stirring the mixture for 5 minutes, and then cooling the solution in a freezer at −20° C. Another flask was charged with 3.68 g of TOPO and 0.32 g of HPA (hexylphosphonic acid), and the mixture was heated to 360° C. Then the stock solution was removed from the freezer and agitated vigorously for 10 seconds. Using a syringe, 2.0 ml of this solution was injected to a TOPO/HPA mixture quickly (approximately 0.1 seconds) in an Ar atmosphere. This lowered the solution temperature to about 300° C. The solution temperature was held at 300° C. for 30 minutes to obtain quantum rods. To this reaction solution, methanol was added to cause the particles to aggregate, and the particles were precipitated using a centrifuge. The supernatant was discarded. This operation was repeated twice, and finally the particles were dispersed in toluene. The aspect ratio of the resulting particles was 5.

A luminescent nanocrystal complex according to the present invention can be produced by any method, but examples include ligand exchange and coordination during the synthesis of the luminescent nanocrystals, which are described in detail in, for instance, the Examples set forth hereinafter. The ligand exchange in the present invention is a method in which a ligand present on the surface of quantum dots or luminescent nanocrystals is exchanged for another that has a functional group with a higher coordination potential. Ligands have a substituent that contains an atom, such as sulfur, phosphorus, nitrogen, or oxygen, for example at an end of a linear alkyl chain, and examples include ligands such as thioether, thiol, phosphine, phosphine oxide, amine, a hydroxyl group, and carboxylic acid. These ligands vary in their tendency to become adsorbed onto the surface of nanoparticles in accordance with the kind of substituent. Ligand A coordinated to the surface of the nanoparticles is replaced in a solution with ligand B that has a stronger tendency to become adsorbed. For example, if the initial surface-modifying ligand is the amino group, it can be exchanged for the thiol group or a similar ligand. The coordination potential is generally said to become stronger in accordance with the atom borne by the substituent in the order of sulfur>phosphorus>nitrogen>oxygen.

A third of the present invention is a composition that contains a luminescent nanocrystal complex and a binder component.

As the binder component according to the present invention, materials such as a monomer for binder applications, a binder resin, a liquid crystal polymer, a liquid-crystalline monomer having a polymerizable functional group, and a polymer of a liquid-crystalline monomer having a polymerizable functional group are preferred.

The binder component according to the present invention preferably has a mesogenic backbone. Since the surface-modifying compound in a luminescent nanocrystal complex according to the present invention has a mesogenic group, the mesogenic backbone improves the compatibility with this structurally mesogenic ligand, thereby improving dispersibility.

As the monomer for binder applications, any monomer used to synthesize a known monomer is preferred and preferably has a mesogenic group. Examples include epoxy acrylate, epoxy, urethane, phenol, urea-melamine, polyester, polyolefin, polystyrene, polycarbonate, (meth)acrylic, silicone, polyvinyl chloride, and polyvinylidene chloride monomers.

The binder resin is preferably a resin that does not reduce the luminescence intensity of the luminescent nanocrystals. Examples include epoxy acrylate, epoxy, urethane, phenolic, urea-melamine, polyester, polyolefin, polystyrene, polycarbonate, (meth)acrylic, silicone, polyvinyl chloride, and polyvinylidene chloride resins.

For a membrane or film made by curing the composition according to the present invention to be drawn, polyester resins are preferred because of their superior mechanical strength. Polyethylene terephthalate and polyethylene naphthalate are more preferred.

The binder resin according to the present invention, moreover, preferably has a mesogenic backbone.

The surface-modifying compound in a luminescent nanocrystal complex according to the present invention has a mesogenic group. To increase the compatibility with this structurally mesogenic ligand and thereby improve dispersibility, it is preferred that the binder component or binder resin is structurally mesogenic. For example, a liquid crystal polymer or a polymer of a liquid-crystalline monomer having a polymerizable functional group is preferred.

The luminescent nanocrystal complex according to the present invention is preferably bound covalently to the binder component. More preferably, the luminescent nanocrystal complex according to the present invention is bound covalently to a binder resin.

The liquid crystal polymer according to the present invention is preferably a polymer liquid crystal whose backbone has a mesogenic group. Examples include polyester, polyamide, polycarbonate, polyimide, polyurethane, polybenzimidazole, polybenzoxazole, polyazomethine, polyesteramide, polyester carbonate, and polyester imide polymers and compositions thereof.

The liquid-crystalline monomer according to the present invention having a polymerizable functional group can be of any type. For example, a compound represented by general formula (II) below is preferred.

[Chem. 116]

P²¹-(Sp²¹-X²¹)_{q21}-MG-R²¹ (II)

In general formula (II) above, P²¹ represents a polymerizable functional group,

in general formula (II) above, Sp²¹ represents a C1-18 alkylene group (The hydrogen atoms in the alkylene group may be substituted with one or more halogen atoms, CN groups, or groups having a polymerizable functional group, and one CH₂ group present in this alkylene group, or each of nonadjacent two or more independently, may be substituted with —O—, —COO—, —OCO—, or —OCO—O—.),

in general formula (II) above, X²¹ represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P²¹—Sp²¹ and Sp²¹-X²¹, however, include no —O—O—, —O—NH—, —S—S—, or —O—S— group.),

in general formula (II) above, q21 represents 0 or 1,

in general formula (II) above, MG represents a mesogenic group,

in general formula (II) above, R²¹ represents a hydrogen atom, halogen atom, cyano group, or C1-12 linear or branched alkyl group, where the alkyl group may be linear or branched, and one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, or alternatively R²¹ represents general formula (II-a),

[Chem. 117]

*X²²-Sp²²)_{q22}-P²² (II-a)

(In general formula (II-a) above, P²² represents a polymerizable functional group, Sp²² represents the same as defined by Sp²¹, X²² represents the same as defined by X²¹ (P²²—Sp²² and Sp²²-X²², however, include no —O—O—, —O—NH—, —S—S—, or —O—S— group.), and q22 represents 0 or 1.)

the mesogenic group, represented by MG, is represented by general formula (II-b)

[Chem. 118]

*B1-Z1)ᵣ₁-B2-Z2-B3-* (II-b)

(In general formula (II-b) above, B1, B2, and B3 each independently represent a 1,4-phenylene, 1,4-cyclohexylene, 1,4-cyclohexenyl, tetrahydropyran-2,5-diyl, 1,3-dioxan-2,5-diyl, tetrahydrothiopyran-2,5-diyl, 1,4-bicyclo(2,2,2)octylene, decahydronaphthalen-2,6-diyl, pyridin-2,5-diyl, pyrimidin-2,5-diyl, pyrazin-2,5-diyl, thiophen-2,5-diyl-, 1,2,3,4-tetrahydronaphthalen-2,6-diyl, 2,6-naphthylene, phenanthren-2,7-diyl, 9,10-dihydrophenanthren-2,7-diyl, 1,2,3,4,4a,9,10a-octahydrophenanthren-2,7-diyl, 1,4-naphthylene, benzo[1,2-b:4,5-b′]dithiophen-2,6-diyl, benzo[1,2-b:4,5-b′]diselenophen-2,6-diyl, [1]benzothieno[3,2-b]thiophen-2,7-diyl, [1]benzoselenopheno[3,2-b]selenophen-2,7-diyl, or fluoren-2,7-diyl group, optionally having, as substituent(s), one or more F, C1, CF₃, OCF₃, CN groups, C1-8 alkyl groups (The hydrogen atoms in the alkyl group(s) may be substituted with one or more phenyl groups, and one CH₂ group present in this group(s), or each of nonadjacent two or more independently, may be substituted with —O—, —COO—, —OCO—, or —OCO—O—.), C1-8 alkoxy groups, C1-8 alkanoyl groups, C1-8 alkanoyloxy groups, C1-8 alkoxycarbonyl groups, C2-8 alkenyl groups, C2-8 alkenyloxy groups, C2-8 alkenoyl groups, or C2-8 alkenoyloxy groups, and/or general formula (II-c)

[Chem. 119]

*X²³)_{q24}-(Sp²³)_{q23}-P²³ (II-C)

(In formula (II-c) above, P²³ represents a polymerizable functional group,

Sp²³ represents the same as defined by Sp²¹ above,

X²³ represents —O—, —COO—, —OCO—, —OCH₂—, —CH₂O—, —CH₂CH₂OCO—, —COOCH₂CH₂—, —OCOCH₂CH₂—, or a single bond, q23 represents 0 or 1, and q24 represents 0 or 1. (P²³—Sp²³ and Sp²³-X²³, however, include no —O—O—, —O—NH—, —S—S—, or —O—S— group.)), and

in general formula (II-b) above, Z1 and Z2 each independently represent —COO—, —OCO—, —CH₂CH₂—, —OCH₂—, —CH₂O—, —CH═CH—, —C≡C—, —CH═CHCOO—, —OCOCH═CH—, —CH₂CH₂COO—, —CH₂CH₂OCO—, —COOCH₂CH₂—, —OCOCH₂CH₂—, —C═N—, —N═C—, —CONH—, —NHCO—, —C(CF₃)₂—, a halogenated or non-halogenated C2-10 alkyl group, or a single bond, with the proviso that if Z1 and Z2 represent single bonds, adjacent two of the ring structures of B1, B2, and B3 above may form a cyclic group through the binding of their respective functional groups, r1 represents 0, 1, 2, or 3, and for B1 and Z1, multiple groups may be the same or different. * represents a bond.)

The polymerizable functional group(s) in the liquid-crystalline monomer having a polymerizable functional group is preferably epoxy acrylate, (meth)acrylate, or vinyl group(s). It is preferred to add a non-crystalline monomer to the liquid-crystalline monomer to improve, for example, adhesiveness and dispersibility.

The composition according to the present invention preferably contains a photopolymerization initiator. It is preferred that at least one or more photopolymerization initiators be contained. Specific examples include BASF's “Irgacure 651,” “Irgacure 184,” “Darocur 1173,” “Irgacure 907,” “Irgacure 127,” “Irgacure 369,” “Irgacure 379,” “Irgacure 819,” “Irgacure 2959,” “Irgacure 1800,” “Irgacure 250,” “Irgacure 754,” “Irgacure 784,” “Irgacure OXE01,” “Irgacure OXE02,” “Lucirin TPO,” “Darocur 1173,” and “Darocur MBF,” LAMBSON's “Esacure 1001M,” “Esacure KIP150,” “SpeedCure BEM,” “SpeedCure BMS,” “SpeedCure MBP,” “SpeedCure PBZ,” “SpeedCure ITX,” “SpeedCure DETX,” “SpeedCure EBD,” “SpeedCure MBB,” and “SpeedCure BP,” Nippon Kayaku's “Kayacure DMBI,” Nihon SiberHegner (currently DKSH)'s “TAZ-A,” ADEKA's “ADEKA Optomer SP-152,” “ADEKA Optomer SP-170,” “ADEKA Optomer N-1414,” “ADEKA Optomer N-1606,” “ADEKA Optomer N-1717,” and “ADEKA Optomer N-1919,” UCC's “Cyracure UVI-6990,” “Cyracure UVI-6974,” and “Cyracure UVI-6992,” Asahi Denka's “ADEKA Optomer SP-150, SP-152, SP-170, and SP-172,” Rhodia's “PHOTOINITIATOR 2074,” BASF's “Irgacure 250,” GE Silicones's “UV-9380C,” and Midori Kagaku's “DTS-102.”

As for the amount of photopolymerization initiator used, it is preferred to add 0.1 to 7 parts by mass, more preferably 0.5 to 6 parts by mass, even more preferably 1 to 6 parts by mass, in particular 3 to 6 parts by mass of it if the total amount of the composition is 100 parts by mass. One of these may be used alone, or two or more may be used as a mixture. A sensitizer, for example, may be added.

In the composition in the present invention, a known thermal polymerization initiator may be used in combination with a photopolymerization initiator. Given that the composition according to the present invention contains luminescent nanocrystals, photopolymerization initiators are preferred to thermal polymerization initiators.

The composition in the present invention may contain a binder component that has a repeating unit, for example formula (V-1-15) below.

The polymer (V-1-15) below

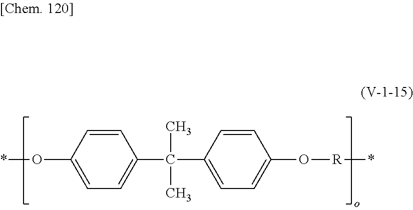

(where Rs each independently represent a C1-20 hydrocarbon group or aromatic hydrocarbon.)

In the present invention, an example is a polyimide and/or polyamide compound (V-3) having a repeating unit represented by, for example, the general formula below. The polyimide and/or polyamide compound (V-3) having a repeating unit only needs to have a repeating unit and may be a monomer, polymer, or a copolymer of the polyimide and/or polyamide compound and another compound having a polymerizable group, but preferably has a molecular weight Mw of 200000 or less and Mn of 400000 so that it is soluble in the solvent used in the composition. Specific examples of polyimide and/or polyamide compounds (V-3) include the polymers of formulae (V-3-1) to (V-3-4) below.

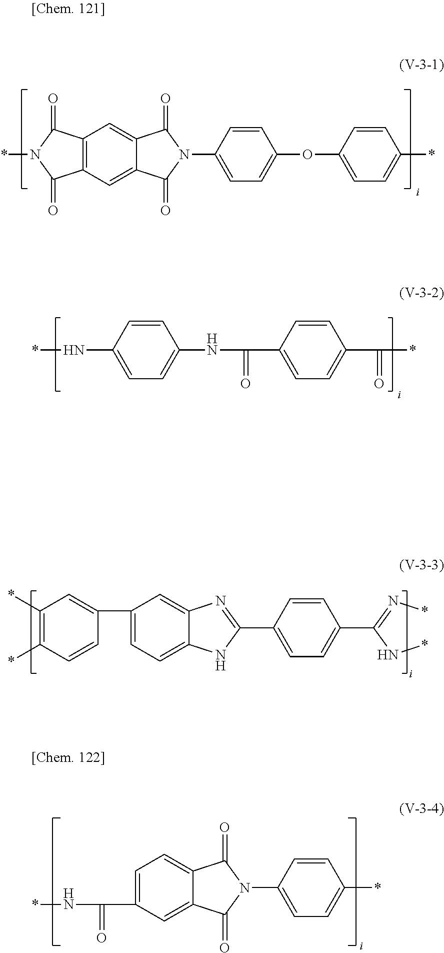

(where i represents an integer of 1 or more, but preferably is between 1 and 50.)

### (Organic Solvent)

An organic solvent may be added to the composition in the present invention. Any organic solvent can be used, but organic solvents in which polymerizable liquid-crystalline compounds are highly soluble are preferred, preferably organic solvents that can be dried at temperatures equal to or lower than 100° C. Examples of such solvents include aromatic hydrocarbons, such as toluene, xylene, cumene, and mesitylene, ester solvents, such as methyl acetate, ethyl acetate, propyl acetate, and butyl acetate, ketone solvents, such as methyl ethyl ketone, methyl isobutyl ketone, cyclohexanone, and cyclopentanone, ether solvents, such as tetrahydrofuran, 1,2-dimethoxyethane, and anisole, amide solvents, such as N,N-dimethylformamide and N-methyl-2-pyrrolidone, and propylene glycol monomethyl ether acetate, diethylene glycol monomethyl ether acetate, γ-butyrolactone, and chlorobenzene. One of these may be used alone, or two or more may be used as a mixture, but using any one or more of ketone, ether, ester, and aromatic hydrocarbon solvents is preferred in terms of solution stability.

When adding a solvent, it is preferred to mix it in with stirring using a dispersion mixer. As the dispersion mixer, specifically, a disperser, dispersing machine having stirring blades, such as a propeller or turbine blades, paint shaker, planetary mixer, shaking machine, stirrer, shaker, or rotary evaporator, for example, can be used. Besides these, a sonicator can be used.

The rotational speed for mixing during the addition of a solvent is preferably controlled as needed by means of the mixing device used, but preferably is between 10 rpm and 1000 rpm, more preferably between 50 rpm and 800 rpm, in particular between 150 rpm and 600 rpm to give a uniform solution of the polymerizable liquid crystal composition.

### (Polymerization Inhibitor)

To the composition in the present invention, a known polymerization inhibitor is preferably added. Examples of polymerization initiators include phenolic, quinone, amine, thioether, and nitroso compounds.

### (Orientation Control Agent)

The composition according to the present invention may contain one or more orientation control agents. Examples of orientation control agents that can be contained include salts of alkyl carboxylic acids, alkyl phosphoric acids, alkyl sulfonic acids, fluoroalkyl carboxylic acids, fluoroalkyl phosphoric acids, and fluoroalkyl sulfonic acids, polyoxyethylene derivatives, fluoroalkyl ethylene oxide derivatives, alkylammonium salts, and fluoroalkylammonium salts, and fluorosurfactants in particular are preferred. Specific examples include “Megaface F-251,” “Megaface F-444,” “Megaface F-510,” “Megaface F-552,” “Megaface F-553,” “Megaface F-554,” “Megaface F-555,” “Megaface F-558,” “Megaface F-560,” “Megaface F-561,” “Megaface F-563,” “Megaface F-565,” “Megaface F-570,” “Megaface R-40,” “Megaface R-41,” “Megaface R-43,” and “Megaface R-94” (DIC Corporation) and “FTX-218” (NEOS Co., Ltd.).

Other examples of orientation control agents that can be used include the compounds represented by general formulae (5-1) to (5-4) below, but these are not their only possible structures.

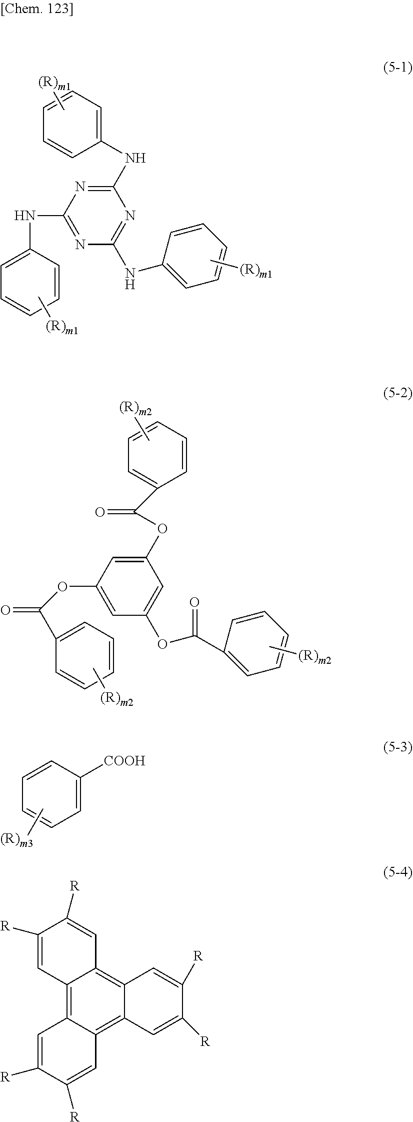

(where Rs each represent a fluorinated or non-fluorinated C1-30 alkoxy group that may be the same as or different from the other(s). In the formulae, m1, m2, and m3 each represent an integer of 1 or more.) (Chain Transfer Agent)

For the composition in the present invention, it is also preferred to add a chain transfer agent to further improve the adhesiveness of optical films made therefrom to a substrate. Examples of chain transfer agents include aromatic hydrocarbons, halogenated hydrocarbons, such as chloroform, carbon tetrachloride, carbon tetrabromide, and bromotrichloromethane, and thiol compounds, such as monothiols, dithiols, trithiols, and tetrathiols, but aromatic hydrocarbons and thiol compounds are preferred.

### (Other Additives)

To adjust physical characteristics, extra additives, for example a polymerizable compound, a thixotropic agent, an ultraviolet absorber, an infrared absorber, an antioxidant, and/or a surface treatment agent, may be added in accordance with the intended purpose(s) in amount(s) that does not affect the coordination potential of the liquid crystal significantly.

A third of the present invention is an optical film that contains a luminescent nanocrystal complex and a binder resin.

The optical film according to the present invention is preferably obtained by applying a composition according to the present invention to a substrate having an aligning capability (e.g., an alignment film) and aligning and polymerizing liquid crystal molecules in the composition according to the present invention while holding a nematic, chiral nematic, smectic, or chiral smectic phase.

The optical film according to the present invention may be obtained by polymerizing a composition according to the present invention and then drawing the resulting material.

### (Method for Producing the Optical Film)

### (Substrate)

The substrate used in the optical film according to the present invention can be of any type as long as it is used in ordinary liquid crystal display devices, displays, optical components, and optical films and is a material that is resistant to heat enough to withstand the heating during the drying of the applied coating of the composition according to the present invention. Examples of such substrates include a glass substrate, a metal substrate, a ceramic substrate, and organic materials, such as a plastic substrate. If the substrate is an organic material in particular, examples include cellulose derivatives, polyolefins, polyesters, polycarbonate, polyacrylate (acrylic resin), polyarylates, polyethersulfones, polyimides, polyphenylene sulfide, polyphenylene ethers, nylon, and polystyrene. A plastic substrate, for example of polyester, polyacrylate, a polyolefin, a cellulose derivative, polyarylate, or polycarbonate, is particularly preferred, and a polyacrylate, polyolefin, cellulose derivative, or similar substrate is more preferred. It is particularly preferred to use a COP (cycloolefin polymer) as a polyolefin, TAC (triacetyl cellulose) as a cellulose derivative, or PMMA (polymethyl methacrylate) as a polyacrylate. The shape of the substrate may be a flat plate or may alternatively be curved. These substrates may optionally have an electrode layer, an antireflection capability, and/or a reflection capability.

These substrates may have their surface treated to improve the ease of coating and the adhesion of the composition according to the present invention. Examples of surface treatments include ozonation, plasma treatment, corona treatment, and silane coupling treatment. An organic, inorganic-oxide, or metal thin film, for example, may be formed on the surface of the substrate by a method, such as deposition, to adjust optical transmittance and reflectance, or the substrate may be, for example, a pickup lens, rod lens, optical disk, retardation film, diffuser film, or color filter to give it optical added value. A pickup lens, a retardation film, a diffuser film, and a color filter, which will be given higher added values, are particularly preferred.

### (Aligning)

The substrate has usually been treated for aligning, or there may be an alignment film thereon, so that the composition according to the present invention will reach a predetermined alignment in the dried coating of the composition. Examples of aligning treatments include drawing, rubbing, irradiation with polarized ultraviolet and visible light, and ion-beam treatment. If an alignment film is used, the alignment film is a known and commonly used one. Examples of such alignment films include polyimides, polysiloxane, polyamides, polyvinyl alcohol, polycarbonate, polystyrene, polyphenylene ethers, polyarylates, polyethylene terephthalate, polyethersulfones, epoxy resin, epoxy acrylate resin, acrylic resin, and compounds such as coumarin, chalcone, cinnamate, fulgide, anthraquinone, azo, and arylethene compounds. For those compounds that are aligned by rubbing, materials whose crystallization is promoted by the aligning treatment or a heating step following the aligning treatment are preferred. Among those compounds that are aligned except by rubbing, it is preferred to use a material that is aligned by light.

### (Application)

The coating method used to obtain the optical film according to the present invention can be a known and commonly used method, such as the use of an applicator, bar coating, spin coating, roll coating, direct gravure coating, reverse gravure coating, flexo coating, ink jetting, die coating, cap coating, dip coating, or slit coating. The applied composition is dried as necessary.

### (Method for Polymerization)

The polymerization of the composition according to the present invention can be performed by, for example, irradiation with active energy radiation or thermal polymerization. Requiring no heating and allowing the reaction to proceed at room temperature, irradiation with active energy radiation is preferred. In particular, irradiation with light, for example ultraviolet radiation, is preferred.

### EXAMPLES

The following describes the present invention by synthesis examples, examples, and comparative examples, but, needless to say, the present invention is not limited to these. “Parts” and “%” are by mass unless otherwise specified.

### Example 1

### “Synthesis of a Luminescent Nanocrystal Complex”

### (Core-Shell Luminescent Nanocrystals)

### ““Synthesis by Ligand Exchange””

### Green Light-Emitting

To a solution of 0.15 mg of oleylamine-coordinated InP/Zn nanoparticles (NN labs; emission peak, 515 to 545 nm) in 10 ml of toluene, 0.5 mg of the compound below was added as a ligand (B-S3-C5) to replace the oleylamine.

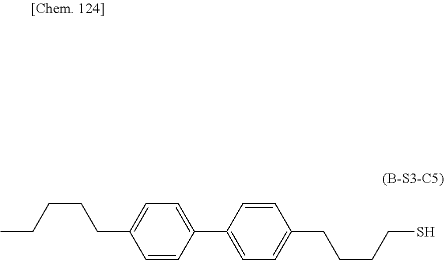 [SMILES: CCCCCc1ccc(-c2ccc(CCCCS)cc2)cc1]

After the addition, the solution was stirred at 50° C. for 1 hour, and the solution that completed the reaction was centrifuged (5000 rpm, 1 hour) with 30 ml of ethanol. The supernatant was removed, and the precipitate was dispersed again in 10 ml of toluene in a nitrogen atmosphere to give an InP/ZnS luminescent nanocrystal complex surface-modified with a ligand (B-S3-C5).

### Example 2

### Red Light-Emitting

An InP/ZnS luminescent nanocrystal complex surface-modified with a ligand (B-S3-C5) was obtained as in Example 1 except that oleylamine-coordinated InP/Zn nanoparticles with an emission peak at 635 to 665 nm (NN labs) were used.

In the subsequent Examples, a ligand-modified InP/ZnS luminescent nanocrystal complex that emits green or red light was produced as in Example 1 or 2 except that a different ligand was used. Specific details are as follows.

### Example 3

### Green Light-Emitting InP/ZnS Luminescent Nanocrystal Complex (Ligand: B-S1-C5)

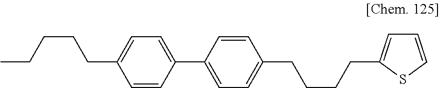 [SMILES: CCCCCc1ccc(-c2ccc(CCCCc3cccs3)cc2)cc1]

### Example 4

### Red Light-Emitting InP/ZnS Luminescent Nanocrystal Complex (Ligand: B-S1-C5)

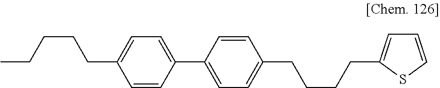 [SMILES: CCCCCc1ccc(-c2ccc(CCCCc3cccs3)cc2)cc1]

### Example 5

### Green Light-Emitting InP/ZnS Luminescent Nanocrystal Complex (Ligand: B-S2-C5)

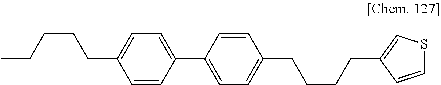 [SMILES: CCCCCc1ccc(-c2ccc(CCCCc3ccsc3)cc2)cc1]

### Example 6

### Red Light-Emitting InP/ZnS Luminescent Nanocrystal Complex (Ligand: B-S2-C5)

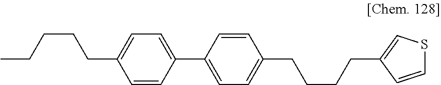 [SMILES: CCCCCc1ccc(-c2ccc(CCCCc3ccsc3)cc2)cc1]

### Example 7

### Green Light-Emitting InP/ZnS Luminescent Nanocrystal Complex (Ligand: B-P1-C5)

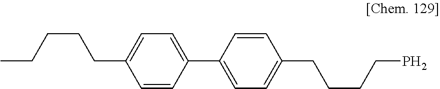

### Example 8

### Red Light-Emitting InP/ZnS Luminescent Nanocrystal Complex (Ligand: B-P1-C5)

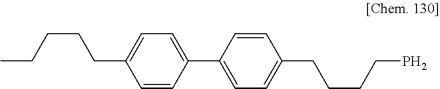

### Example 9

### Green Light-Emitting InP/ZnS Luminescent Nanocrystal Complex (Ligand: B-P2-C5)

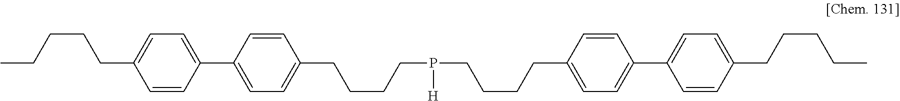 [SMILES: CCCCCc1ccc(-c2ccc(CCCCPCCCCc3ccc(-c4ccc(CCCCC)cc4)cc3)cc2)cc1]

### Example 10

### Red Light-Emitting InP/ZnS Luminescent Nanocrystal Complex (Ligand: B-P2-C5)

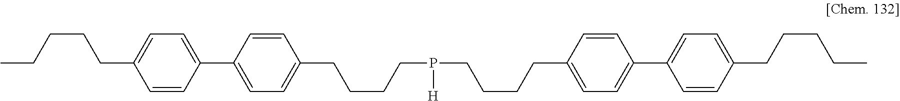 [SMILES: CCCCCc1ccc(-c2ccc(CCCCPCCCCc3ccc(-c4ccc(CCCCC)cc4)cc3)cc2)cc1]

### Example 11

### Green Light-Emitting InP/ZnS Luminescent Nanocrystal Complex (Ligand: B-P3-C5)

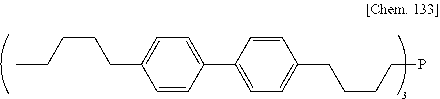

### Example 12

### Red Light-Emitting InP/ZnS Luminescent Nanocrystal Complex (Ligand: B-P3-C5)

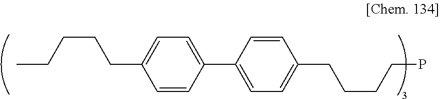

### Example 13

### Green Light-Emitting InP/ZnS Luminescent Nanocrystal Complex (Ligand: B-P4-C5)

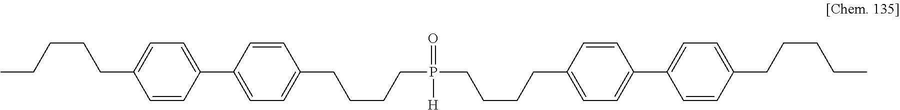 [SMILES: CCCCCc1ccc(-c2ccc(CCCC[PH](=O)CCCCc3ccc(-c4ccc(CCCCC)cc4)cc3)cc2)cc1]

### Example 14

### Red Light-Emitting InP/ZnS Luminescent Nanocrystal Complex (Ligand: B-P4-C5)

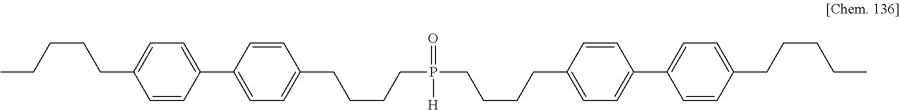 [SMILES: CCCCCc1ccc(-c2ccc(CCCC[PH](=O)CCCCc3ccc(-c4ccc(CCCCC)cc4)cc3)cc2)cc1]

### Example 15

### Green Light-Emitting InP/ZnS Luminescent Nanocrystal Complex (Ligand: B-P5-C5)

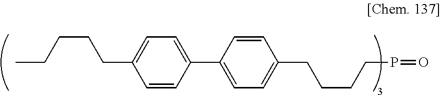

### Example 16

### Red Light-Emitting InP/ZnS Luminescent Nanocrystal Complex (Ligand: B-P5-C5)

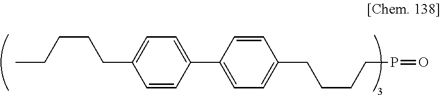

### Example 17

### Green Light-Emitting InP/ZnS Luminescent Nanocrystal Complex (Ligand: B-S1-VY)

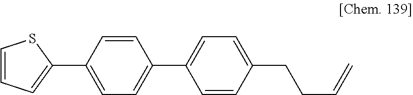 [SMILES: C=CCCc1ccc(-c2ccc(-c3cccs3)cc2)cc1]

### Example 18

### Red Light-Emitting InP/ZnS Luminescent Nanocrystal Complex (Ligand: B-S1-VY)

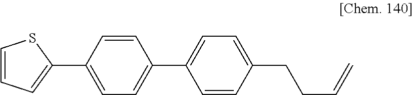 [SMILES: C=CCCc1ccc(-c2ccc(-c3cccs3)cc2)cc1]

### Example 19

### Green Light-Emitting InP/ZnS Luminescent Nanocrystal Complex (Ligand: B-S2-VY)

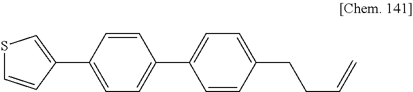 [SMILES: C=CCCc1ccc(-c2ccc(-c3ccsc3)cc2)cc1]

### Example 20

### Red Light-Emitting InP/ZnS Luminescent Nanocrystal Complex (Ligand: B-S2-VY)

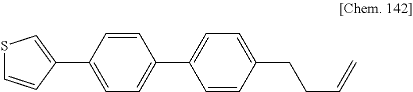 [SMILES: C=CCCc1ccc(-c2ccc(-c3ccsc3)cc2)cc1]

### Example 21

### Green Light-Emitting InP/ZnS Luminescent Nanocrystal Complex (Ligand: B-S3-VY)

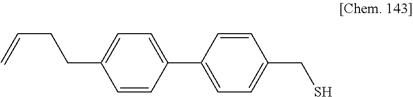 [SMILES: C=CCCc1ccc(-c2ccc(CS)cc2)cc1]

### Example 22

### Red Light-Emitting InP/ZnS Luminescent Nanocrystal Complex (Ligand: B-S3-VY)

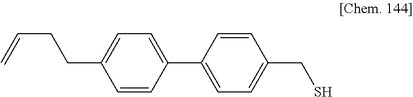 [SMILES: C=CCCc1ccc(-c2ccc(CS)cc2)cc1]

### Example 23

### Green Light-Emitting InP/ZnS Luminescent Nanocrystal Complex (Ligand: B-P3-VY)

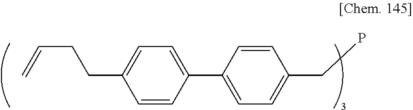

### Example 24

### Red Light-Emitting InP/ZnS Luminescent Nanocrystal Complex (Ligand: B-P3-VY)

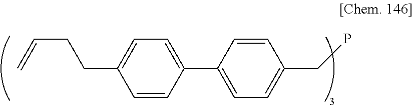

### Example 25

### Green Light-Emitting InP/ZnS Luminescent Nanocrystal Complex (Ligand: B-S1-AC)

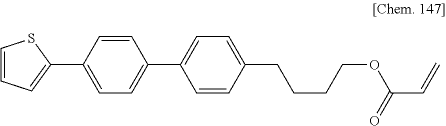 [SMILES: C=CC(=O)OCCCCc1ccc(-c2ccc(-c3cccs3)cc2)cc1]

### Example 26

### Red Light-Emitting InP/ZnS Luminescent Nanocrystal Complex (Ligand: B-S1-AC)

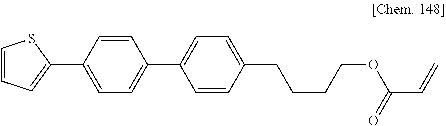 [SMILES: C=CC(=O)OCCCCc1ccc(-c2ccc(-c3cccs3)cc2)cc1]

### Example 27

### Green Light-Emitting InP/ZnS Luminescent Nanocrystal Complex (Ligand: B-S2-AC)

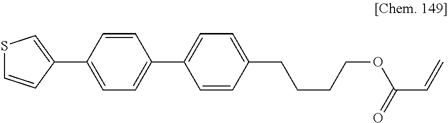 [SMILES: C=CC(=O)OCCCCc1ccc(-c2ccc(-c3ccsc3)cc2)cc1]

### Example 28

### Red Light-Emitting InP/ZnS Luminescent Nanocrystal Complex (Ligand: B-S2-AC)

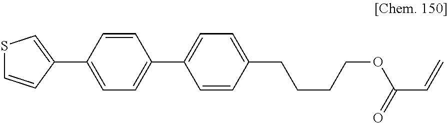 [SMILES: C=CC(=O)OCCCCc1ccc(-c2ccc(-c3ccsc3)cc2)cc1]

### Example 29

### Green Light-Emitting InP/ZnS Luminescent Nanocrystal Complex (Ligand: B-P3-AC)

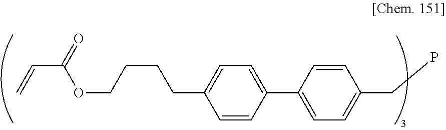

### Example 30

### Red Light-Emitting InP/ZnS Luminescent Nanocrystal Complex (Ligand: B-P3-AC)

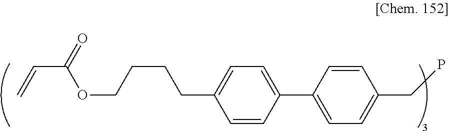

### Example 31

### Green Light-Emitting InP/ZnS Luminescent Nanocrystal Complex (Ligand: T-S1-C5)

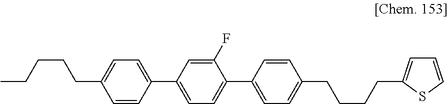 [SMILES: CCCCCc1ccc(-c2ccc(-c3ccc(CCCCc4cccs4)cc3)c(F)c2)cc1]

### Example 32

### Red Light-Emitting InP/ZnS Luminescent Nanocrystal Complex (Ligand: T-S1-C5)

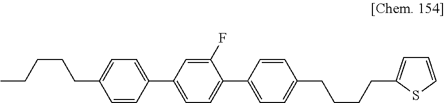 [SMILES: CCCCCc1ccc(-c2ccc(-c3ccc(CCCCc4cccs4)cc3)c(F)c2)cc1]

### Example 33

### Green Light-Emitting InP/ZnS Luminescent Nanocrystal Complex (Ligand: T-S2-C5)

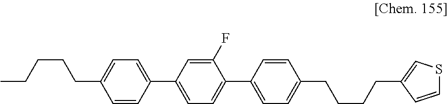 [SMILES: CCCCCc1ccc(-c2ccc(-c3ccc(CCCCc4ccsc4)cc3)c(F)c2)cc1]

### Example 34

### Red Light-Emitting InP/ZnS Luminescent Nanocrystal Complex (Ligand: T-S2-C5)

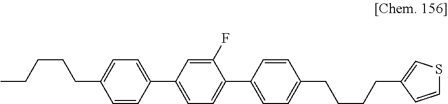 [SMILES: CCCCCc1ccc(-c2ccc(-c3ccc(CCCCc4ccsc4)cc3)c(F)c2)cc1]

### Example 35

### Green Light-Emitting InP/ZnS Luminescent Nanocrystal Complex (Ligand: T-S3-C5)

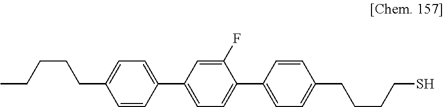 [SMILES: CCCCCc1ccc(-c2ccc(-c3ccc(CCCCS)cc3)c(F)c2)cc1]

### Example 36

### Red Light-Emitting InP/ZnS Luminescent Nanocrystal Complex (Ligand: T-S3-C5)

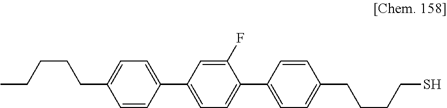 [SMILES: CCCCCc1ccc(-c2ccc(-c3ccc(CCCCS)cc3)c(F)c2)cc1]

### Example 37

### Green Light-Emitting InP/ZnS Luminescent Nanocrystal Complex (Ligand: T-P1-C5)

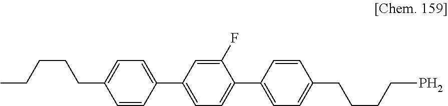

### Example 38

### Red Light-Emitting InP/ZnS Luminescent Nanocrystal Complex (Ligand: T-P1-C5)

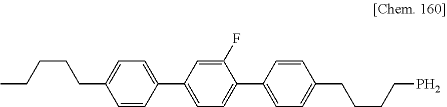

### Example 39

### Green Light-Emitting InP/ZnS Luminescent Nanocrystal Complex (Ligand: T-P2-C5)

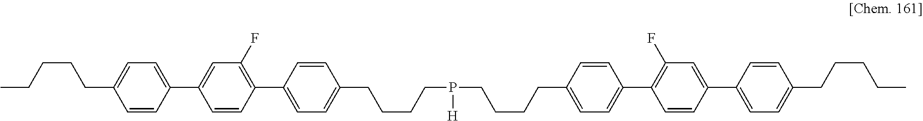 [SMILES: CCCCCc1ccc(-c2ccc(-c3ccc(CCCCPCCCCc4ccc(-c5ccc(-c6ccc(CCCCC)cc6)cc5F)cc4)cc3)c(F)c2)cc1]

### Example 40

### Red Light-Emitting InP/ZnS Luminescent Nanocrystal Complex (Ligand: T-P2-C5)

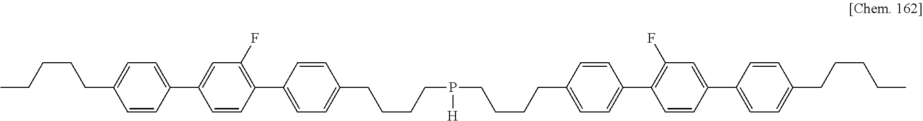 [SMILES: CCCCCc1ccc(-c2ccc(-c3ccc(CCCCPCCCCc4ccc(-c5ccc(-c6ccc(CCCCC)cc6)cc5F)cc4)cc3)c(F)c2)cc1]

### Example 41

### Green Light-Emitting InP/ZnS Luminescent Nanocrystal Complex (Ligand: T-P3-C5)

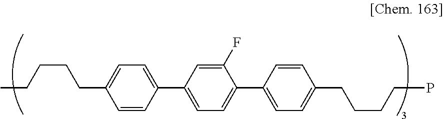

### Example 42

### Red Light-Emitting InP/ZnS Luminescent Nanocrystal Complex (Ligand: T-P3-C5)

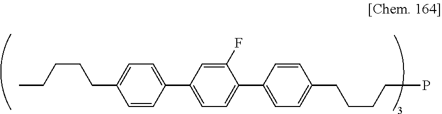

### Example 43

### Green Light-Emitting InP/ZnS Luminescent Nanocrystal Complex (T-P4-C5)

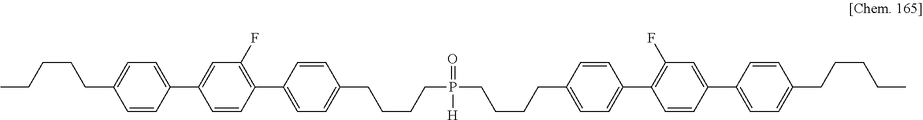 [SMILES: CCCCCc1ccc(-c2ccc(-c3ccc(CCCC[PH](=O)CCCCc4ccc(-c5ccc(-c6ccc(CCCCC)cc6)cc5F)cc4)cc3)c(F)c2)cc1]

### Example 44

### Red Light-Emitting InP/ZnS Luminescent Nanocrystal Complex (T-P4-C5)

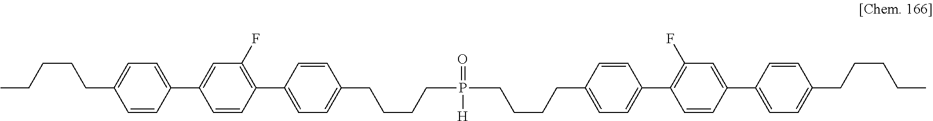 [SMILES: CCCCCc1ccc(-c2ccc(-c3ccc(CCCC[PH](=O)CCCCc4ccc(-c5ccc(-c6ccc(CCCCC)cc6)cc5F)cc4)cc3)c(F)c2)cc1]

### Example 45

### Green Light-Emitting InP/ZnS Luminescent Nanocrystal Complex (Ligand: T-P5-C5)

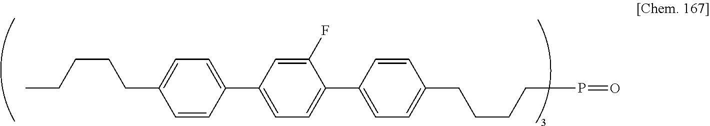

### Example 46

### Red Light-Emitting InP/ZnS Luminescent Nanocrystal Complex (Ligand: T-P5-C5)

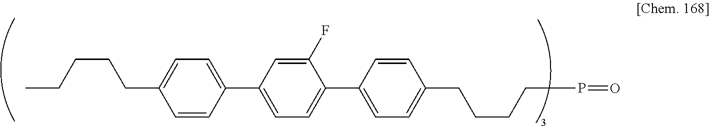

### Synthesis by ““Coordinating a Surface-Modifying Compound During the Synthesis of Luminescent Nanocrystals (Capping)””

### Example 47

### Green Light-Emitting

A surface-modified nanophosphor coordinated with a ligand according to the present invention was synthesized as in Japanese Unexamined Patent Application Publication (Translation of PCT Application) No. 2011-530187. Specific details are as follows. Indium phosphide core particles (0.155 g in 4.4 ml of dibutyl sebacate) were put into a three-neck flask (100 ml) heated and vacuum-dried beforehand, and then the flask was evacuated at 100° C. for one and a half hours. After cooling to room temperature, the flask was purged with nitrogen. Then zinc acetate (0.07483 g) and the ligand (=surface-modifying compound) (B-H2-C5, 0.5243 g) were added,

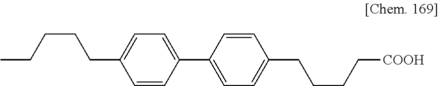 [SMILES: CCCCCc1ccc(-c2ccc(CCCCC(=O)O)cc2)cc1]

the mixture was degassed at 55° C. for 1 hour, and the flask was purged with nitrogen. After heating to 190° C., tert-nonyl mercaptan (0.29 ml) was added dropwise and allowed to react at 190° C. Samples were taken while the reaction was proceeding, and their UV-vis spectrum was measured. After absorption was observed near 530 nm, the reaction mixture was cooled to room temperature.

In a nitrogen atmosphere, ethyl acetate was added, and the mixture was centrifuged to isolate InP/ZnS core-shell nanoparticles modified with a ligand (B-H2-C5). The particles were precipitated with acetonitrile and separated by centrifugation, and then the particles were dispersed in toluene, precipitated again with acetonitrile, and separated by centrifugation. This dispersion-precipitation using toluene and acetonitrile was repeated a total of four times, and finally toluene was dispersed to give an InP/ZnS luminescent nanocrystal complex surface-modified with a ligand (B-H2-C5).

### Example 48

### Red Light-Emitting

An InP/ZnS luminescent nanocrystal complex surface-modified with a ligand (B-H2-C5) was obtained in the same way as in Example 47 except that samples were taken while the reaction was proceeding, and the reaction was continued until the UV-vis spectrum of the samples had absorption near 650 nm.

In the subsequent Examples, a ligand-modified InP/ZnS luminescent nanocrystal complex that emits green or red light was produced in the same way as in Example 47 or 48 except that a different ligand was used. Specific details are as follows.

### Example 49

### Green Light-Emitting InP/ZnS Luminescent Nanocrystal Complex (Ligand: B-N1-C5)

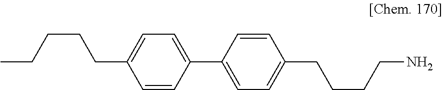 [SMILES: CCCCCc1ccc(-c2ccc(CCCCN)cc2)cc1]

### Example 50

### Red Light-Emitting InP/ZnS Luminescent Nanocrystal Complex (Ligand: B-N1-C5)

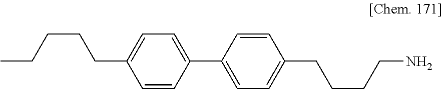 [SMILES: CCCCCc1ccc(-c2ccc(CCCCN)cc2)cc1]

### Example 51

### Green Light-Emitting InP/ZnS Luminescent Nanocrystal Complex (Ligand: B-N2-C5)

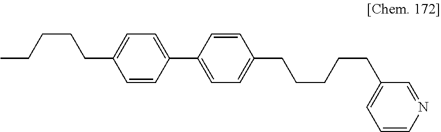 [SMILES: CCCCCc1ccc(-c2ccc(CCCCCc3cccnc3)cc2)cc1]

### Example 52

### Red Light-Emitting InP/ZnS Luminescent Nanocrystal Complex (Ligand: B-N2-C5)

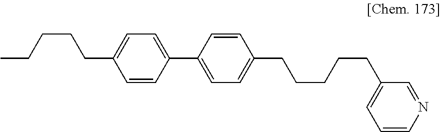 [SMILES: CCCCCc1ccc(-c2ccc(CCCCCc3cccnc3)cc2)cc1]

### Example 53

### Green Light-Emitting InP/ZnS Luminescent Nanocrystal Complex (Ligand: B-H1-C5)

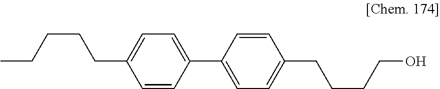 [SMILES: CCCCCc1ccc(-c2ccc(CCCCO)cc2)cc1]

### Example 54

### Red Light-Emitting InP/ZnS Luminescent Nanocrystal Complex (Ligand: B-H1-C5)

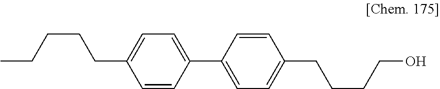 [SMILES: CCCCCc1ccc(-c2ccc(CCCCO)cc2)cc1]

### Example 55

### Green Light-Emitting InP/ZnS Luminescent Nanocrystal Complex (Ligand: B-N2-VY)

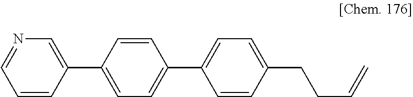 [SMILES: C=CCCc1ccc(-c2ccc(-c3cccnc3)cc2)cc1]

### Example 56

### Red Light-Emitting InP/ZnS Luminescent Nanocrystal Complex (Ligand: B-N2-VY)

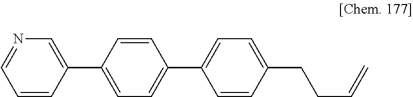 [SMILES: C=CCCc1ccc(-c2ccc(-c3cccnc3)cc2)cc1]

### Example 57

### Green Light-Emitting InP/ZnS Luminescent Nanocrystal Complex (Ligand: B-N3-VY)

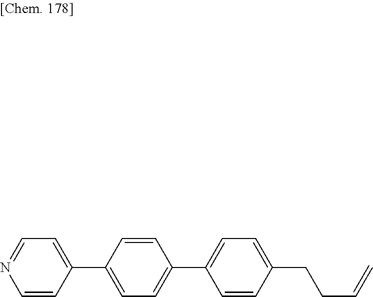 [SMILES: C=CCCc1ccc(-c2ccc(-c3ccncc3)cc2)cc1]

### Example 58

### Red Light-Emitting InP/ZnS Luminescent Nanocrystal Complex (Ligand: B-N3-VY)

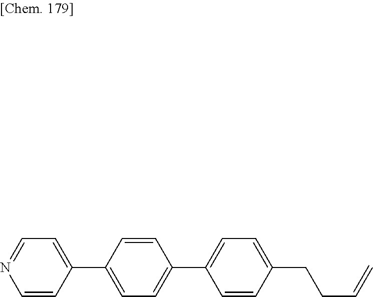 [SMILES: C=CCCc1ccc(-c2ccc(-c3ccncc3)cc2)cc1]

### Example 59

### Green Light-Emitting InP/ZnS Luminescent Nanocrystal Complex (Ligand: B-H1-VY)

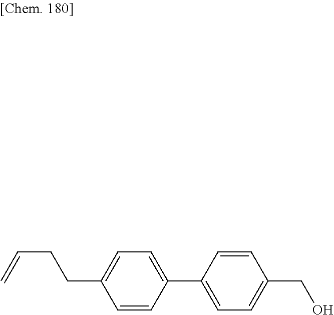 [SMILES: C=CCCc1ccc(-c2ccc(CO)cc2)cc1]

### Example 60

### Red Light-Emitting InP/ZnS Luminescent Nanocrystal Complex (Ligand: B-H1-VY)

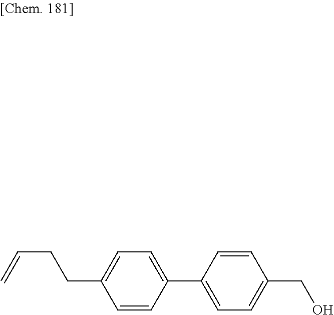 [SMILES: C=CCCc1ccc(-c2ccc(CO)cc2)cc1]

### Example 61

### Green Light-Emitting InP/ZnS Luminescent Nanocrystal Complex (Ligand: B-H2-VY)

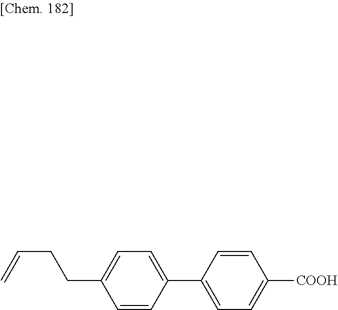 [SMILES: C=CCCc1ccc(-c2ccc(C(=O)O)cc2)cc1]

### Example 62

### Red Light-Emitting InP/ZnS Luminescent Nanocrystal Complex (Ligand: B-H2-VY)

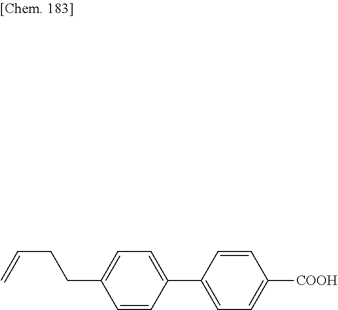 [SMILES: C=CCCc1ccc(-c2ccc(C(=O)O)cc2)cc1]

### Example 63

### Green Light-Emitting InP/ZnS Luminescent Nanocrystal Complex (Ligand: T-N1-C5)

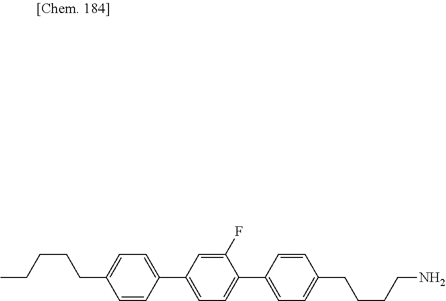 [SMILES: CCCCCc1ccc(-c2ccc(-c3ccc(CCCCN)cc3)c(F)c2)cc1]

### Example 64

### Red Light-Emitting InP/ZnS Luminescent Nanocrystal Complex (Ligand: T-N1-C5)

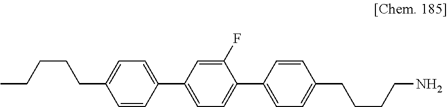 [SMILES: CCCCCc1ccc(-c2ccc(-c3ccc(CCCCN)cc3)c(F)c2)cc1]

### Example 65

### Green Light-Emitting InP/ZnS Luminescent Nanocrystal Complex (Ligand: T-N2-C5)

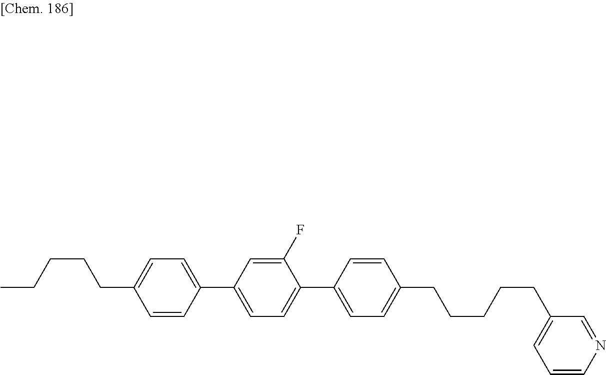 [SMILES: CCCCCc1ccc(-c2ccc(-c3ccc(CCCCCc4cccnc4)cc3)c(F)c2)cc1]

### Example 66

### Red Light-Emitting InP/ZnS Luminescent Nanocrystal Complex (Ligand: T-N2-C5)

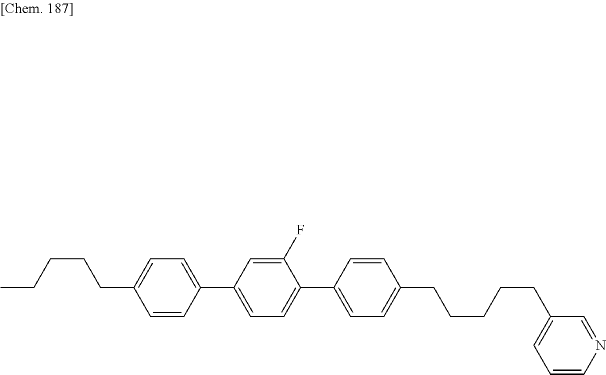 [SMILES: CCCCCc1ccc(-c2ccc(-c3ccc(CCCCCc4cccnc4)cc3)c(F)c2)cc1]

### Example 67

### Green Light-Emitting InP/ZnS Luminescent Nanocrystal Complex (Ligand: T-H1-C5)

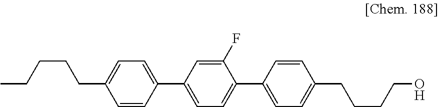 [SMILES: CCCCCc1ccc(-c2ccc(-c3ccc(CCCCO)cc3)c(F)c2)cc1]

### Example 68

### Red Light-Emitting InP/ZnS Luminescent Nanocrystal Complex (Ligand: T-H1-C5)

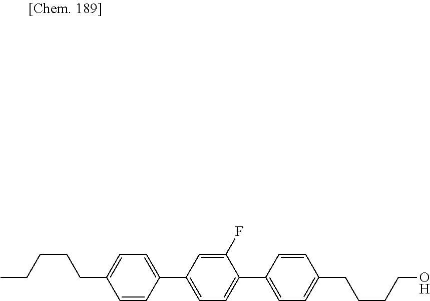 [SMILES: CCCCCc1ccc(-c2ccc(-c3ccc(CCCCO)cc3)c(F)c2)cc1]

### Example 69

### Green Light-Emitting InP/ZnS Luminescent Nanocrystal Complex (Ligand: T-H2-C5)

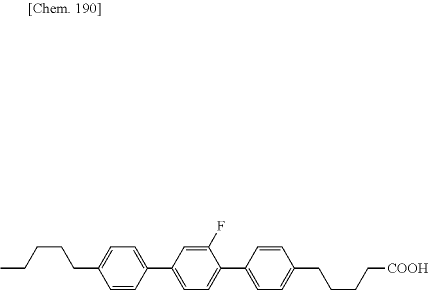 [SMILES: CCCCCc1ccc(-c2ccc(-c3ccc(CCCCC(=O)O)cc3)c(F)c2)cc1]

### Example 70

### Red Light-Emitting InP/ZnS Luminescent Nanocrystal Complex (Ligand: T-H2-C5)

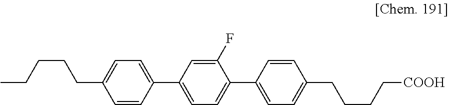 [SMILES: CCCCCc1ccc(-c2ccc(-c3ccc(CCCCC(=O)O)cc3)c(F)c2)cc1]

### Example 71

### Core-Type

Core-type fluorescent nanoparticles having a mesogen-structured ligand were produced in the same way as the core-shell nanocrystals were produced by ligand exchange. The details are as follows.

### ““Synthesis by Ligand Exchange””

### Green Light-Emitting

To a solution of 0.15 mg of octadecylamine-coordinated CdSe nanoparticles (NN labs; emission peak, 515 to 545 nm) in 10 ml of toluene, 0.5 mg of the compound below was added as a ligand (B-S3-C5) to replace the octadecylamine.

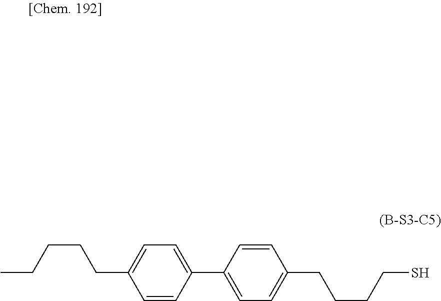 [SMILES: CCCCCc1ccc(-c2ccc(CCCCS)cc2)cc1]

After the addition, the solution was stirred at 50° C. for 1 hour, and the solution that completed the reaction was centrifuged (5000 rpm, 1 hour) with 30 ml of ethanol. The supernatant was removed, and the precipitate was dispersed again in 10 ml of toluene in a nitrogen atmosphere to give a CdSe luminescent nanocrystal complex surface-modified with a ligand (B-S3-C5).

### Example 72

### Red Light-Emitting

A CdSe luminescent nanocrystal complex surface-modified with a ligand (B-S3-C5) was obtained as in Example 71 except that octadecylamine-coordinated CdSe nanoparticles with an emission peak at 635 to 665 nm (NN labs) were used.

### Example 73

### Rod-Shaped

Rod-shaped fluorescent nanoparticles having a mesogen-structure ligand were produced in the same way as the core-shell nanocrystals were produced by ligand exchange. The trioctylphosphine oxide (TOPO)-coordinated rod-shaped fluorescent SeCd nanoparticles were produced as in Nauture, Vol. 404, 59-61. The details are as follows.

### ““Synthesis by Ligand Exchange””

### Red Light-Emitting

To a solution of 0.15 mg of TOPO-coordinated rod-shaped CdSe nanoparticles (emission peak, 620 nm) in 10 ml of toluene, 0.5 mg of the compound below was added as a ligand (B-S3-C5) to replace the TOPO.

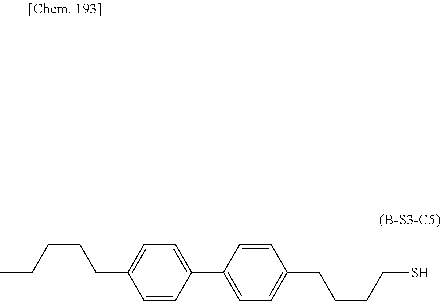 [SMILES: CCCCCc1ccc(-c2ccc(CCCCS)cc2)cc1]

After the addition, the solution was stirred at 50° C. for 1 hour, and the solution that completed the reaction was centrifuged (5000 rpm, 1 hour) with 30 ml of ethanol. The supernatant was removed, and the precipitate was dispersed again in 10 ml of toluene in a nitrogen atmosphere to give a rod-shaped CdSe luminescent nanocrystal complex surface-modified with a ligand (B-S3-C5).

The surface-modifying compounds (=ligands) used in the above Examples were synthesized in accordance with the following synthetic schemes. Examples 74 to 110 below describe how the surface-modifying compounds were synthesized.

### Example 74

### Synthesis of Ligand: B-S1-C5

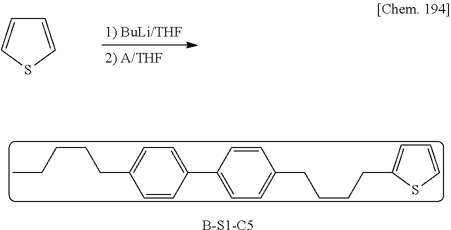

### Example 75

### Synthesis of Ligand: B-S2-C5

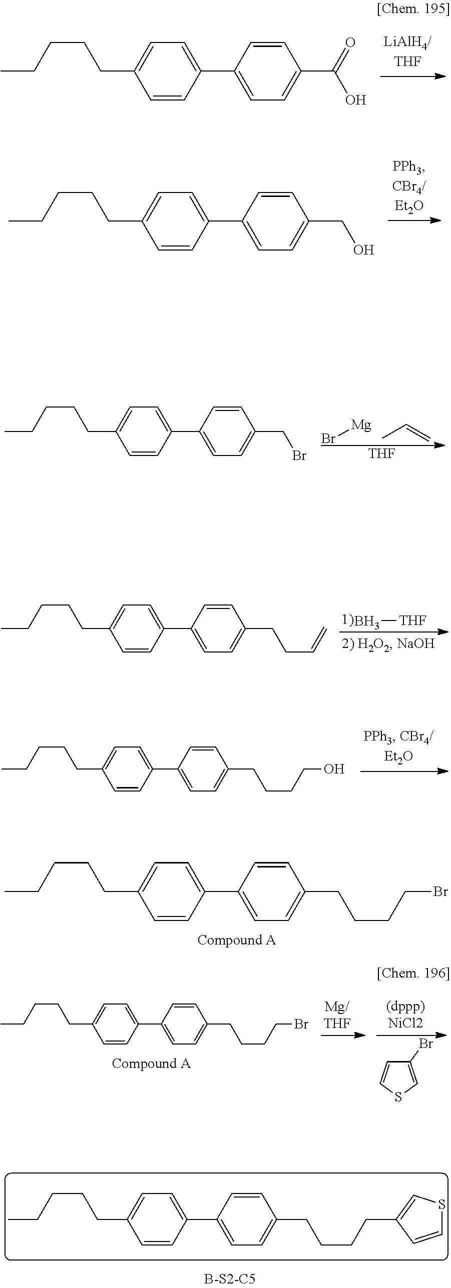

### Example 76

### Synthesis of Ligand: B-S3-C5

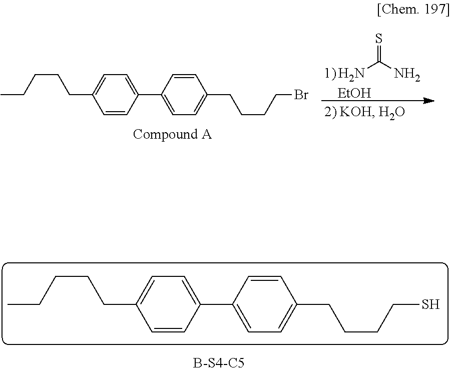

### Example 77

### Synthesis of Ligand: B-P1-C5

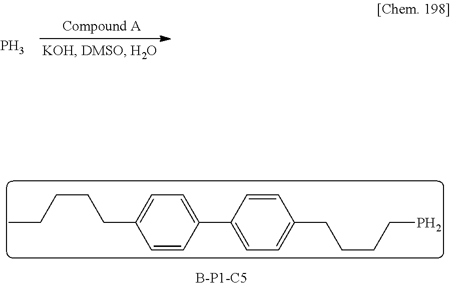

### Example 78

### Synthesis of Ligand: B-P2-C5

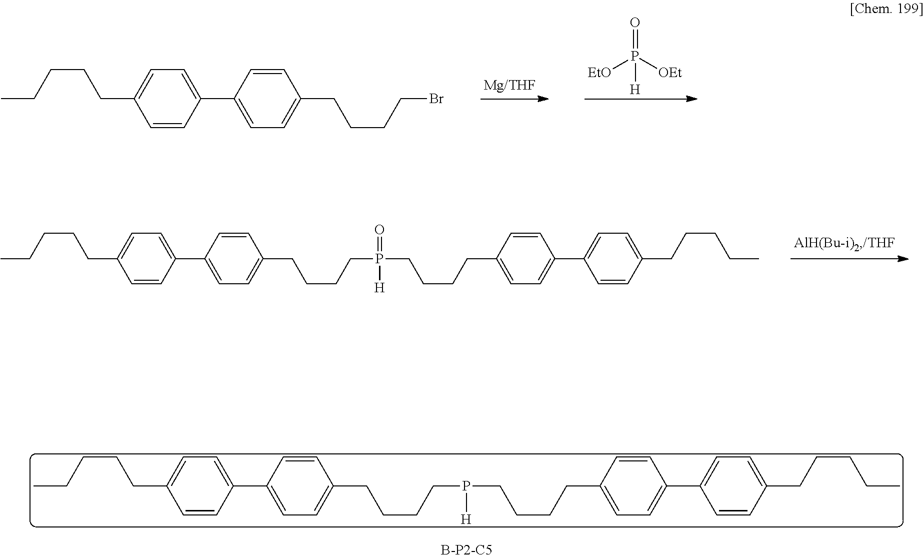

### Example 79

### Synthesis of Ligand: B-P3-C5

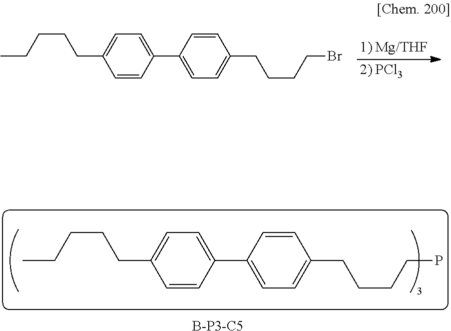

### Example 80

### Synthesis of Ligand: B-P4-C5

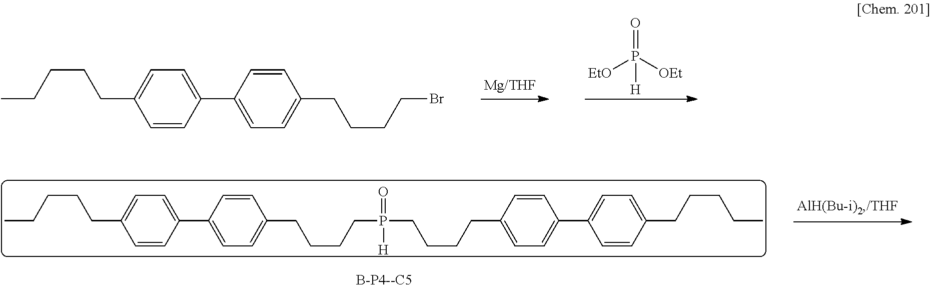 [SMILES: CCCCCc1ccc(-c2ccc(CCCCBr)cc2)cc1 ; CCO[PH](=O)OCC ; CCCCCc1ccc(-c2ccc(CCCC[PH](=O)CCCCc3ccc(-c4ccc(CCCCC)cc4)cc3)cc2)cc1]

### Example 81

### Synthesis of Ligand: B-P5-C5

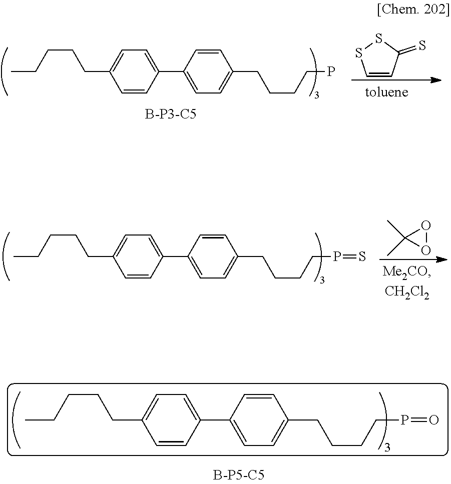

### Example 82

### Synthesis of Ligand: B-N1-C5

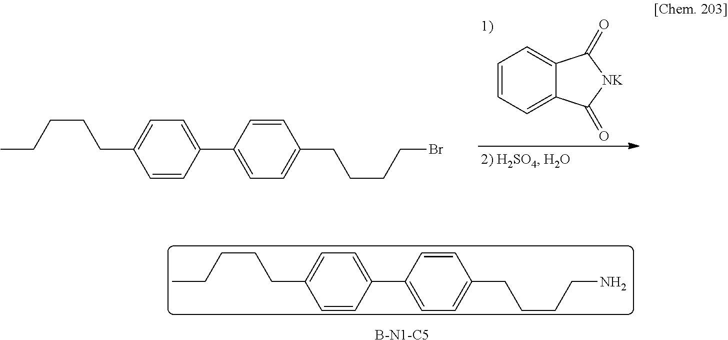

### Example 83

### Synthesis of Ligand: B-N4-C5

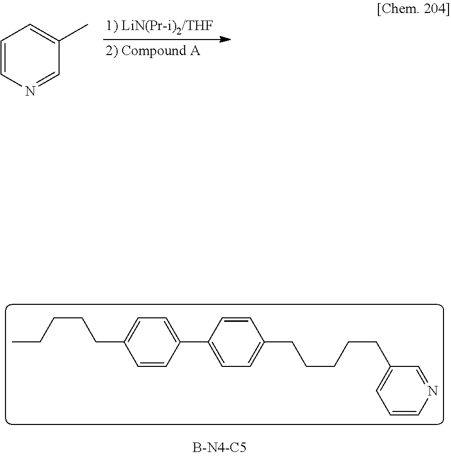

### Example 84

### Synthesis of Ligand: B-H1-C5

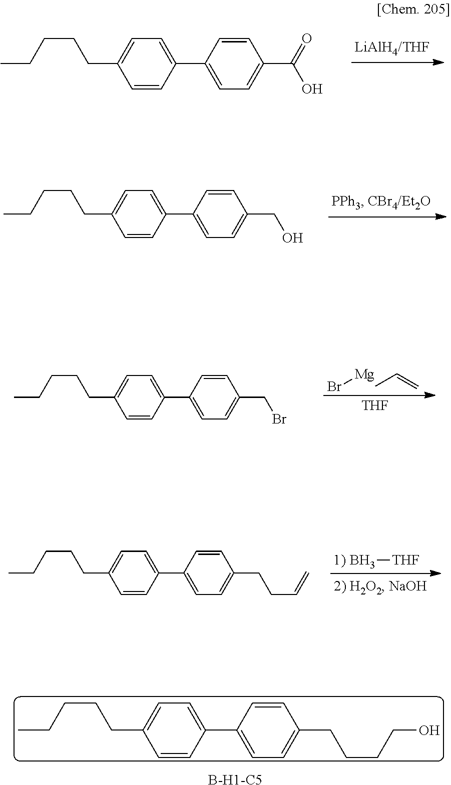

### Example 85

### Synthesis of Ligand: B-H2-C5

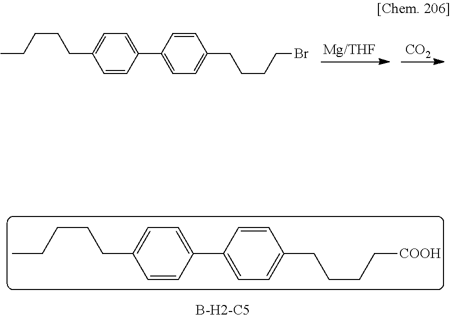 [SMILES: CCCCCc1ccc(-c2ccc(CCCCBr)cc2)cc1 ; "CO2" ; CCCCCc1ccc(-c2ccc(CCCCC(=O)O)cc2)cc1 ; "B-H2-C5"]

### Example 86

### Synthesis of Ligand: B-S1-VY

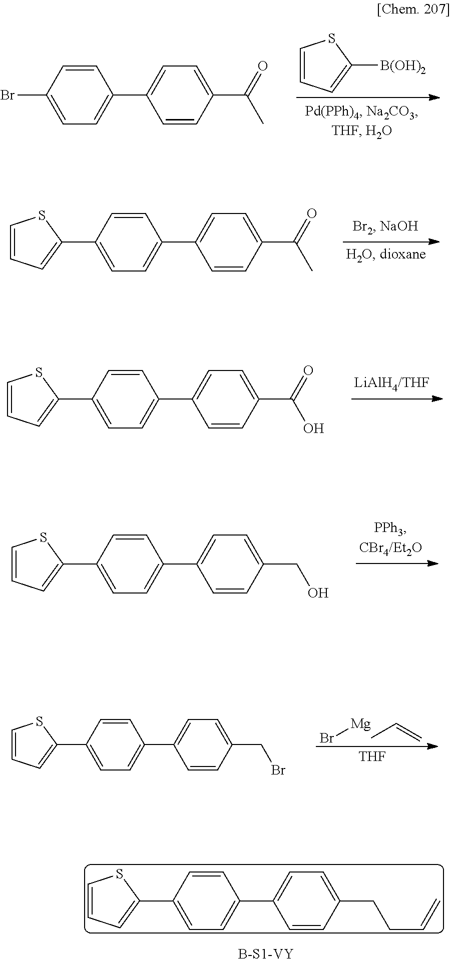

### Example 87

### Synthesis of Ligand: B-S2-C5

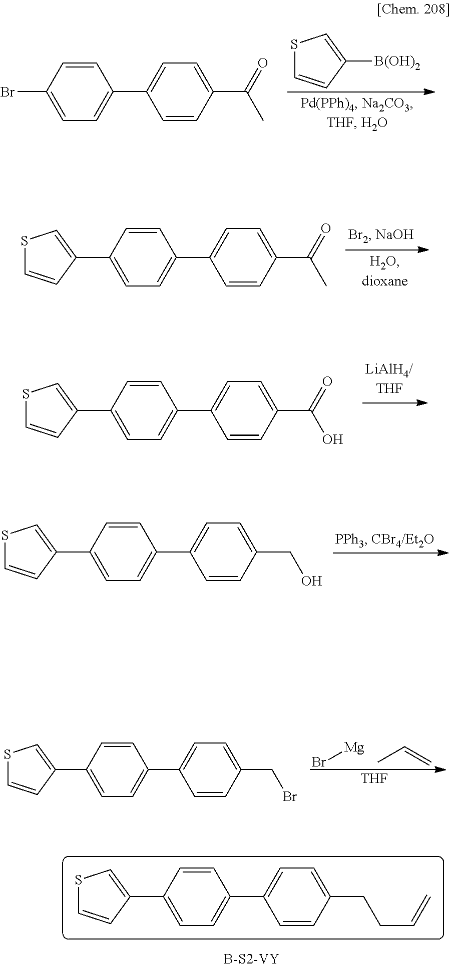

### Example 88

### Synthesis of Ligand: B-S4-VY

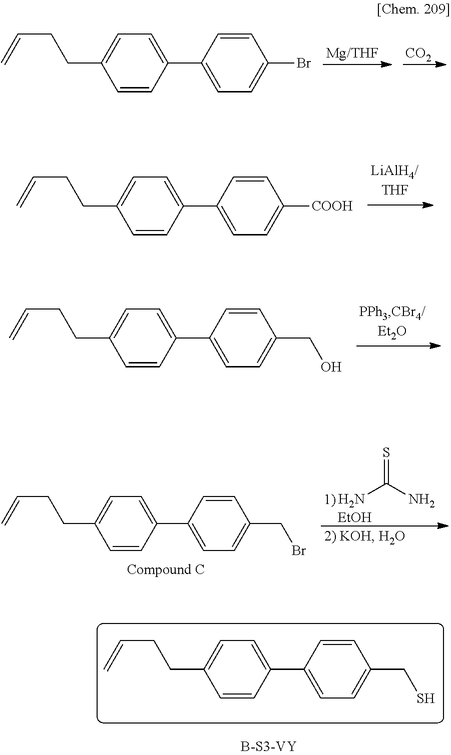

### Example 89

### Synthesis of Ligand: B-P3-VY

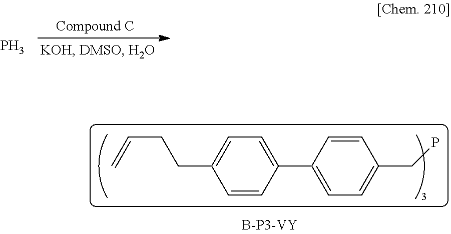

### Example 90

### Synthesis of Ligand: B-N2-VY

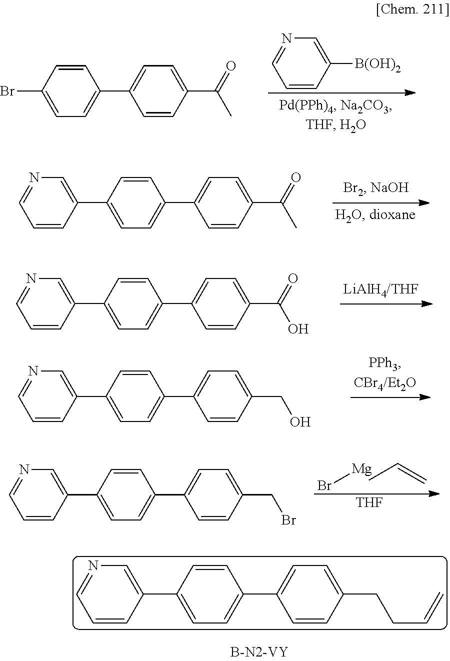

### Example 91

### Synthesis of Ligand: B-N3-VY

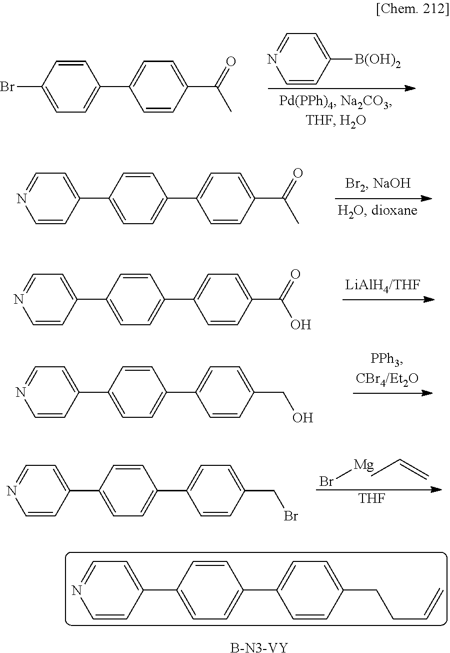

### Example 92

### Synthesis of Ligand: B-H1-VY

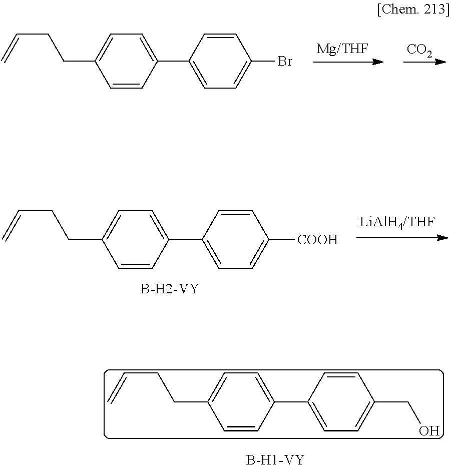

### Example 93

### Synthesis of Ligand: B-H2-VY

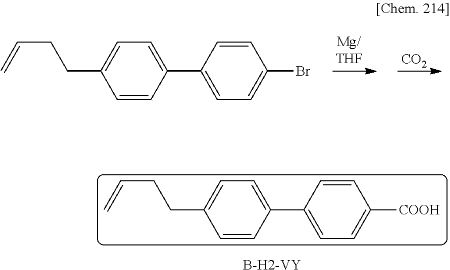

### Example 94

### Synthesis of Ligand: B-S1-AC

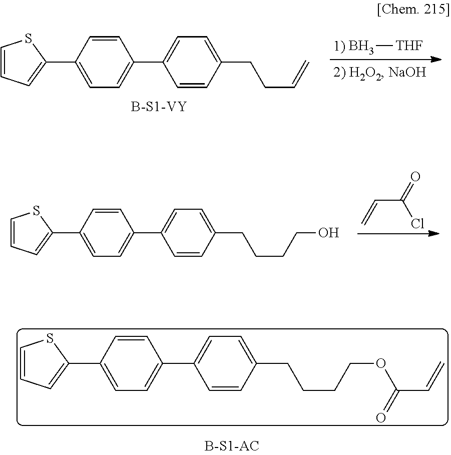

### Example 95

### Synthesis of Ligand: B-S2-AC

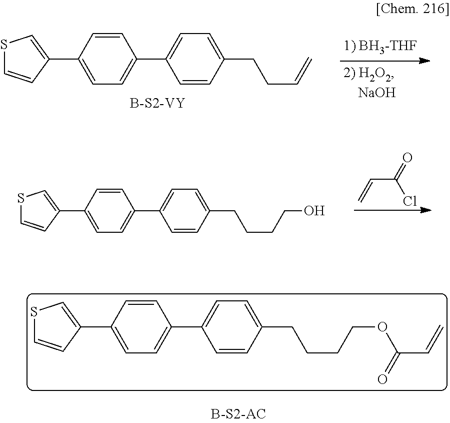

### Example 96

### Synthesis of Ligand: B-P3-AC

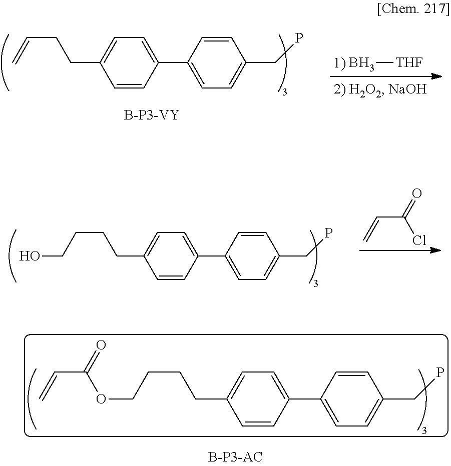

### Example 97

### Synthesis of Ligand: B-N2-AC

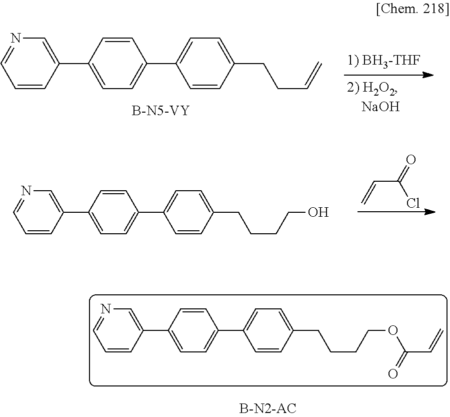

### Example 98

### Synthesis of Ligand: B-N3-AC

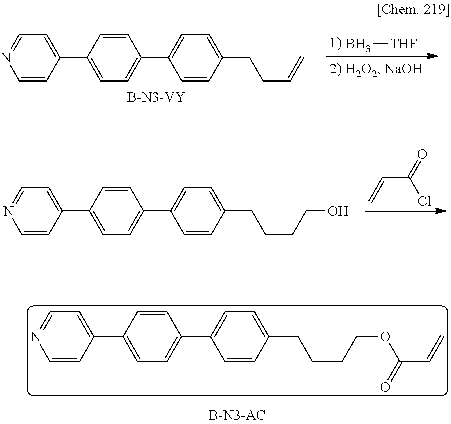

### Example 99

### Synthesis of Ligand: T-S1-C5

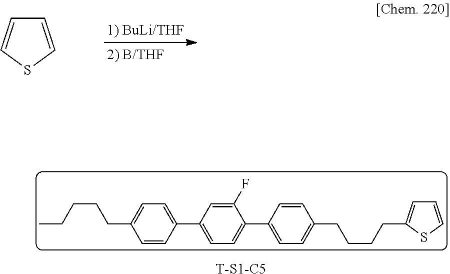

### Example 100

### Synthesis of Ligand: T-S2-C5

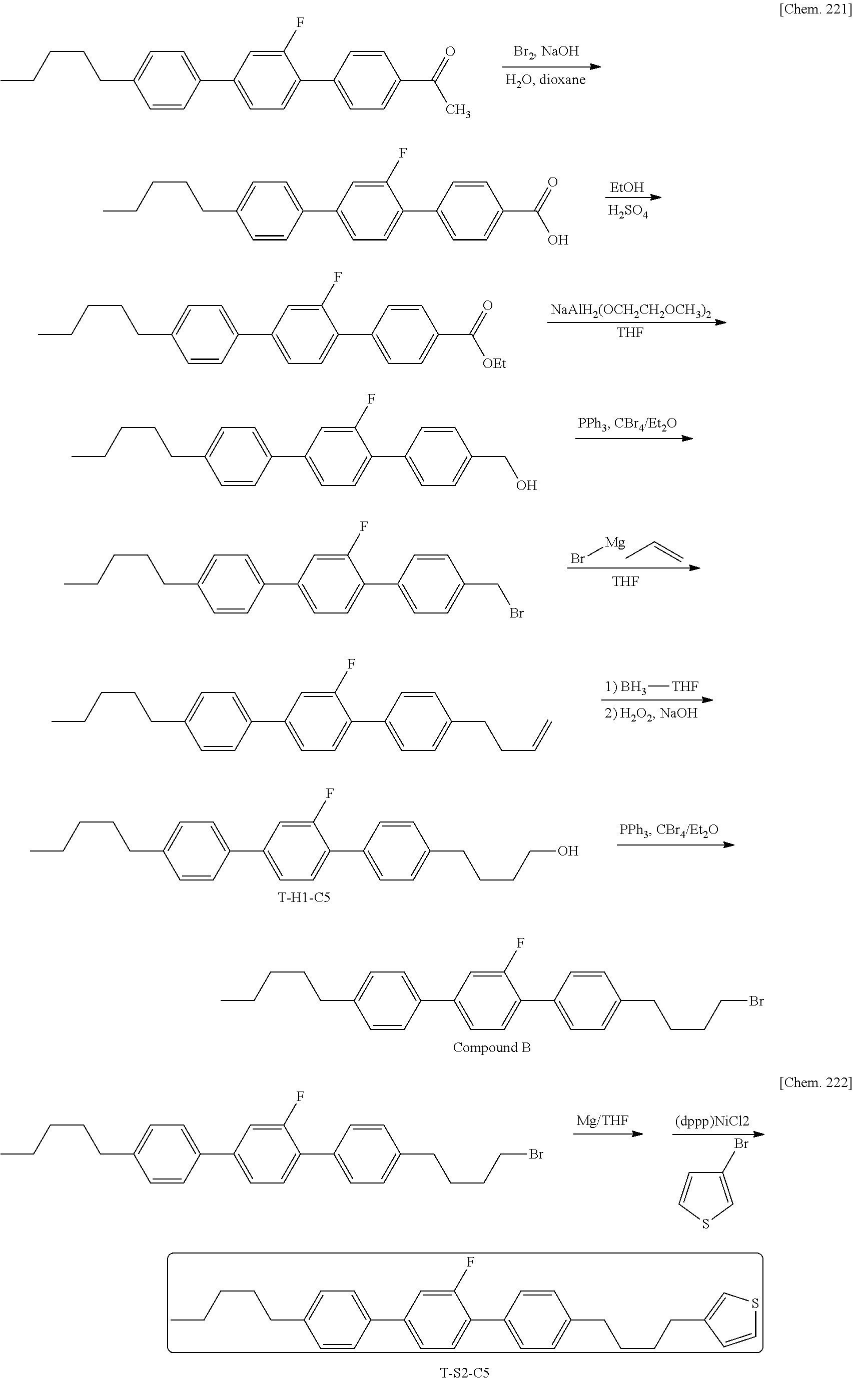

### Example 101

### Synthesis of Ligand: T-S3-C5

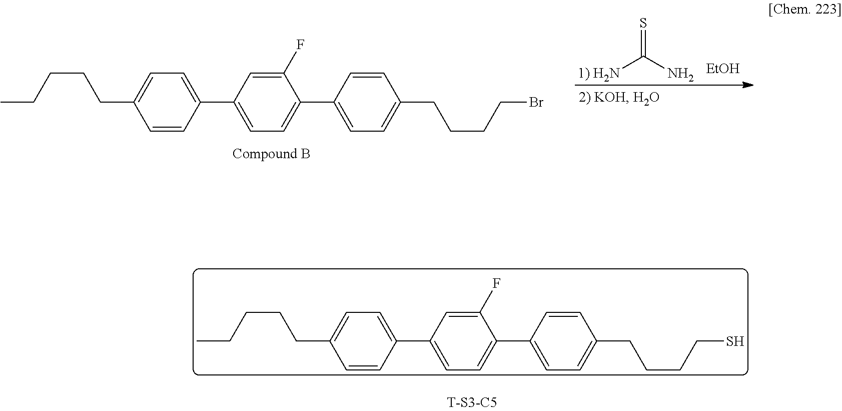

### Example 102

### Synthesis of Ligand: T-P1-C5

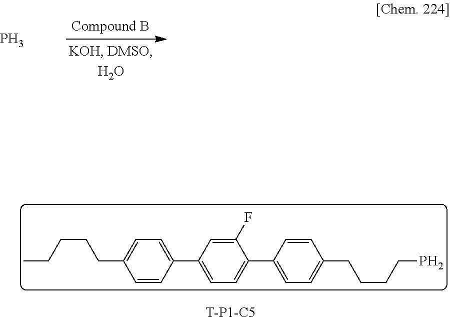

### Example 103

### Synthesis of Ligand: T-P2-C5

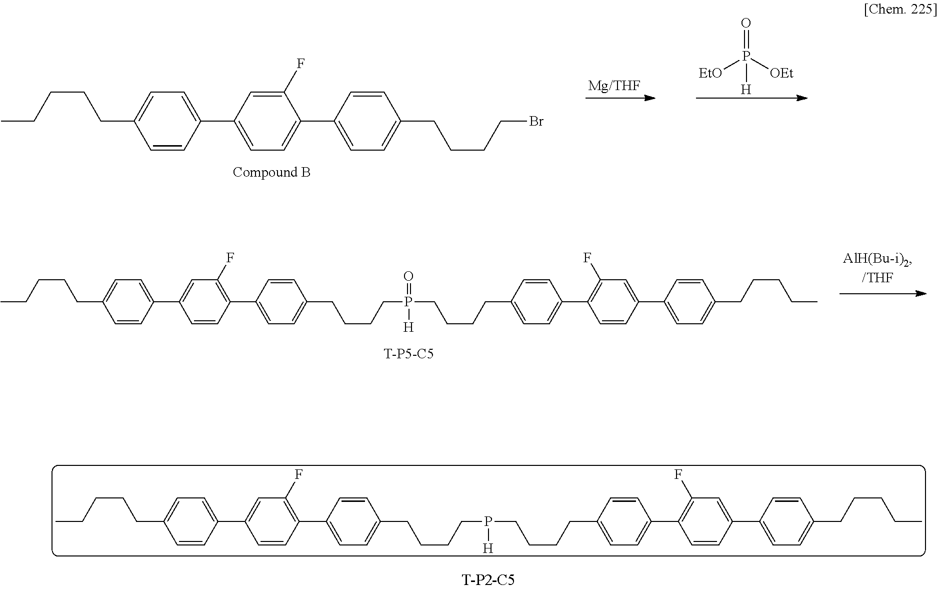

### Example 104

### Synthesis of Ligand: T-P3-C5

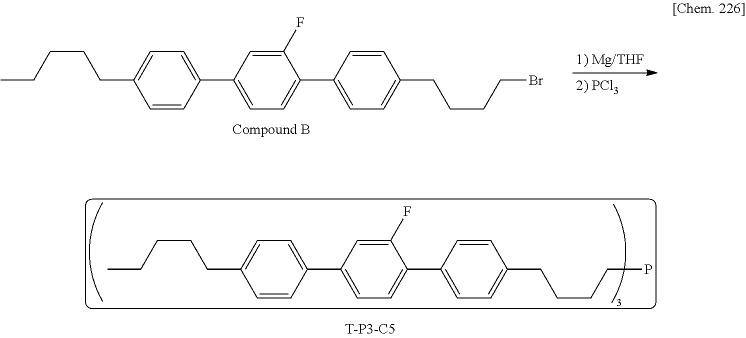

### Example 105

### Synthesis of Ligand: T-P4-C5

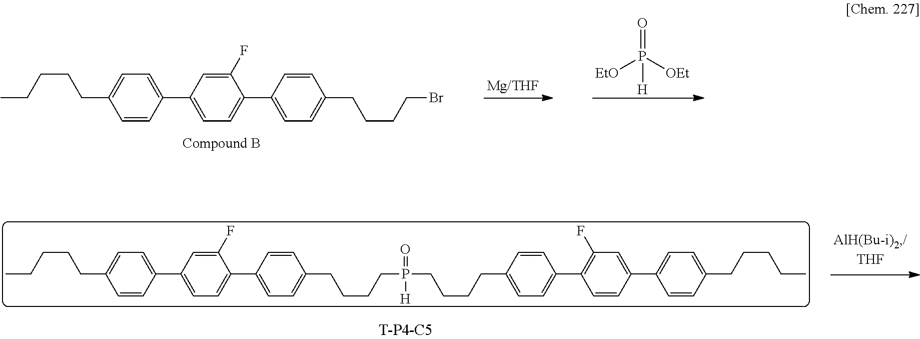 [SMILES: CCCCCc1ccc(-c2ccc(-c3ccc(CCCCBr)cc3)c(F)c2)cc1 ; CCO[PH](=O)OCC ; CCCCCc1ccc(-c2ccc(-c3ccc(CCCC[PH](=O)CCCCc4ccc(-c5ccc(-c6ccc(CCCCC)cc6)cc5F)cc4)cc3)c(F)c2)cc1]

### Example 106

### Synthesis of Ligand: T-P5-C5

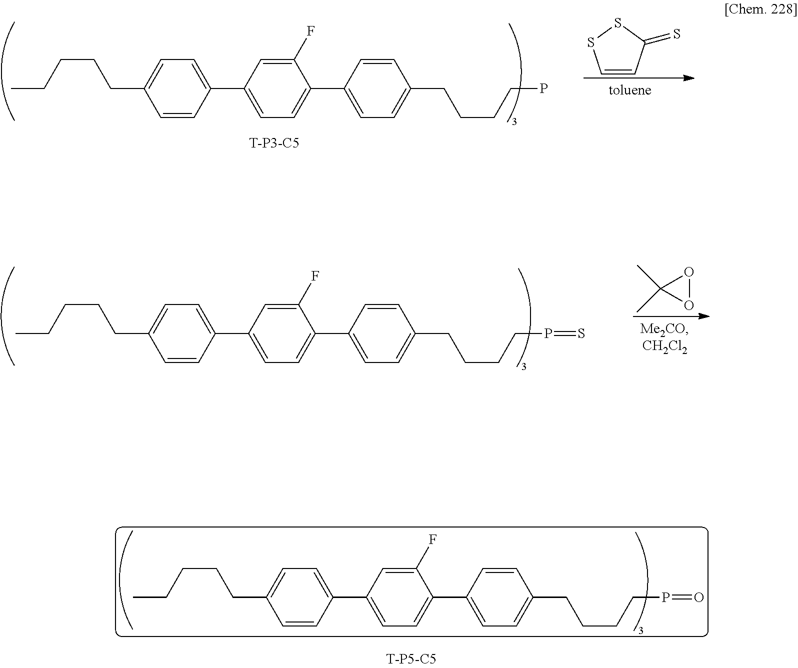

### Example 107

### Synthesis of Ligand: T-N1-C5

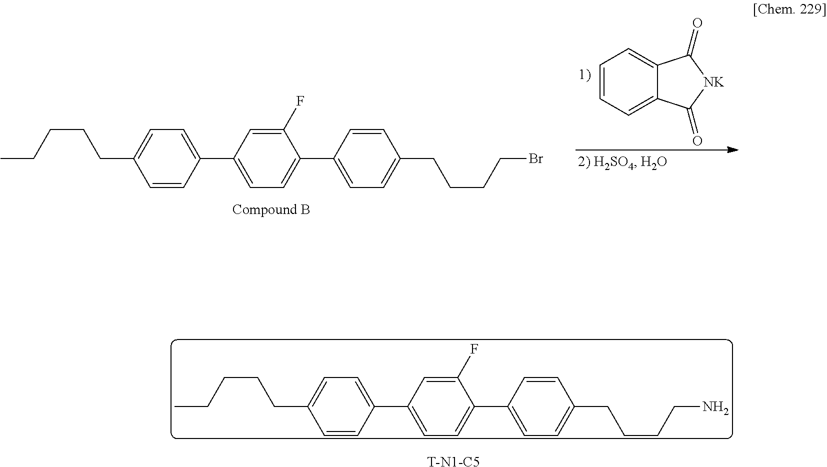

### Example 108

### Synthesis of Ligand: T-N2-C5

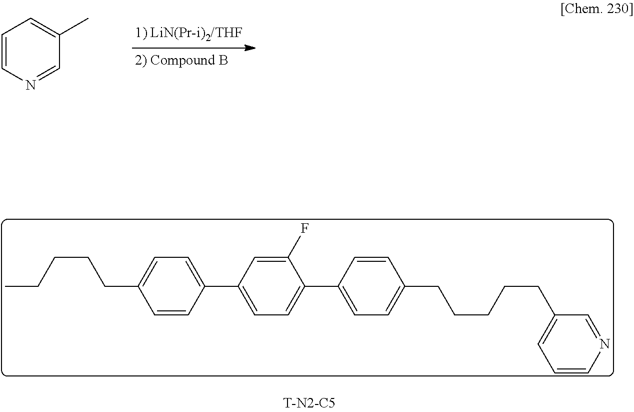

### Example 109

### Synthesis of Ligand: T-H1-C5

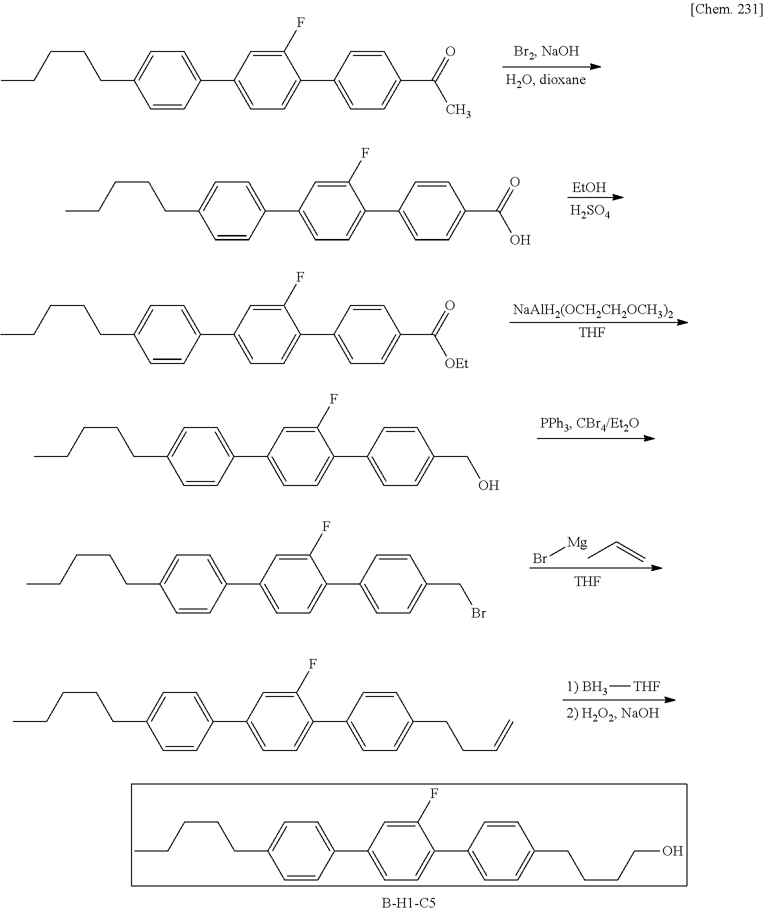

### Example 110

### Synthesis of Ligand: T-H2-C5

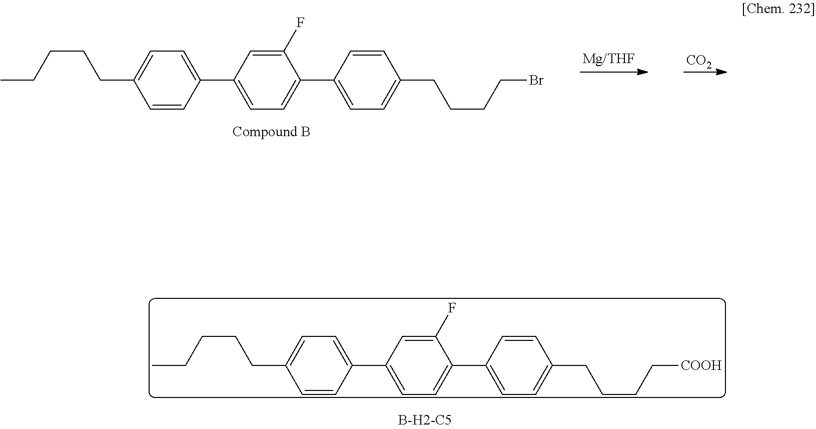 [SMILES: CCCCCc1ccc(-c2ccc(-c3ccc(CCCCBr)cc3)c(F)c2)cc1 ; "CO2" ; CCCCCc1ccc(-c2ccc(-c3ccc(CCCCC(=O)O)cc3)c(F)c2)cc1]

### Example 111

### (Preparation of a Composition Containing a Luminescent Nanocrystal Complex)

Composition 1 for a luminescent nanocrystal complex layer was prepared by mixing the ingredients listed below.

TABLE 1

| | |
|---|---|
| Epoxy acrylate (product name “UNIDIC | 99 parts by mass |
| V-5500,” DIC) | |
| InP/ZnS luminescent nanocrystal complex in Example | 0.4 parts by mass |
| 1 (surface-modifying compound, B-S3-C5; green) | |
| Photopolymerization initiator (product name | 1 part by mass |
| “OXE-02,” BASF Japan) | |

### (Production of a Wavelength Conversion Film)

First, the side of a first barrier film on which it had a silica overlayer was coated with composition 1 for a luminescent nanocrystal complex, prepared as above, using a bar coater, and then the first and a second barrier film were stuck together. The barrier films were commercially available silica-coated films (trade name “TECHBARRIER LX,” Mitsubishi Plastics). The coating was cured by irradiating it with ultraviolet radiation using a conveyor UV system (GS Yuasa International Ltd.) (conveyor speed, 6 m/min; 80 W/cm²) to give a wavelength conversion film composed of two barrier films and a luminescent nanocrystal complex layer sandwiched therebetween.

### (Preparation of a Dispersibility Test Sample and Dispersibility Test)

A sample of the luminescent nanocrystal complex for dispersibility testing was prepared in the same way as wavelength conversion film 1 except that only one barrier film was used instead of two barrier films stuck together. The sample was tested for dispersibility using a transmission electron microscope (TEM).

### (Brightness Tests)

### 1. Test for Initial Brightness

The wavelength conversion film was put on the backlight of a commercially available Kindle Fire HDX7 (blue light-emitting diodes with a peak emission wavelength of 450 nm), and the brightness (K0) of the transmitted light was measured perpendicular to the film surface (“SR-LEDW,” TOPCON).

### 2. Test for Brightness Variability

The brightness after a 250-hour continuous irradiation with the blue LDEs (K1) was measured to check for any change in brightness. The change in brightness was calculated using the equation below.

ΔK[%]=(K0−K1)/K0×100

3. The tests for initial brightness and brightness variability were graded on the basis of the proportion of the change in brightness in an Example to that in a Comparative Example.

Initial brightness test=ΔK(Example)/ΔK(Comparative Example)×100

Brightness change test=ΔK(Example)/ΔK(Comparative Example)×100

The grading criteria were as follows.

⊙ 120% or more

◯ 101% or more and less than 120%

x 100% or less

### Examples 112 to 183

Compositions containing a luminescent nanocrystal complex were prepared as in Example 111 except that the InP/ZnS luminescent nanocrystal complex in Example 1 (ligand, B-S3-C5; green) was replaced with an InP/ZnS luminescent nanocrystal complex in Examples 2 to 70, the CdSe luminescent nanocrystal complex in Example 71 or 72, or the CdSe nanorod phosphor in Example 73. Then the preparation of a wavelength conversion film and a sample for dispersibility testing, a dispersibility test, and brightness tests were carried out as in Example 111.

In addition to this, in Comparative Examples 1 to 5, compositions containing a luminescent nanocrystal complex were prepared in accordance with the formulae given in the tables below, and wavelength conversion films were produced therewith. Then the preparation of a sample for dispersibility testing, a dispersibility test, and brightness tests were carried out in the same way as in Example 111.

TABLE 2

| | Example | Example | Example | Example |
|---|---|---|---|---|
| | 111 | 113 | 115 | 117 |
| Luminescent nanocrystal | Example 1 | Example 3 | Example 5 | Example 7 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-S3-C5 | B-S1-C5 | B-S2-C5 | B-P1-C5 |
| compound | | | | |
| Shape | Granular | Granular | Granular | Granular |
| Color of emitted light | Green | Green | Green | Green |
| Dispersibility | ◯ | ◯ | ◯ | ◯ |
| Initial brightness | ◯ | ◯ | ◯ | ◯ |
| Brightness variability | ◯ | ◯ | ◯ | ◯ |
| | Example | Example | Example | Example |
| | 112 | 114 | 116 | 118 |
| Luminescent nanocrystal | Example 2 | Example 4 | Example 6 | Example 8 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-S3-C5 | B-S1-C5 | B-S2-C5 | B-P1-C5 |
| compound | | | | |
| Shape | Granular | Granular | Granular | Granular |
| Color of emitted light | Red | Red | Red | Red |
| Dispersibility | ◯ | ◯ | ◯ | ◯ |
| Initial brightness | ◯ | ◯ | ◯ | ◯ |
| Brightness variability | ◯ | ◯ | ◯ | ◯ |

TABLE 3

| | Example 119 | Example 121 | Example 123 | Example 125 |
|---|---|---|---|---|
| Luminescent nanocrystal | Example 9 | Example 11 | Example 13 | Example 15 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-P2-C5 | B-P3-C5 | B-P4-C5 | B-P5-C5 |
| compound | | | | |
| Shape | Granular | Granular | Granular | Granular |
| Color of emitted light | Green | Green | Green | Green |
| Dispersibility | ◯ | ◯ | ◯ | ◯ |
| Initial brightness | ◯ | ◯ | ◯ | ◯ |
| Brightness variability | ◯ | ◯ | ◯ | ◯ |
| | Example 120 | Example 122 | Example 124 | Example 126 |
| Luminescent nanocrystal | Example 10 | Example 12 | Example 14 | Example 16 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-P2-C5 | B-P3-C5 | B-P4-C5 | B-P5-C5 |
| compound | | | | |
| Shape | Granular | Granular | Granular | Granular |
| Color of emitted light | Red | Red | Red | Red |
| Dispersibility | ◯ | ◯ | ◯ | ◯ |
| Initial brightness | ◯ | ◯ | ◯ | ◯ |
| Brightness variability | ◯ | ◯ | ◯ | ◯ |

TABLE 4

| | Example 127 | Example 129 | Example 131 | Example 133 |
|---|---|---|---|---|
| Luminescent nanocrystal | Example 17 | Example 19 | Example 21 | Example 23 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-S1-VY | B-S2-VY | B-S3-VY | B-P3-VY |
| compound | | | | |
| Shape | Granular | Granular | Granular | Granular |
| Color of emitted light | Green | Green | Green | Green |
| Dispersibility | ◯ | ◯ | ◯ | ◯ |
| Initial brightness | ◯ | ◯ | ◯ | ◯ |
| Brightness variability | ⊙ | ⊙ | ⊙ | ⊙ |
| | Example 128 | Example 130 | Example 132 | Example 134 |
| Luminescent nanocrystal | Example 18 | Example 20 | Example 22 | Example 24 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | B-S1-VY | B-S2-VY | B-S3-VY | B-P3-VY |
| compound | | | | |
| Shape | Granular | Granular | Granular | Granular |
| Color of emitted light | Red | Red | Red | Red |
| Dispersibility | ◯ | ◯ | ◯ | ◯ |
| Initial brightness | ◯ | ◯ | ◯ | ◯ |
| Brightness variability | ⊙ | ⊙ | ⊙ | ⊙ |

TABLE 5

| | Example 135 | Example 137 | Example 139 |
|---|---|---|---|
| Luminescent nanocrystal | Example 25 | Example 27 | Example 29 |
| complex | | | |
| Method for synthesis | Ligand exchange | Ligand exchange | Ligand exchange |
| Material(s) for the | InP/ZnS | InP/ZnS | InP/ZnS |
| luminescent nanocrystals | | | |
| Surface-modifying | B-S1-AC | B-S2-AC | B-P3-AC |
| compound | | | |
| Shape | Granular | Granular | Granular |
| Color of emitted light | Green | Green | Green |
| Dispersibility | ◯ | ◯ | ◯ |
| Initial brightness | ◯ | ◯ | ◯ |
| Brightness variability | ⊙ | ⊙ | ⊙ |
| | Example 136 | Example 138 | Example 140 |
| Luminescent nanocrystal | Example 26 | Example 28 | Example 30 |
| complex | | | |
| Method for synthesis | Ligand exchange | Ligand exchange | Ligand exchange |
| Material(s) for the | InP/ZnS | InP/ZnS | InP/ZnS |
| luminescent nanocrystals | | | |
| Surface-modifying | B-S1-AC | B-S2-AC | B-P3-AC |
| compound | | | |
| Shape | Granular | Granular | Granular |
| Color of emitted light | Red | Red | Red |
| Dispersibility | ◯ | ◯ | ◯ |
| Initial brightness | ◯ | ◯ | ◯ |
| Brightness variability | ⊙ | ⊙ | ⊙ |

TABLE 6

| | Example 141 | Example 143 | Example 145 | Example 147 |
|---|---|---|---|---|
| Luminescent nanocrystal | Example 31 | Example 33 | Example 35 | Example 37 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | T-S1-C5 | T-S2-C5 | T-S3-C5 | T-P1-C5 |
| compound | | | | |
| Shape | Granular | Granular | Granular | Granular |
| Color of emitted light | Green | Green | Green | Green |
| Dispersibility | ◯ | ◯ | ◯ | ◯ |
| Initial brightness | ◯ | ◯ | ◯ | ◯ |
| Brightness variability | ◯ | ◯ | ◯ | ◯ |
| | Example 142 | Example 144 | Example 146 | Example 148 |
| Luminescent nanocrystal | Example 32 | Example 34 | Example 36 | Example 38 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | T-S1-C5 | T-S2-C5 | T-S3-C5 | T-P1-C5 |
| compound | | | | |
| Shape | Granular | Granular | Granular | Granular |
| Emitted light | Red | Red | Red | Red |
| Dispersibility | ◯ | ◯ | ◯ | ◯ |
| Initial brightness | ◯ | ◯ | ◯ | ◯ |
| Brightness variability | ◯ | ◯ | ◯ | ◯ |

TABLE 7

| | Example 149 | Example 151 | Example 153 | Example 155 |
|---|---|---|---|---|
| Luminescent nanocrystal | Example 39 | Example 41 | Example 43 | Example 45 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | T-P2-C5 | T-P3-C5 | T-P4-C5 | T-P5-C5 |
| compound | | | | |
| Shape | Granular | Granular | Granular | Granular |
| Color of emitted light | Green | Green | Green | Green |
| Dispersibility | ◯ | ◯ | ◯ | ◯ |
| Initial brightness | ◯ | ◯ | ◯ | ◯ |
| Brightness variability | ◯ | ◯ | ◯ | ◯ |
| | Example 150 | Example 152 | Example 154 | Example 156 |
| Luminescent nanocrystal | Example 40 | Example 42 | Example 44 | Example 46 |
| complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent nanocrystals | | | | |
| Surface-modifying | T-P2-C5 | T-P3-C5 | T-P4-C5 | T-P5-C5 |
| compound | | | | |
| Shape | Granular | Granular | Granular | Granular |
| Emitted light | Red | Red | Red | Red |
| Dispersibility | ◯ | ◯ | ◯ | ◯ |
| Initial brightness | ◯ | ◯ | ◯ | ◯ |
| Brightness variability | ◯ | ◯ | ◯ | ◯ |

TABLE 8

| | Example 157 | Example 159 | Example 161 | Example 163 |
|---|---|---|---|---|
| Luminescent nanocrystal | Example 47 | Example 49 | Example 51 | Example 53 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | B-H2-C5 | B-N1-C5 | B-N2-C5 | B-H1-C5 |
| compound | | | | |
| Shape | Granular | Granular | Granular | Granular |
| Emitted light | Green | Green | Green | Green |
| Dispersibility | ◯ | ◯ | ◯ | ◯ |
| Initial brightness | ⊙ | ⊙ | ⊙ | ⊙ |
| Brightness variability | ◯ | ◯ | ◯ | ◯ |
| | Example 158 | Example 160 | Example 162 | Example 164 |
| Luminescent nanocrystal | Example 48 | Example 50 | Example 52 | Example 54 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | B-H2-C5 | B-N1-C5 | B-N2-C5 | B-H1-C5 |
| compound | | | | |
| Shape | Granular | Granular | Granular | Granular |
| Emitted light | Red | Red | Red | Red |
| Dispersibility | ◯ | ◯ | ◯ | ◯ |
| Initial brightness | ⊙ | ⊙ | ⊙ | ⊙ |
| Brightness variability | ◯ | ◯ | ◯ | ◯ |

TABLE 9

| | Example 165 | Example 167 | Example 169 | Example 171 |
|---|---|---|---|---|
| Luminescent nanocrystal | Example 55 | Example 57 | Example 59 | Example 61 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | B-N2-VY | B-N3-VY | B-H1-VY | B-H2-VY |
| compound | | | | |
| Shape | Granular | Granular | Granular | Granular |
| Emitted light | Green | Green | Green | Green |
| Dispersibility | ◯ | ◯ | ◯ | ◯ |
| Initial brightness | ⊙ | ⊙ | ⊙ | ⊙ |
| Brightness variability | ⊙ | ⊙ | ⊙ | ⊙ |
| | Example 166 | Example 168 | Example 170 | Example 172 |
| Luminescent nanocrystal | Example 56 | Example 58 | Example 60 | Example 62 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | B-N2-VY | B-N3-VY | B-H1-VY | B-H2-VY |
| compound | | | | |
| Shape | Granular | Granular | Granular | Granular |
| Emitted light | Red | Red | Red | Red |
| Dispersibility | ◯ | ◯ | ◯ | ◯ |
| Initial brightness | ⊙ | ⊙ | ⊙ | ⊙ |
| Brightness variability | ⊙ | ⊙ | ⊙ | ⊙ |

TABLE 10

| | Example 173 | Example 175 | Example 177 | Example 179 |
|---|---|---|---|---|
| Luminescent nanocrystal | Example 63 | Example 65 | Example 67 | Example 69 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | T-N1-C5 | T-N2-C5 | T-H1-C5 | T-H2-C5 |
| compound | | | | |
| Shape | Granular | Granular | Granular | Granular |
| Emitted light | Green | Green | Green | Green |
| Dispersibility | ◯ | ◯ | ◯ | ◯ |
| Initial brightness | ⊙ | ⊙ | ⊙ | ⊙ |
| Brightness variability | ◯ | ◯ | ◯ | ◯ |
| | Example 174 | Example 176 | Example 178 | Example 180 |
| Luminescent nanocrystal | Example 64 | Example 66 | Example 68 | Example 70 |
| complex | | | | |
| Method for synthesis | Capping | Capping | Capping | Capping |
| Material(s) for the | InP/ZnS | InP/ZnS | InP/ZnS | InP/ZnS |
| luminescent nanocrystals | | | | |
| Surface-modifying | T-N1-C5 | T-N2-C5 | T-H1-C5 | T-H2-C5 |
| compound | | | | |
| Shape | Granular | Granular | Granular | Granular |
| Emitted light | Red | Red | Red | Red |
| Dispersibility | ◯ | ◯ | ◯ | ◯ |
| Initial brightness | ⊙ | ⊙ | ⊙ | ⊙ |
| Brightness variability | ◯ | ◯ | ◯ | ◯ |

TABLE 11

| | Example 181 | Example 182 |
|---|---|---|
| Luminescent nanocrystal complex | Example 71 | Example 72 |
| Method for synthesis | Ligand exchange | Ligand exchange |
| Material(s) for the luminescent | CdSe | CdSe |
| nanocrystals | | |
| Surface-modifying compound | B-S1-C5 | B-S1-C5 |
| Shape | Granular | Granular |
| Emitted light | Green | Red |
| Dispersibility | ◯ | ◯ |
| Initial brightness | ◯ | ◯ |
| Brightness variability | ◯ | ◯ |

| | Example 183 |
|---|---|
| Luminescent nanocrystal complex | Example 73 |
| Method for synthesis | Ligand exchange |
| Material(s) for the luminescent | CdSe |
| nanocrystals | |
| Surface-modifying compound | B-S1-C5 |
| Shape | Rod-shaped |
| Emitted light | Red |
| Dispersibility | ◯ |
| Initial brightness | ◯ |
| Brightness variability | ◯ |

TABLE 12

| | Comparative | Comparative | Comparative | Comparative |
|---|---|---|---|---|
| | Example 1 | Example 2 | Example 3 | Example 4 |
| Luminescent nanocrystals + | Commercial | Commercial | Commercial | Commercial |
| Ligand | product | product | product | product |
| Method for synthesis | — | — | — | — |
| Luminescent nanocrystals | InP/ZnS | InP/ZnS | CdSe | CdSe |
| Surface-modifying compound | Oleylamine | Oleylamine | Octadecylamine | Octadecylamine |
| Shape | Granular | Granular | Granular | Granular |
| Emitted light | Green | Red | Green | Red |
| Dispersibility | X | X | X | X |
| Initial brightness | X | X | X | X |
| Brightness variability | X | X | X | X |

TABLE 13

| | Comparative Example 5 |
|---|---|
| Luminescent nanocrystals + Ligand | Commercial product |
| Method for synthesis | — |
| Luminescent nanocrystals | CdSe |
| Surface-modifying compound | TOPO |
| Shape | Rod-shaped |
| Emitted light | Red |
| Dispersibility | X |
| Initial brightness | X |
| Brightness variability | X |

### About Examples 111 to 156

In Examples 111 to 126, in which the surface of core-shell luminescent nanocrystals was modified with a structurally mesogenic ligand having a biphenyl backbone by ligand exchange, an improvement was observed in all of the characteristics of dispersibility, initial brightness, and brightness variability in comparison with those in Comparative Example 1 or 2, in which the surface modification was with a ligand (surface-modifying compound) that was not structurally mesogenic.

As shown by Examples 127 to 140, modifying the surface of core-shell luminescent nanocrystals with a structurally mesogenic surface-modifying compound (also referred to as a ligand) having a biphenyl backbone by ligand exchange improved all of the characteristics of dispersibility, initial brightness, and brightness variability in comparison with those in Comparative Example 1 or 2 if the surface-modifying compound had polymerizable group(s). In particular, brightness variability was greatly improved in comparison with that with a ligand having no polymerizable group. This appears to be an effect of the formation of a polymer between the surface-modifying compound on the luminescent nanocrystals, modifying the surface of the luminescent nanocrystal, and, for example, a neighboring surface-modifying compound or polymerizable compound induced by ultraviolet irradiation of the polymerizable group(s) of the surface-modifying compound.

In Examples 141 to 156, in which the surface of core-shell luminescent nanocrystals was modified with a structurally mesogenic surface-modifying compound having a fluorinated terphenyl backbone by ligand exchange, an improvement was observed in all of the characteristics of dispersibility, initial brightness, and brightness variability in comparison with those in Comparative Example 1 or 2.

### About Examples 157 to 180

In Examples 157 to 164, in which the surface of a core-shell nanoparticle phosphor was capped with a surface-modifying compound during synthesis (capping) was modified with a structurally mesogenic surface-modifying compound having a biphenyl backbone, an improvement was observed in all of the characteristics of dispersibility, initial brightness, and brightness variability in comparison with those in Comparative Example 1 or 2.

As shown by Examples 165 to 172, capping core-shell luminescent nanocrystals during their synthesis improved all of the characteristics of dispersibility, initial brightness, and brightness variability in comparison with those in Comparative Example 1 or 2 if the surface-modifying compound had a polymerizable group. In particular, brightness variability was greatly improved in comparison with that with a ligand having no polymerizable group. This appears to be an effect of the covering of the surface of the luminescent nanocrystals with a polymer of the surface-modifying compound, modifying the surface of the luminescent nanocrystals, as a result of the polymerization of the polymerizable group of the surface-modifying compound induced by ultraviolet irradiation.

In Examples 173 to 180, in which the surface of core-shell luminescent nanocrystals was capped with a surface-modifying compound during synthesis was modified with a structurally mesogenic surface-modifying compound having a fluorinated terphenyl backbone, an improvement was observed in all of the characteristics of dispersibility, initial brightness, and brightness variability in comparison with those in Comparative Example 1 or 2.

### About Examples 180 to 182

In Example 181 or 182, in which the surface of core-type luminescent nanocrystals was modified with a structurally mesogenic surface-modifying compound having a biphenyl backbone by ligand exchange, an improvement was observed in all of the characteristics of dispersibility, initial brightness, and brightness variability in comparison with those in Comparative Example 3 or 4, in which the surface modification was with a surface-modifying compound that was not structurally mesogenic.

### Example 183

As shown by Example 183, even in the case in which the luminescent nanocrystals were shaped like rods, an improvement was observed in all of the characteristics of dispersibility, initial brightness, and brightness variability in comparison with Comparative Example 5, as in the cases of granular nanocrystals.

### Examples 184 to 210

Compositions containing a luminescent nanocrystal complex were prepared as in Example 111 except that epoxy acrylate, used in Example 111, was replaced with polymerizable liquid crystal compositions 1 to 3 specified below. The preparation of a wavelength conversion film and a sample for dispersibility testing, a dispersibility test, and brightness tests were carried out as in Example 111.

### (Preparation of Polymerizable Liquid Crystal Compositions)

Polymerizable compositions 1 to 3, whose formulae are given in the table below, were prepared by mixing ingredients listed below.

TABLE 14

| | | Polym- | Polym- | Polym- |
|---|---|---|---|---|
| | | erizable | erizable | erizable |
| | | liquid | liquid | liquid |
| | | crystal | crystal | crystal |
| | | compo- | compo- | compo- |
| | | sition 1 | sition 2 | sition 3 |
| UCL1 | 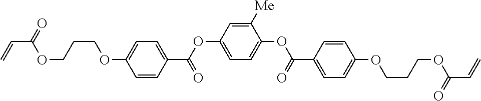 [SMILES: C=CC(=O)OCCCOc1ccc(C(=O)Oc2ccc(OC(=O)c3ccc(OCCCOC(=O)C=C)cc3)c(C)c2)cc1] | 25 | | 20 |
| UCL2 | 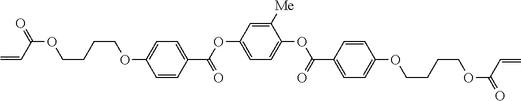 [SMILES: C=CC(=O)OCCCCOc1ccc(C(=O)Oc2ccc(OC(=O)c3ccc(OCCCCOC(=O)C=C)cc3)c(C)c2)cc1] | 25 | | |
| UCL3 | 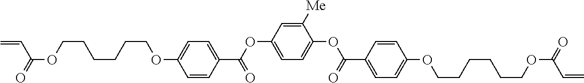 [SMILES: C=CC(=O)OCCCCCCOc1ccc(C(=O)Oc2ccc(OC(=O)c3ccc(OCCCCCCOC(=O)C=C)cc3)c(C)c2)cc1] | | | 20 |
| UCL4 | 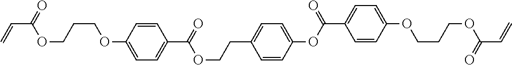 [SMILES: C=CC(=O)OCCCOc1ccc(C(=O)OCCc2ccc(OC(=O)c3ccc(OCCCOC(=O)C=C)cc3)cc2)cc1] | | 25 | |
| UCL5 | 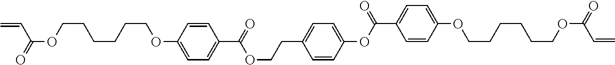 [SMILES: C=CC(=O)OCCCCCCOc1ccc(C(=O)OCCc2ccc(OC(=O)c3ccc(OCCCCCCOC(=O)C=C)cc3)cc2)cc1] | | 25 | |
| UCL6 | 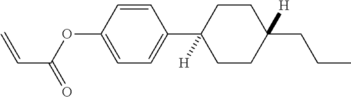 [SMILES: C=CC(=O)Oc1ccc([C@H]2CC[C@H](CCC)CC2)cc1] | 25 | 20 | |
| UCL7 | 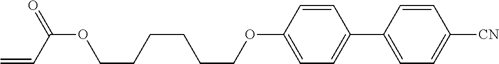 [SMILES: C=CC(=O)OCCCCCCOc1ccc(-c2ccc(C#N)cc2)cc1] | | 10 | |
| UCL8 | 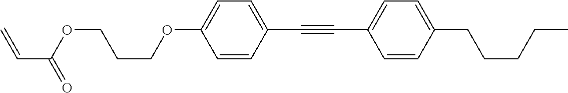 [SMILES: C=CC(=O)OCCCOc1ccc(C#Cc2ccc(CCCCC)cc2)cc1] | 25 | 20 | |
| UCL9 | 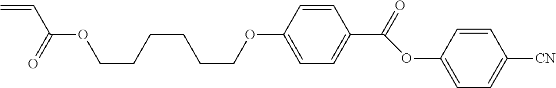 [SMILES: C=CC(=O)OCCCCCCOc1ccc(C(=O)Oc2ccc(C#N)cc2)cc1] | | | 30 |
| UCL10 | 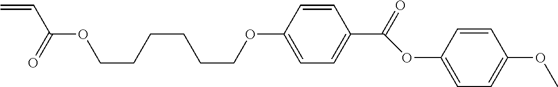 [SMILES: C=CC(=O)OCCCCCCOc1ccc(C(=O)Oc2ccc(OC)cc2)cc1] | | | 30 |

TABLE 15

| | Example 184 | Example 186 | Example 188 | Example 190 |
|---|---|---|---|---|
| Binder | Polymerizable | Polymerizable | Polymerizable | Polymerizable |
| | liquid crystal | liquid crystal | liquid crystal | liquid crystal |
| | composition 1 | composition 2 | composition 3 | composition 1 |
| Luminescent | Example 1 | Example 1 | Example 1 | Example 25 |
| nanocrystal complex | | | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent | | | | |
| nanocrystals | | | | |
| Ligand | B-S3-C5 | B-S3-C5 | B-S3-C5 | B-S1-AC |
| Shape | Granular | Granular | Granular | Granular |
| Color of emitted light | Green | Green | Green | Green |
| Dispersibility | ⊙ | ⊙ | ⊙ | ⊙ |
| Initial brightness | ◯ | ◯ | ◯ | ◯ |
| Brightness variability | ◯ | ◯ | ◯ | ⊙ |
| | Example 185 | Example 187 | Example 189 | Example 191 |
| Composition | Polymerizable | Polymerizable | Polymerizable | Polymerizable |
| | liquid crystal | liquid crystal | liquid crystal | liquid crystal |
| | composition 1 | composition 2 | composition 3 | composition 1 |
| Luminescent | Nanoparticles | Nanoparticles | Example 2 | Example 26 |
| nanocrystal complex | in Example 2 | in Example 2 | | |
| Method for synthesis | Ligand | Ligand | Ligand | Ligand |
| | exchange | exchange | exchange | exchange |
| Material(s) for the | InP/ZnS | InP/ZnS | InP/ZnS | InP/ZnS |
| luminescent | | | | |
| nanocrystals | | | | |
| Ligand | B-S3-C5 | B-S3-C5 | B-S3-C5 | B-S1-AC |
| Shape | Granular | Granular | Granular | Granular |
| Color of emitted light | Red | Red | Red | Red |
| Dispersibility | ⊙ | ⊙ | ⊙ | ⊙ |
| Initial brightness | ◯ | ◯ | ◯ | ◯ |
| Brightness variability | ◯ | ◯ | ◯ | ⊙ |

TABLE 16

| | Example 192 | Example 194 | Example 196 | Example 198 |
|---|---|---|---|---|
| Binder | Polymerizable | Polymerizable | Polymerizable | Polymerizable |
| | liquid crystal | liquid crystal | liquid crystal | liquid crystal |
| | composition 2 | composition 3 | composition 1 | composition 2 |
| Luminescent | Example 25 | Example 25 | Example 47 | Example 47 |
| nanocrystal complex | | | | |
| Method for synthesis | Ligand | Ligand | Capping | Capping |
| | exchange | exchange | | |
| Material(s) for the | InP/ZnS | InP/ZnS | InP/ZnS | InP/ZnS |
| luminescent | | | | |
| nanocrystals | | | | |
| Ligand | B-S1-AC | B-S1-AC | B-H2-C5 | B-H2-C5 |
| Shape | Granular | Granular | Granular | Granular |
| Color of emitted light | Green | Green | Green | Green |
| Dispersibility | ⊙ | ⊙ | ⊙ | ⊙ |
| Initial brightness | ◯ | ◯ | ⊙ | ⊙ |
| Brightness variability | ⊙ | ⊙ | ◯ | ◯ |
| | Example 193 | Example 195 | Example 197 | Example 199 |
| Composition | Polymerizable | Polymerizable | Polymerizable | Polymerizable |
| | liquid crystal | liquid crystal | liquid crystal | liquid crystal |
| | composition 2 | composition 3 | composition 1 | composition 2 |
| Luminescent | Example 26 | Example 26 | Example 48 | Example 48 |
| nanocrystal complex | | | | |
| Method for synthesis | Ligand | Ligand | Capping | Capping |
| | exchange | exchange | | |
| Material(s) for the | InP/ZnS | InP/ZnS | InP/ZnS | InP/ZnS |
| luminescent | | | | |
| nanocrystals | | | | |
| Ligand | B-S1-AC | B-S1-AC | B-H2-C5 | B-H2-C5 |
| Shape | Granular | Granular | Granular | Granular |
| Color of emitted light | Red | Red | Red | Red |
| Dispersibility | ⊙ | ⊙ | ⊙ | ⊙ |
| Initial brightness | ◯ | ◯ | ⊙ | ⊙ |
| Brightness variability | ⊙ | ⊙ | ◯ | ◯ |

TABLE 17

| | Example 200 | Example 202 | Example 204 | Example 206 |
|---|---|---|---|---|
| Binder | Polymerizable | Polymerizable | Polymerizable | Polymerizable |
| | liquid crystal | liquid crystal | liquid crystal | liquid crystal |
| | composition 3 | composition 1 | composition 2 | composition 3 |
| Luminescent | Example 47 | Example 71 | Example 71 | Example 71 |
| nanocrystal complex | | | | |
| Method for synthesis | Capping | Ligand | Ligand | Ligand |
| | | exchange | exchange | exchange |
| Material(s) for the | InP/ZnS | CdSe | CdSe | CdSe |
| luminescent | | | | |
| nanocrystals | | | | |
| Ligand | B-H2-C5 | B-S1-C5 | B-S1-C5 | B-S1-C5 |
| Shape | Granular | Granular | Granular | Granular |
| Color of emitted light | Green | Green | Green | Green |
| Dispersibility | ⊙ | ⊙ | ⊙ | ⊙ |
| Initial brightness | ⊙ | ◯ | ◯ | ◯ |
| Brightness variability | ◯ | ◯ | ◯ | ◯ |
| | Example 201 | Example 203 | Example 205 | Example 207 |
| Composition | Polymerizable | Polymerizable | Polymerizable | Polymerizable |
| | liquid crystal | liquid crystal | liquid crystal | liquid crystal |
| | composition 3 | composition 1 | composition 2 | composition 3 |
| Luminescent | Example 48 | Example 72 | Example 72 | Example 72 |
| nanocrystal complex | | | | |
| Method for synthesis | Capping | Ligand | Ligand | Ligand |
| | | exchange | exchange | exchange |
| Material(s) for the | InP/ZnS | CdSe | CdSe | CdSe |
| luminescent | | | | |
| nanocrystals | | | | |
| Ligand | B-H2-C5 | B-S1-C5 | B-S1-C5 | B-S1-C5 |
| Shape | Granular | Granular | Granular | Granular |
| Color of emitted light | Red | Red | Red | Red |
| Dispersibility | ⊙ | ⊙ | ⊙ | ⊙ |
| Initial brightness | ⊙ | ◯ | ◯ | ◯ |
| Brightness variability | ◯ | ◯ | ◯ | ◯ |

TABLE 18

| | Example 208 | Example 209 | Example 210 |
|---|---|---|---|
| Composition | Polymerizable | Polymerizable | Polymerizable |
| | liquid crystal | liquid crystal | liquid crystal |
| | composition 1 | composition 2 | composition 3 |
| Luminescent | Example 73 | Example 73 | Example 73 |
| nanocrystal complex | | | |
| Method for synthesis | Ligand | Ligand | Ligand |
| | exchange | exchange | exchange |
| Material(s) for the | CdSe | CdSe | CdSe |
| luminescent | | | |
| nanocrystals | | | |
| Ligand | B-S1-C5 | B-S1-C5 | B-S1-C5 |
| Shape | Rod-shaped | Rod-shaped | Rod-shaped |
| Color of emitted light | Red | Red | Red |
| Dispersibility | ⊙ | ⊙ | ⊙ |
| Initial brightness | ◯ | ◯ | ◯ |
| Brightness variability | ◯ | ◯ | ◯ |

### About Examples 184 to 210

The formulae in which polymerizable liquid crystal compositions 1 to 3 were used instead of epoxy acrylate (UNIDIC V-5500) improved dispersibility in particular,

although initial brightness and brightness variability were also improved in comparison with those in Comparative Examples. This appears to owe to increased compatibility of the mesogen of the ligand with that of the binder.

### “Production of Ink Compositions and and Color Filters Using Them Them”

### (Preparation of a Green Light-Emitting Ink Composition)

In a container filled with nitrogen gas, 2.5 g of titanium oxide (MPT141, Ishihara Sangyo Kaisha, Ltd.), 12 g of a mixed solution of a glycidyl-containing solid acrylic resin (a mixed solution of FINEDIC A-254 (DIC Corporation) (6.3 g), 1-methylcyclohexane-4,5-dicarboxylic anhydride (1 g), and dimethylbenzylamine (0.1 g) in 1,4-butanediol diacetate with a nonvolatile content of 30%”)), 1 g of BYK-2164 (BYK), 1,4-butanediol diacetate (Daicel Corporation), and 23 g of a 1,4-butanediol diacetate solution containing InP/ZnS core-shell nanocrystals (green light-emitting) having T-N1-C5, in Example 49, as a ligand (nonvolatile content, 30% by mass) were mixed to give an ink composition.

### (Preparation of a Red Light-Emitting Ink Composition)

A 1,4-butanediol diacetate solution containing InP/ZnS core-shell nanocrystals (red light-emitting) (nonvolatile content, 30% by mass) was produced in the same way as the green light-emitting ink composition except that ligand T-N1-C5, in Example 49, was replaced with ligand T-S3-C5, in Example 36.

### [Production and Testing of Light Conversion Films]

The green light-emitting and red light-emitting ink compositions obtained as above were separately applied to glass substrates (supporting substrates) to a dry thickness of 3 μm using a spin coater in a nitrogen-filled glove box. The applied coatings were cured by heating to 180° C. in nitrogen, forming a red light-emitting light conversion film and a green light-emitting light conversion layer film on the glass substrates as layers of a cured ink composition (light conversion layers).

The ink compositions achieved stable film formation, with no discoloration or fading due to the aggregation of nanocrystals observed for both the green light-emitting and red light-emitting light conversion layer films.

This suggests that a ligand having a mesogenic backbone makes nanocrystals incorporating it less likely to aggregate and reduces concentration quenching by virtue of its large apparent volume, rigid structure, and the resultant small change in excluded volume.

1. A luminescent nanocrystal complex comprising luminescent nanocrystals and a surface-modifying compound modifying a surface of the luminescent nanocrystals, wherein
the surface-modifying compound has a mesogenic group and at least one group that binds to the surface of the luminescent nanocrystals.

2. The luminescent nanocrystal complex according to claim 1, wherein the group of the surface-modifying compound that binds to the surface of the luminescent nanocrystals contains one or two or more types of atoms selected from the group consisting of sulfur, nitrogen, oxygen, and phosphorus.

3. The luminescent nanocrystal complex according to claim 1, wherein the group of the surface-modifying compound that binds to the surface of the luminescent nanocrystals is any one or more of hydroxy, thiol, carboxylic acid, amine, sulfonic acid, phosphine, phosphine oxide, and thioether.

4. The luminescent nanocrystal complex according to claim 1, wherein the surface-modifying compound is general formula (i). 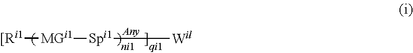
“In general formula (i) above,
MGⁱ¹ represents a mesogenic group,
—S_{P}ⁱ¹ represents a single bond or spacer group,
Rⁱ¹ represents a hydrogen atom, halogen atom, cyano group, C1-18 linear or branched alkyl group, or a group represented by general formula (i-1), where one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—, and one or more hydrogen atoms in the alkyl group may be substituted with general formula (i-1) 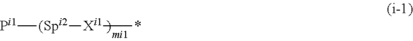
(In general formula (i-1) above, Pⁱ¹ represents a reactive functional group,
Spⁱ² represents a single bond or C1-18 alkylene group, where hydrogen atoms in the alkylene group may be substituted with one or more halogen atoms or CNs, and one CH₂ group present in the alkylene group, or each of nonadjacent two or more independently, may be substituted with —O—, —COO—, —OCO—, or —OCO—O—,
Xⁱ¹ represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P-Spⁱ² and Spⁱ²-X, however, include no —O—O—, —O—NH—, —S—S—, or —O—S— group.), and mi1 represents 0 or 1.),
Wⁱ¹ represents —SH, —PH₂, —PH—, —POH₂, —POH—, —NH₂, —NH—, —OH, —COOH, a group represented by general formula (W-1) to (W-14), or a single bond. 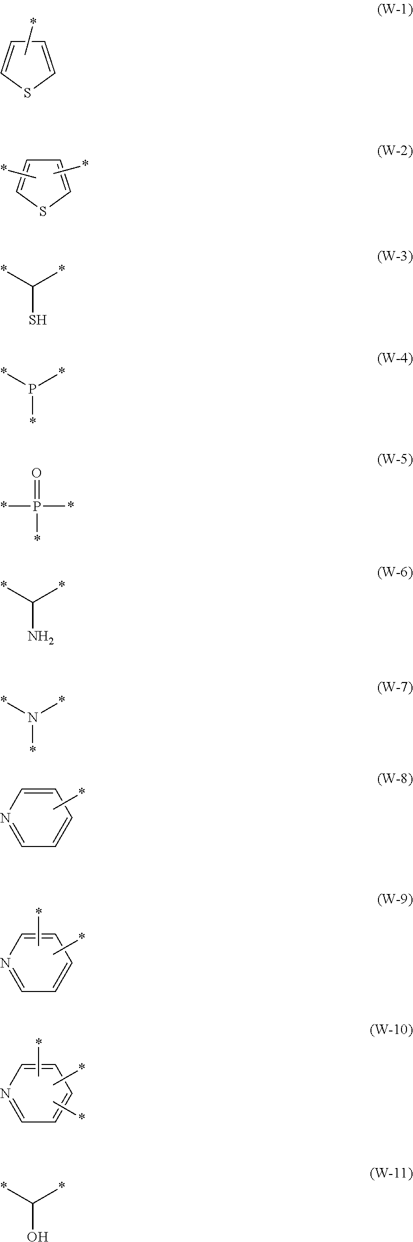 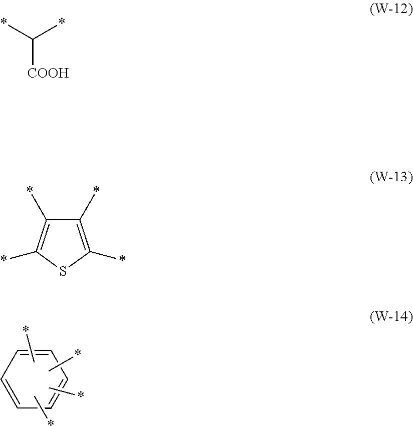
qi1 represents an integer of 1 to 4,
ni1 represents an integer of 0 to 8, and if ni1 is 2 or more and there are multiple MGⁱ¹s or SPⁱ¹s, the MGⁱ¹s or SPⁱ¹s may be the same or different.)”

5. The luminescent nanocrystal complex according to claim 4, wherein MGⁱ¹ in general formula (i) is a divalent organic group incorporating a cyclic group optionally with a hydrogen atom in the cyclic group substituted with general formula (i-3):
Rⁱ²-(MGⁱ²Spⁱ³)ₙᵢ₂- (i-3)
(In general formula (i-3) above, MGⁱ² represents a mesogenic group,
SPⁱ³ represents a single bond or spacer group,
Rⁱ² represents a hydrogen atom, halogen atom, cyano group, or C1-18 linear or branched alkyl group, where one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—, and the hydrogen atom, halogen atom, cyano group, or one or more hydrogen atoms in the alkyl group may be substituted with general formula (i-4)
Pⁱ²-(Spⁱ⁴-Xⁱ²)ₘᵢ₂— (i4)
(In general formula (i-4) above, Pⁱ² represents a reactive functional group,
Spⁱ⁴ represents a single bond or C1-18 alkylene group, where hydrogen atoms in the alkylene group may be substituted with one or more halogen atoms or CNs, and one CH₂ group present in the alkylene group, or each of nonadjacent two or more independently, may be substituted with —O—, —COO—, —OCO—, or —OCO—O—,
Xⁱ² represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P-Spⁱ⁴ and Spⁱ⁴-X, however, include no —O—O—, —O—NH—, —S—S—, or —O—S— group.), and mi2 represents 0 or 1.).

6. The luminescent nanocrystal complex according to claim 4, wherein SPⁱ¹ in general formula (i) is a divalent organic group optionally with a hydrogen atom in the organic group substituted with general formula (i-3):
Rⁱ²-(MGⁱ²-Spⁱ³)ₙᵢ₂- (i-3)
(In general formula (i-3) above, MGⁱ² represents a mesogenic group,
SPⁱ³ represents a single bond or spacer group,
Rⁱ² represents a hydrogen atom, halogen atom, cyano group, or C1-18 linear or branched alkyl group, where one —CH₂— in the alkyl group, or each of nonadjacent two or more independently, may be substituted with —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, —C≡C—, —NH—, —PH—, or —POH—, and the hydrogen atom, halogen atom, cyano group, or one or more hydrogen atoms in the alkyl group may be substituted with general formula (i-4)
Pⁱ²-(Spⁱ⁴-Xⁱ²)ₘᵢ₂— (i-4)
(In general formula (i-4) above, Pⁱ² represents a reactive functional group,
Spⁱ⁴ represents a single bond or C1-18 alkylene group, where hydrogen atoms in the alkylene group may be substituted with one or more halogen atoms or CNs, and one CH₂ group present in the alkylene group, or each of nonadjacent two or more independently, may be substituted with —O—, —COO—, —OCO—, or —OCO—O—,
Xⁱ² represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond (P-Spⁱ⁴ and Spⁱ⁴-X, however, include no —O—O—, —O—NH—, —S—S—, or —O—S— group.), and mi2 represents 0 or 1.).

7. The luminescent nanocrystal complex according to claim 4, wherein in general formula (i), MGⁱ¹ is represented by general formula (i-5).
-(Aⁱ¹-Zⁱ¹)ₙᵢ₃-Aⁱ²- (i-5)
(In general formula (i-5) above, Aⁱ¹ and Aⁱ² each independently represent one ring structure selected from the group consisting of unsubstituted or substituted 1,4-phenylene, 1,4-cyclohexylene, 1,4-cyclohexenyl, tetrahydropyran-2,5-diyl, 1,3-dioxan-2,5-diyl, tetrahydrothiopyran-2,5-diyl, thiophen-2,5-diyl, 1,4-bicyclo(2,2,2)octylene, decahydronaphthalen-2,6-diyl, pyridin-2,5-diyl, pyrimidin-2,5-diyl, pyrazin-2,5-diyl, thiophen-2,5-diyl-, 1,2,3,4-tetrahydronaphthalen-2,6-diyl, 2,6-naphthylene, phenanthren-2,7-diyl, 9,10-dihydrophenanthren-2,7-diyl, 1,2,3,4,4a,9,10a-octahydrophenanthren-2,7-diyl, 1,4-naphthylene, benzo[1,2-b:4,5-b′]dithiophen-2,6-diyl, benzo[1,2-b:4,5-b′]diselenophen-2,6-diyl, [1]benzothieno[3,2-b]thiophen-2,7-diyl, [1]benzoselenopheno[3,2-b]selenophen-2,7-diyl, and fluoren-2,7-diyl groups,
substitution of one or more or two or more hydrogen atoms in the ring structures may be substituted with a substituent selected from the group consisting of fluorine and chlorine atoms, CF₃, OCF₃, CN, nitro, amino, phosphine, phosphonic acid, carboxyl, hydroxy, aldehyde, mercapto, carbamoyl, sulfo, thienyl, pyridyl, C1-8 alkyl, C1-8 alkoxy, C1-8 alkanoyl, C1-8 alkanoyloxy, C2-8 alkenyl, C2-8 alkenyloxy, C1-8 alkenoyl, and C1-8 alkenoyloxy groups, and a substituent represented by general formula (i-1) above, where the C1-8 alkyl, C1-8 alkoxy, C1-8 alkanoyl, C1-8 alkanoyloxy, C2-8 alkenyl, C2-8 alkenyloxy, C1-8 alkenoyl, and C1-8 alkenoyloxy groups and substituent represented by general formula (i-1) may be substituted with a fluorine or chlorine atom or a CF₃, OCF₃, CN, nitro, amino, phosphine, phosphonic acid, carboxyl, hydroxy, aldehyde, mercapto, carbamoyl, sulfo, thienyl, or pyridyl group,
Zⁱ¹ represents —COO—, —OCO—, —CO—S—, —S—CO—, —O—CO—O—, —CH₂CH₂—, —OCH₂—, —CH₂O—, —OCF₂—, —CF₂S—, —SCF₂—, —CH═CH—, —CF═CF—, —C≡C—, —CH═CHCOO—, —OCOCH═CH—, —CH₂CH₂COO—, —CH₂CH₂OCO—, —COOCH₂CH₂—, —OCOCH₂CH₂—, —CONH—, —NHCO—, —N═N—, —CH═N—N═CH—, a halogenated or non-halogenated C2-10 alkyl group, or a single bond,
ni3 represents an integer of 1 to 4, and if ni3 is 2 or more and there are multiple Aⁱ¹s and Zⁱ¹s, the Aⁱ¹s or Zⁱ¹s may be the same or different.)

8. The luminescent nanocrystal complex according to claim 1, wherein the luminescent nanocrystals have a core that contains at least one first semiconductor material; and
a shell that covers the core and contains a second semiconductor material that is the same as or different than in the core.

9. The luminescent nanocrystal complex according to claim 8, wherein the first semiconductor material is one or two or more selected from the group consisting of group II-VI semiconductors, group III-V semiconductors, group I-III-VI semiconductors, group IV semiconductors, and group I-II-IV-VI semiconductors.

10. A composition comprising a luminescent nanocrystal complex according to claim 1 and a binder resin.

11. An optical film comprising a luminescent nanocrystal complex according to claim 1 and a binder resin.

12. A surface-modifying compound comprising the preceding general formula (i) and capability to bind to luminescent nanocrystals.

13. An optical element comprising an optical film according to claim 11.
